SEQUENCE LISTING (16 sequences)

SEQ ID NO: 1 - protein, 19 aa, Homo sapiens
  misc_feature: human FGFR1 fragment
LVLGKPLGEGCFGQVVLAE

SEQ ID NO: 2 - protein, 18 aa, Homo sapiens
  misc_feature: human FGFR2 fragment
LTLGKPLGEGCFGQVVMA

SEQ ID NO: 3 - protein, 18 aa, Homo sapiens
  misc_feature: human FGFR1 fragment
YYKKTTNGRLPVKWMAPE

SEQ ID NO: 4 - protein, 18 aa, Homo sapiens
  misc_feature: human FGFR2 fragment
YYKKTTNGRLPVKWMAPE

SEQ ID NO: 5 - protein, 18 aa, Homo sapiens
  misc_feature: human FGFR1 fragment
EYLQARRPPGLEYCYNPS

SEQ ID NO: 6 - protein, 18 aa, Homo sapiens
  misc_feature: human FGFR2 fragment
EYLRARRPPGMEYSYDIN

SEQ ID NO: 7 - protein, 31 aa, Homo sapiens
  misc_feature: human FGFR1 fragment
PYPGVPVEELFKLLKEGHRMDKPSNCTNELY

SEQ ID NO: 8 - protein, 31 aa, Homo sapiens
  misc_feature: human FGFR2 fragment
PYPGIPVEELFKLLKEGHRMDKPANCTNELY

SEQ ID NO: 9 - protein, 120 aa, Homo sapiens
  misc_feature: human FGFR1 fragment
VSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPP

SEQ ID NO: 10 - protein, 120 aa, Homo sapiens
  misc_feature: human FGFR2 fragment
VSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPP

SEQ ID NO: 11 - protein, 120 aa, Homo sapiens
  misc_feature: human FGFR3 fragment
VSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPP

SEQ ID NO: 12 - protein, 120 aa, Homo sapiens
  misc_feature: human FGFR4 fragment
LVSLDLPLDPLWEFPRDRLVLGKPLGEGCFGQVVRAEAFGMDPARPDQASTVAVKMLKDNASDKDLADLVSEMEVMKLIGRHKNIINLLGVCTQEGPLYVIVECAAKGNLREFLRARRPP

SEQ ID NO: 13 - protein, 120 aa, Homo sapiens
  misc_feature: human FGFR1 fragment
ADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVA

SEQ ID NO: 14 - protein, 120 aa, Homo sapiens
  misc_feature: human FGFR2 fragment
ADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILT

SEQ ID NO: 15 - protein, 120 aa, Homo sapiens
  misc_feature: human FGFR3 fragment
ADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLT

SEQ ID NO: 16 - protein, 119 aa, Homo sapiens
  misc_feature: human FGFR4 fragment
ADFGLARGVHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVL

(19) United States
(12) Patent Application Publication
(10) Pub. No.: US 2012/0328599 A1
(43) Pub. Date: Dec. 27, 2012

(54) INHIBITORS OF RECEPTOR TYROSINE KINASES (RTK) AND METHODS OF USE THEREOF

(75) Inventors: Jae Hyun Bae, Branford, CT (US); Irit Lax, Woodbridge, CT (US); Joseph Schlessinger, Woodbridge, CT (US)

(73) Assignee: YALE UNIVERSITY, New Haven, CT (US)

(21) Appl. No.: 13/519,837

(22) PCT Filed: Jan. 13, 2011
(86) PCT No.: PCT/US11/21109

§ 371 Date: Sep. 11, 2012

Related U.S. Application Data
(60) Provisional application No. 61/335,950, filed on Jan. 14, 2010.

(51) Int. Cl. C07K 16/28; C07D 239/90; C07D 243/24; C07D 281/10; C07D 281/02; C07K 14/00; A61K 31/4375; A61K 31/517; A61K 31/5513; A61K 31/554; A61K 38/16; A61P 19/08; A61P 35/00; A61K 39/395; C12N 5/12; G01N 33/573; C07D 471/04

(52) U.S. Cl. 424/130.1; 546/88; 544/287; 540/510; 540/490; 540/491; 530/300; 530/387.9; 530/387.3; 514/292; 514/266.3; 514/221; 514/211.05; 514/211.06; 514/19.3; 514/16.7; 514/19.5; 514/19.4; 435/331; 435/7.4

(57) ABSTRACT

The present invention provides moieties that bind to the asymmetric contact interface of a receptor tyrosine kinase (RTK), wherein the moieties inhibit ligand induced trans autophosphorylation of the RTK. The present invention also provides methods of treating or preventing an RTK-associated disease and methods for identifying moieties that bind to an asymmetric contact interface of an RTK.

### CROSS-REFERENCE TO RELATED APPLICATIONS

This application is related and claims priority to U.S. Provisional Application Ser. No. 61/335,950, filed Jan. 14, 2010, the entire contents of which are expressly incorporated herein by this reference.

### STATEMENT REGARDING FEDERALLY SPONSORED RESEARCH OR DEVELOPMENT

This invention was made with Government support under contract R01-AR 051448, R01-AR 051886, P50 AR054086, awarded by the National Institutes of Health. The government may have certain rights in the invention.

### BACKGROUND OF THE INVENTION

Ligand induced tyrosine autophosphorylation plays an important role in the control of activation and cell signaling by receptor tyrosine kinases (RTK) (Schlessinger (1988) Trends Biochem. Sci., 3(11):443-447; Schlessinger (2000) Cell, 103(2):211-225; Schlessinger and Lemmon (2003) Sci. STKE, 191:RE12; Schlessinger and Ullrich (1992) Neuron, 9(3):383-391; Lemmon and Schlessinger (1994) Trends Biochem. Sci., 19(11):459-463; and Lemmon and Schlessinger (1998) Methods Mol. Biol., 84:49-71). Structural and biochemical studies have shown that autophosphorylation of receptor tyrosine kinases, such as fibroblast growth factor receptor 1 (FGFR1), are mediated by a sequential and precisely ordered intermolecular reaction that can be divided into three phases (Furdui et al. (2006) Mol. Cell., 21(5):711-717 and Lew et al. (2009) Sci. Signal, 2(58):ra6) and FGFR2 (Chen et al. (2008) Proc. Natl. Acad. Sci. U.S.A., 105(50):19660-19665). For example, the first phase involves trans phosphorylation of a tyrosine located in the activation loop (Y653 in FGFR1) of the catalytic core resulting in 50-100 fold stimulation of kinase activity (Furdui et al., 2006). In the second phase, tyrosine residues that serve as docking sites for signaling proteins are phosphorylated including tyrosines in the kinase insert region (Y583, Y585), the juxtamembrane region (Y463) and in the C-terminal tail (Y766) of FGFR1. In the final and third phase, Y654; a second tyrosine located in the activation loop is phosphorylated, resulting in an additional 10 fold increase in FGFR1 kinase activity (Furdui et al., 2006). Interestingly, tyrosines that are adjacent to one another (e.g., Y653, Y654 and Y583, Y585) are not phosphorylated sequentially, suggesting that both sequence and structural specificities dictate the order of phosphorylation for receptor tyrosine kinases.

Although tyrosine phosphorylation plays a major role in cell signaling, it is not yet clear what the structural basis is for trans autophosphorylation of receptor tyrosine kinases. In other words, the molecular mechanism underlying how one kinase (the enzyme) within the dimerized receptor specifically and sequentially catalyzes phosphorylation of tyrosine(s) of the other kinase (the substrate) has not yet been resolved. Accordingly, there is a need to better characterize the structures, phosphorylation and signaling of RTKs. Such a characterization will lead to the informed identification of regions which may be targeted with drugs, pharmaceuticals, or other biologics.

### SUMMARY OF THE INVENTION

Tyrosine autophosphorylation of receptor tyrosine kinases (RTKs) plays a critical role in the regulation of kinase activity and in the recruitment and activation of intracellular signaling pathways. Autophosphorylation is mediated by a sequential and precisely ordered intermolecular (trans) reaction. The instant invention demonstrates that the formation of an asymmetric dimer between activated RTK kinase domains is required for trans autophosphorylation of the RTK in stimulated cells. In the FGFR1 receptor tyrosine kinase, for example, trans autophosphorylation is mediated by specific asymmetric contacts between the N-lobe of one kinase molecule, which serves as an active enzyme, and specific docking sites on the C-lobe of a second kinase molecule, which serves a substrate. Pathological loss of function mutations or oncogenic activating mutations in the asymmetric contact interface of receptor tyrosine kinases may hinder or facilitate asymmetric dimer formation and trans autophosphorylation, respectively. These data provide the molecular basis underlying the control of trans autophosphorylation of receptor tyrosine kinases, including fibroblast growth factor receptors.

Accordingly, the present invention provides a novel approach for pharmacological inhibition of pathologically activated RTKs, such as FGF receptors, by inhibition of asymmetric tyrosine kinase dimer formation; a step required for RTK autophosphorylation, enzyme activation and cell signaling.

In one aspect, the invention provides a moiety that binds to an asymmetric contact interface of a receptor tyrosine kinase (RTK), wherein the moiety inhibits ligand-induced trans autophosphorylation of the RTK. In one embodiment, the moiety inhibits ligand-induced trans autophosphorylation of the RTK and activation of the RTK. In another embodiment, the moiety does not bind to a nucleotide binding site of a catalytic domain of the RTK. In yet another embodiment, the moiety binds to an asymmetric contact interface on the N-lobe of one monomer of the RTK or to an asymmetric contact interface on the C-lobe of one monomer of the RTK. In one embodiment, the moiety does not cause the loss of intrinsic kinase activity. In other words, the moiety does not block nucleotide or substrate binding, but inhibits kinase activity directly. In another embodiment, the moiety does not cause a conformational change in the RTK kinase domains. In another embodiment, the moiety increases steric constraints between RTK monomers.

In one embodiment, the moiety does not prevent dimerization of the RTK. In another embodiment, the moiety does prevent dimerization of the RTK. In a specific embodiment, the moiety prevents dimerization of cytoplasmic domains of the RTK.

In one embodiment, the RTK is a fibroblast growth factor receptor (FGFR), e.g., fibroblast growth factor receptor 1 (FGFR1), fibroblast growth factor receptor 2 (FGFR2), fibroblast growth factor receptor 3 (FGFR3), or fibroblast growth factor receptor 4 (FGFR4).

In one embodiment, the moiety binds to amino acid residue Arg577 of FGFR1. In another embodiment, the moiety binds to amino acid residue Arg579 of FGFR2. In yet another embodiment, the moiety binds to amino acid residue Arg580 of FGFR2. In another embodiment, the moiety binds to equivalent residues in FGFR3 or FGFR4.

In another embodiment, the moiety binds to amino acid residue Asp519 of FGFR1. In another embodiment, the moiety binds to equivalent amino acid residues in FGFR2, FGFR3 or FGFR4.

In a further embodiment, the moiety binds to an amino acid residue selected from the group consisting of C488, F489, S518, T521, E522, D554, G555, P556, Q574, P587, P579, W691, T695, P702, G703 and P705 of FGFR1. In one embodiment, the moiety binds to an amino acid residue selected from the group consisting of C491, F492, R577, P582, I590, P705, G706 and P708 of FGFR2. In another embodiment, the moiety binds to an amino acid residue selected from the group consisting of C491, F492, N662, G663, R664, L665, P666, V667, K668, W669, R577, R579, R580, P581, P582, E585, Y589, S587, Y588, D589, I590, P705, G706, P708, F713, K724, A726, N727, C728, T729, N730 and E731 of FGFR2. In one embodiment, the moiety binds to an equivalent amino acid residue in FGFR2, FGFR3 or FGFR4. In another embodiment, the moiety binds to at least two, three, four, five or more amino acid residues selected from the group consisting of R577, D519, C488, F489, S518, T521, E522, D554, G555, P556, Q574, P587, P579, W691, T695, P702, G703 and P705 of FGFR1. In yet another embodiment, the moiety binds to at least two, three, four, five or more amino acid residues selected from the group consisting of C491, F492, R577, P582, I590, P705, G706 and P708 of FGFR2. In another embodiment, the moiety binds to at least two, three, four, five or more amino acid residues selected from the group consisting of C491, F492, N662, G663, R664, L665, P666, V667, K668, W669, R577, R579, R580, P581, P582, E585, Y589, S587, Y588, D589, I590, P705, G706, P708, F713, K724, A726, N727, C728, T729, N730 and E731 of FGFR2. In yet another embodiment, the moiety binds to at least two, three, four, five or more equivalent amino acid residues in FGFR2, FGFR3 or FGFR4.

In another embodiment, the moiety binds to a region of the RTK selected from the group consisting of the β1-β2 loop of a monomer of the RTK, the β3-αC loop of a monomer of the RTK, the β4-B5 loop of a monomer of the RTK, the αD-αE loop of a monomer of the RTK, the αF helix of a monomer of the RTK and the αF-αG loop of a monomer of the RTK.

In one embodiment, the moiety binds to a conformational epitope on the RTK. The conformational epitope may be composed of two or more residues in the asymmetric contact interface of the RTK. In another embodiment, the conformational epitope comprises an amino acid residue selected from the group consisting of R577, D519, C488, F489, S518, T521, E522, D554, G555, P556, Q574, P587, P579, W691, T695, P702, G703 and P705. In yet another embodiment, the conformational epitope comprises an amino acid residue selected from the group consisting of C491, F492, R577, P582, I590, P705, G706 and P708 of FGFR2. In a further embodiment, the conformational epitope comprises an amino acid residue selected from the group consisting of C491, F492, N662, G663, R664, L665, P666, V667, K668, W669, R577, R579, R580, P581, P582, E585, Y589, 5587, Y588, D589, I590, P705, G706, P708, F713, K724, A726, N727, C728, T729, N730 and E731 of FGFR2. In yet another embodiment, the conformational epitope comprises an amino acid residue which is an equivalent amino acid residue in FGFR2, FGFR3 or FGFR4.

In another embodiment, the moiety binds to a contiguous epitope on the RTK. In one embodiment, the contiguous epitope is composed of two or more residues in the asymmetric contact interface of the RTK.

In one embodiment, the moiety is a small molecule. In yet another embodiment, the small molecule binds to at least one of the amino acid residues selected from the group consisting of amino acid residue R577, D519, C488, F489, S518, T521, E522, D554, G555, P556, Q574, P587, P579, W691, T695, P702, G703 and P705 of FGFR1. In yet another embodiment, the small molecule binds to at least one of the amino acid residues selected from the group consisting of C491, F492, R577, P582, I590, P705, G706 and P708 of FGFR2. In another embodiment, the small molecule binds to at least one of the amino acid residues selected from the group consisting of C491, F492, N662, G663, R664, L665, P666, V667, K668, W669, R577, R579, R580, P581, P582, E585, Y589, S587, Y588, D589, I590, P705, G706, P708, F713, K724, A726, N727, C728, T729, N730 and E731 of FGFR2. In another embodiment, the small molecule binds to a region selected from the group consisting of the β1-β2 loop of a monomer of the RTK, the β3-αC loop of a monomer of the RTK, the β4-B5 loop of a monomer of the RTK, the αD-αE loop of a monomer of the RTK, the αF helix of a monomer of the RTK and the αF-αG loop of a monomer of the RTK. In one embodiment, the small molecule is designed based on the asymmetric contact interface of a fibroblast growth factor receptor (FGFR).

In another embodiment, the moiety is a peptidic molecule, e.g., a peptidic molecule designed based on the asymmetric contact interface of a fibroblast growth factor receptor (FGFR). In yet another embodiment, the peptidic molecule binds to at least one of the amino acid residues selected from the group consisting of amino acid residue R577, D519, C488, F489, S518, T521, E522, D554, G555, P556, Q574, P587, P579, W691, T695, P702, G703 and P705 of FGFR1. In a further embodiment, the peptidic molecule binds to at least one of the amino acid residues selected from the group consisting of C491, F492, R577, P582, I590, P705, G706 and P708 of FGFR2. In another embodiment, the peptidic molecule binds to at least one amino acid residue selected from the group consisting of C491, F492, N662, G663, R664, L665, P666, V667, K668, W669, R577, R579, R580, P581, P582, E585, Y589, 5587, Y588, D589, 1590, P705, G706, P708, F713, K724, A726, N727, C728, T729, N730 and E731 of FGFR2. In one embodiment, the peptidic molecule binds to a region selected from the group consisting of the β1-β2 loop of a monomer of the RTK, the β3-αC loop of a monomer of the RTK, the β4-B5 loop of a monomer of the RTK, the αD-αE loop of a monomer of the RTK, the αF helix of a monomer of the RTK and the αF-αG loop of a monomer of the RTK. In another embodiment, the peptidic molecule comprises a structure which is at least 80% identical to amino acid residues 576-594 of FGFR1. In another embodiment, the peptidic molecule comprises a structure which is at least 80% identical to amino acid residues 579-597 of FGFR2.

In one embodiment, the moiety is an isolated antibody, or an antigen-binding portion thereof, such as an intrabody. In another embodiment, the antibody, or antigen-binding portion thereof, is selected from the group consisting of a human antibody, a humanized antibody, a bispecific antibody, and a chimeric antibody. In another embodiment, the antibody, or antigen-binding portion thereof, comprises a heavy chain constant region selected from the group consisting of IgG1, IgG2, IgG3, IgG4, IgM, IgA and IgE constant regions. In yet another embodiment, the antibody heavy chain constant region is IgG1. In a further embodiment, the antibody, or antigen-binding portion thereof, is a single chain Fv fragment, an SMIP, an affibody, an avimer, a nanobody, and a single domain antibody. In another embodiment, the antibody, or antigen-binding portion thereof, binds to the asymmetric contact interface of a receptor tyrosine kinase with a KD selected from the group consisting of 1×10⁻⁷ M or less, more preferably 5×10⁻⁸ M or less, more preferably 1×10⁻⁸ M or less, more preferably 5×10⁻⁹ M or less.

In another aspect, the invention provides a hybridoma which produces an antibody, or antigen binding portion thereof, of the invention.

In another aspect, the invention provides a moiety that binds to a conformational epitope on an asymmetric contact interface of a fibroblast growth factor receptor (FGFR), wherein the moiety inhibits ligand induced trans autophosphorylation of the FGFR.

In yet another aspect, the invention provides a moiety that binds to an amino acid residue selected from the group consisting of R577, D519, C488, F489, S518, T521, E522, D554, G555, P556, Q574, P587, P579, W691, T695, P702, G703 and P705 of FGFR1, or within 1-5 Å of said residue, thereby inhibiting ligand induced trans autophosphorylation of FGFR1. In another aspect, the invention provides a moiety that binds to an amino acid residue selected from the group consisting of C491, F492, R577, P582, I590, P705, G706 and P708 of FGFR2, or within 1-5 Å of said residue, thereby inhibiting ligand induced trans autophosphorylation of FGFR2. In another aspect, the invention provides a moiety that binds to an amino acid residue selected from the group consisting of C491, F492, N662, G663, R664, L665, P666, V667, K668, W669, R577, R579, R580, P581, P582, E585, Y589, 5587, Y588, D589, I590, P705, G706, P708, F713, K724, A726, N727, C728, T729, N730 and E731 of FGFR2, or within 1-5 Å of said residue, thereby inhibiting ligand-induced trans autophosphorylation of FGFR2.

In another aspect, the invention provides a moiety that binds to an asymmetric contact interface of a receptor tyrosine kinase (RTK), wherein the moiety disrupts the interface between the N-lobe of an RTK monomer which serves as an enzyme and the C-lobe of an RTK monomer which serves as a substrate.

In yet another aspect, the invention provides a moiety that binds to an asymmetric contact interface of a receptor tyrosine kinase (RTK), wherein the moiety inhibits reverse dephosphorylation of the RTK.

In another aspect, the invention provides a pharmaceutical composition comprising the moiety of the invention and a pharmaceutically acceptable carrier.

In yet another aspect, the invention provides a method of treating or preventing an RTK associated disease in a subject. The method includes administering to the subject an effective amount of the moiety of the invention, thereby treating or preventing the disease in the subject. In one embodiment, the RTK associated disease is selected from the group consisting of cancer and severe bone disorders. In one embodiment, the severe bone disorder is selected from the group consisting of achondroplasia, Crouzon syndrome and Saethre-Chotzen syndrome. In another embodiment, the RTK associated disease is LADD syndrome. In yet another embodiment, the cancer is selected from the group consisting of glioblastoma, multiple myeloma, prostate cancer, pancreatic cancer, bladder cancer and breast cancer.

In another aspect, the invention provides a method for identifying a moiety that binds to an asymmetric contact interface of a receptor tyrosine kinase (RTK) and inhibits ligand-induced trans autophosphorylation of the RTK. The method includes contacting a RTK with a candidate moiety; simultaneously or sequentially contacting the RTK with a ligand for the RTK; determining whether the moiety affects the positioning, orientation and/or distance between the N-lobe of an RTK monomer which functions as an enzyme and the C-lobe of an RTK monomer which functions as a substrate, thereby identifying a moiety that binds to an asymmetric contact interface of the RTK and inhibits ligand-induced trans autophosphorylation of the RTK. In one embodiment, the moiety inhibits ligand induced trans autophosphorylation of the RTK. In another embodiment, the moiety does not cause the loss of intrinsic RTK kinase activity.

In another aspect, the invention provides a small molecule that binds to an asymmetric contact interface of a receptor tyrosine kinase (RTK), wherein the small molecule inhibits trans autophosphorylation of the RTK. In one embodiment, the small molecule binds to an amino acid residue selected from the group consisting of R577, D519, C488, F489, S518, T521, E522, D554, G555, P556, Q574, P587, P579, W691, T695, P702, G703 and P705 of FGFR1, or within 1-5 Å of said residue. In another embodiment, the small molecule binds to an amino acid residue selected from the group consisting of C491, F492, R577, P582, I590, P705, G706 and P708 of FGFR2, or within 1-5 Å of said residue. In another embodiment, the small molecule binds to an amino acid residue selected from the group consisting of C491, F492, N662, G663, R664, L665, P666, V667, K668, W669, R577, R579, R580, P581, P582, E585, Y589, S587, Y588, D589, I590, P705, G706, P708, F713, K724, A726, N727, C728, T729, N730 and E731 of FGFR2, or within 1-5 Å of said residue. In another embodiment, the small molecule binds to a region selected from the group consisting of the β1-β2 loop of a monomer of the RTK, the β3-αC loop of a monomer of the RTK, the β4-B5 loop of a monomer of the RTK, the αD-αE loop of a monomer of the RTK, the αF helix of a monomer of the RTK and the αF-αG loop of a monomer of the RTK.

Other features and advantages of the invention will be apparent from the following detailed description and claims.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 depicts the overall structure of asymmetric activated FGFR1 kinase dimer and detailed views of inter receptor contacts. FIG. 1A depicts an asymmetric dimer of active phosphorylated FGFR1 is shown in ribbon diagram. Molecules E and S of the asymmetric dimer are colored in cyan and green, respectively. FIG. 1B depicts a detailed view of the interface formed between kinases in the asymmetric dimer. ATP analog (AMP-PCP) and interacting residues are shown in stick representation and the magnesium ion is shown as a blue sphere. Residues from molecule S are labeled with primes. The color scheme applied in this figure is used for all figures. Secondary structures are labeled in blue. FIG. 1C depicts the surface representation of molecule E is depicted in cyan with interacting residues of the molecule S in stick and ribbon representation. Representative residues from molecule S are labeled. FIG. 1D depicts the surface representation of molecule S is shown in green with interacting residues of molecule E (pale cyan) in stick and ribbon representation (www.pymol.org).

FIG. 2 depicts the surface distributions of residues in the asymmetric FGFR1 kinase dimer interface. FIG. 1A depicts the overall structures of the asymmetric kinase dimer are shown in ribbon format. FIG. 1B depicts the surface presentation of molecule E (the enzyme) is in cyan. The proximal substrate-binding region is shown in red and distal substrate-binding region is shown in yellow. Activation-loop (A-loop) and nucleotide-binding loop (N-loop) are indicated. FIG. 1C depicts the surface representation of molecule S (substrate) is in green with the tyrosine autophosphorylation site (Y583) in the kinase insert region of molecule S indicated. Substrate site of molecule S is colored in red and the distal substrate site is in yellow.

FIG. 3 demonstrates the autophosphorylation of FGFR1 in vitro and in vivo. Profiles of in vitro phosphorylation reactions of isolated kinase domains of wt-FGFR1 (FIG. 3A) and FGFR1-RE (FIG. 3B) at room temperature as a function of time. FIG. 3C demonstrates that the kinase activity of FGFR1-RE in vitro is maintained. Lysates of L6 cells expressing wt-FGFR1 or the FGFR1-RE mutant were subjected to immunoprecipitation with anti-FGFR1 antibodies. The immunoprecipitates were then incubated in the presence or absence of an FGFR1 substrate (PLCγ fragment, described in the results) for 30 minutes at room temperature followed by SDS-PAGE and immunoblotting with anti-pTyr or anti-FGFR1 antibodies. FIG. 3D demonstrates that autophosphorylation of FGFR1-RE in vivo, is strongly compromised. L6 cells expressing either wt-FGFR1 or its RE mutant were stimulated with increasing concentrations of FGF (as indicated) for 10 minutes at 37° C. Lysates of unstimulated or FGF stimulated cells were subjected to immunoprecipitation using anti-FGFR1 antibodies followed by SDS-PAGE and immunoblotting with anti-pTyr or anti FGFR1 antibodies. FIG. 3E demonstrates that L6 cells expressing wt-FGFR1 or FGFR1-RE were stimulated with 100 ng/ml FGF for different times (as indicated). Lysates of unstimulated or FGF stimulated cells were subjected to SDS-PAGE followed by immunoblotting with anti-pTyr or anti-FGFR1 antibodies.

FIG. 4 depicts the structures of kinase domains of (FIG. 4A) wt-FGFR1 (PDB ID: 3KY2), (FIG. 4B) FGFR1-RE mutant (PDB ID: 3KXX) and (FIG. 4C) activated FGFR1 (FGFR1-3P) (PDB ID: 3GQI) in a simplified cartoon (above) and in a ribbon diagram (below). The catalytic loop is shown in yellow, and the activation loop in green, helix αC is depicted as a cylinder in the cartoon. Phosphotyrosines are colored in red in the cartoon and in stick representation in the ribbon diagram. FIG. 4D depicts ribbon diagrams of kinase insert loops of FGFR1, FGFR1-RE and FGFR1-3P shown in green, cyan and blue, respectively. Side chains of R576, R577 and R577E are shown in stick representation. FIG. 4E depicts the superposition of kinase insert regions of FGFR1 (green), FGFR1-RE (cyan) and FGFR1-3P (blue) revealing multiple conformations of the kinase insert regions in the three structures.

FIG. 5 depicts distances between sequentially ordered FGFR1 tyrosine autophosphorylation sites. A model of FGFR1 (including residue Y766 not yet observed in an FGFR1 structure) is shown in ribbon diagram and six phosphotyrosine sites in stick representation and colored in red. The sequence of autophosphorylation of the six autophosphorylation sites of FGFR1 is marked with numbers and approximate distances between inter autophosphorylation sites shown. Distances between two phosphotyrosine sites are the average of distance between unphosphorylated and phosphorylated FGFR1 structures, and summarized in the table.

FIG. 6 depicts the electron densities around R577 of active FGFR1-3P and R577E of FGFR1-RE. FIG. 6A depicts the electron density of active FGFR1-3P (3GQI) around the kinase insert region. Two kinase domains are shown in cyan and green ribbon representation. D519 of molecule E, and R577′ and Y583E′ of molecule S are shown in stick presentation. AMP-PCP is shown in stick presentation and Mg ion is shown as blue sphere. The 2FoFc electron density map is shown in gray and contoured at 1.0 σ. FIG. 6B depicts an example 2FoFc electron density map of FGFR1-RE is shown with two kinase domains in a ribbon diagram and the side chains of R576 and R577E are shown in stick presentation and contoured at1.0 σ.

FIG. 7 depicts superpositions of all four molecules of FGFR1-RE. The four molecules of FGFR1-RE in the crystal lattice are superimposed and colored in gradient from green to light green.

FIG. 8 depicts the overall structures of asymmetric FGFR1 and FGFR2 kinase dimers are shown in ribbon diagrams (FIGS. 8A and 8B) or as cartoons (FIGS. 8C and 8D). The proximal and distal substrate interfaces are marked by a yellow or a red sphere, respectively. Illustrative representations of the asymmetric FGFR1 or FGFR2 kinase dimers. The phosphorylated regions and activation loops of both structures are shown. Helix αC is shown as a cylinder. The proximal substrate interface of both structures is marked by a yellow circle, and the distal substrate interface is marked by a red circle.

FIG. 9 depicts structure-based alignments of sequences from human FGFR1 and FGFR2 kinases and locations of loss-of-function mutations near helix αG. FIG. 9A demonstrates the degree of residue conservation with either blank (lowest), one dot, two dots or star (highest) for the four FGF receptors at the bottom of FGFR1 and FGFR2 sequences. The overall structure of FGFR1 is shown in gray ribbon, and the corresponding regions in the structure of sequences are indicated with arrows. Green colored residues are from molecule E (the enzyme) and the colored residues are from the molecule S (the substrate). FIG. 9B depicts the location of loss of function mutations in loops near the helix αG of FGFR1 and FGFR2 in blue boxes. Amino acids from molecule E are shown in green, and amino acids from molecule S are in red. Helix αG is marked with the black box with the label on top of the sequences.

FIG. 10 demonstrates the data collection and structure refinement statistics for wt-FGFR1 and the R577E mutant, FGFR1-RE.

FIG. 11 depicts potential regions and amino acid residues involved in the asymmetric contact interface of FGFR1.

FIG. 12 depicts a sequence alignment of fibroblast growth factor receptor sequences (FGFR1, FGFR2, FGFR3 and FGFR4). The boxed sequences indicate sequences responsible for mediating an asymmetric contact interface, and amino acids colored in blue and red are located in the enzyme and substrate molecules, respectively. In one aspect, the invention provides a moiety that binds to any of the boxed amino acid residues of FGFR1, FGFR2, FGFR3 or FGFR4.

### DETAILED DESCRIPTION OF THE INVENTION

The present invention provides moieties, e.g., small molecules, peptidic molecules, aptamers, antibodies or antigen binding portions thereof, and adnectins, that bind to the asymmetric contact interface of a human receptor tyrosine kinase, e.g., an FGFR receptor, such as the human FGFR1, FGFR2, FGFR3 or FGFR4. The moieties of the invention bind to the asymmetric contact interface of the receptor tyrosine kinase and inhibit the trans autophosphorylation of the RTK. In some cases, the moiety does not cause the loss of intrinsic kinase activity in RTK kinase domains. In other words, the moiety may allow dimerization of the RTK, but affect the positioning of the two asymmetric dimers or alter or prevent conformational changes in the receptors, thereby inhibiting the ligand-induced trans autophosphorylation of the RTK. The present invention is based, at least in part, on the deciphering of the crystal structure of the asymmetric contact interface required for the trans autophosphorylation of human FGFR1. The deciphering of this interface has allowed for the identification of residues and, sites and epitopes, e.g., conformational epitopes and contiguous epitopes, which the moieties of the invention may target.

After activation by its ligand, two monomers of a receptor tyrosine kinase interact to form an asymmetric dimer, characterized by an asymmetric contact interface, and subsequently undergo trans autophosphorylation. As used herein, the term “asymmetric contact interface” is intended to include the region of one receptor tyrosine kinase monomer which asymmetrically interacts with a second receptor tyrosine kinase monomer and which is required for trans autophosphorylation of a tyrosine residue of the receptor tyrosine kinase; a step required for tyrosine kinase activation and cell signaling. The two regions of the asymmetric dimer interface are complementary, and the formation of the asymmetric contact interface is required for activation of the receptor tyrosine kinase. Each tyrosine which undergoes trans autophosphorylation on an RTK is associated with a distinct asymmetric contact interface.

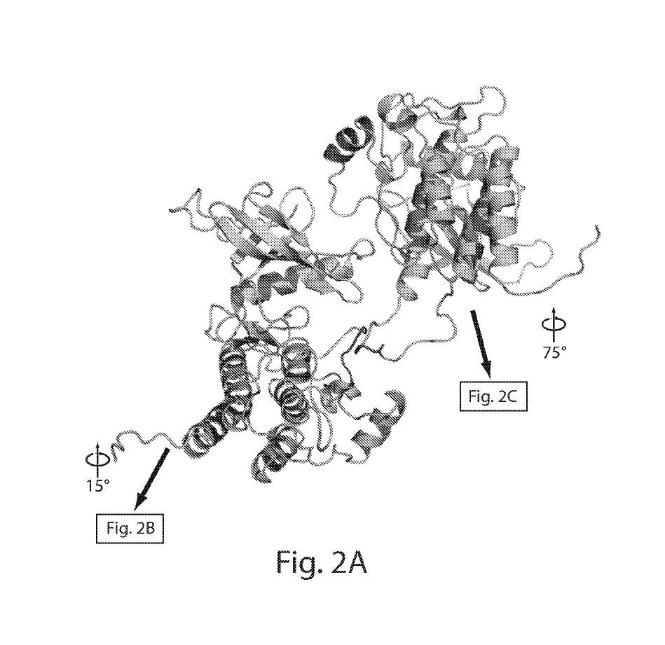

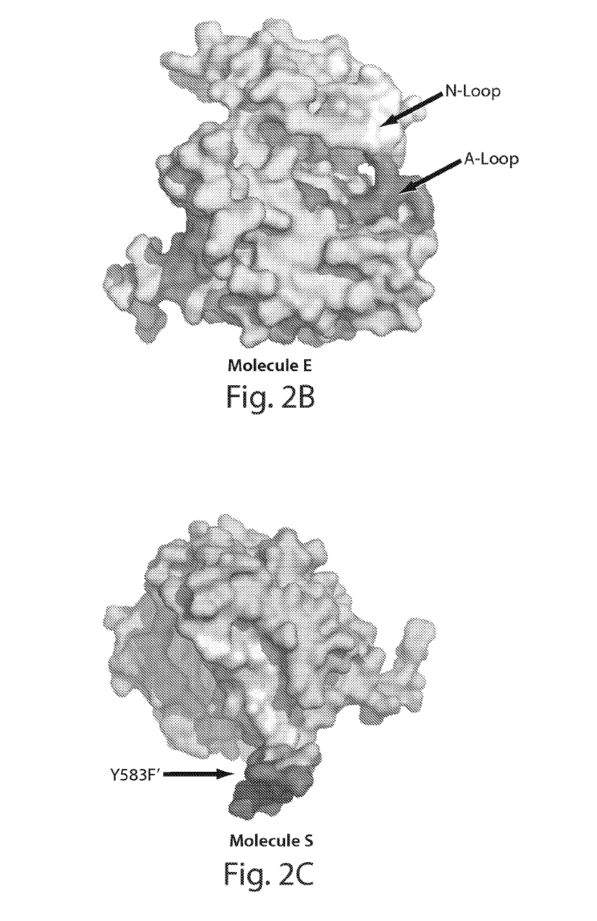

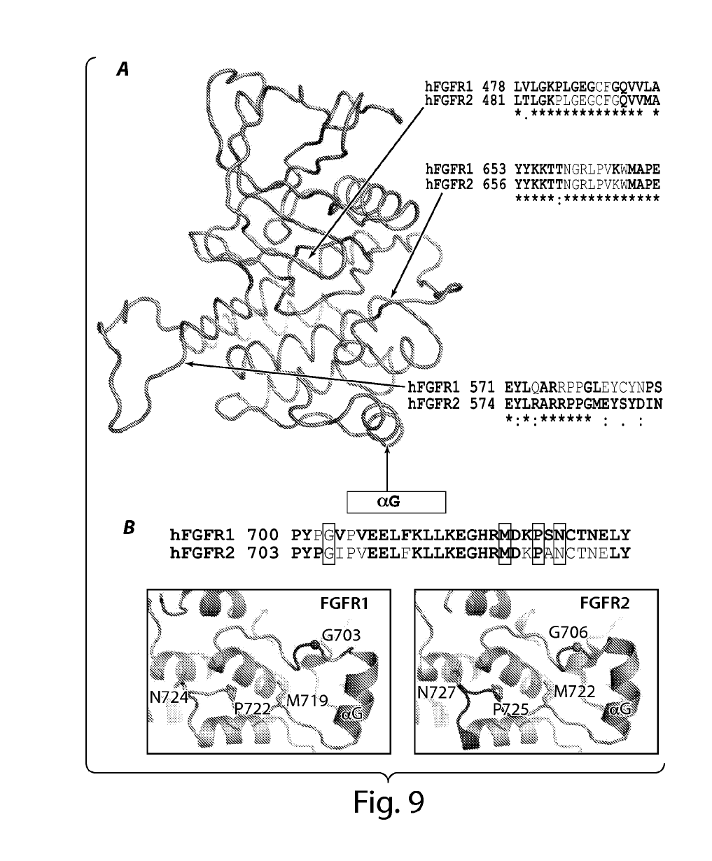

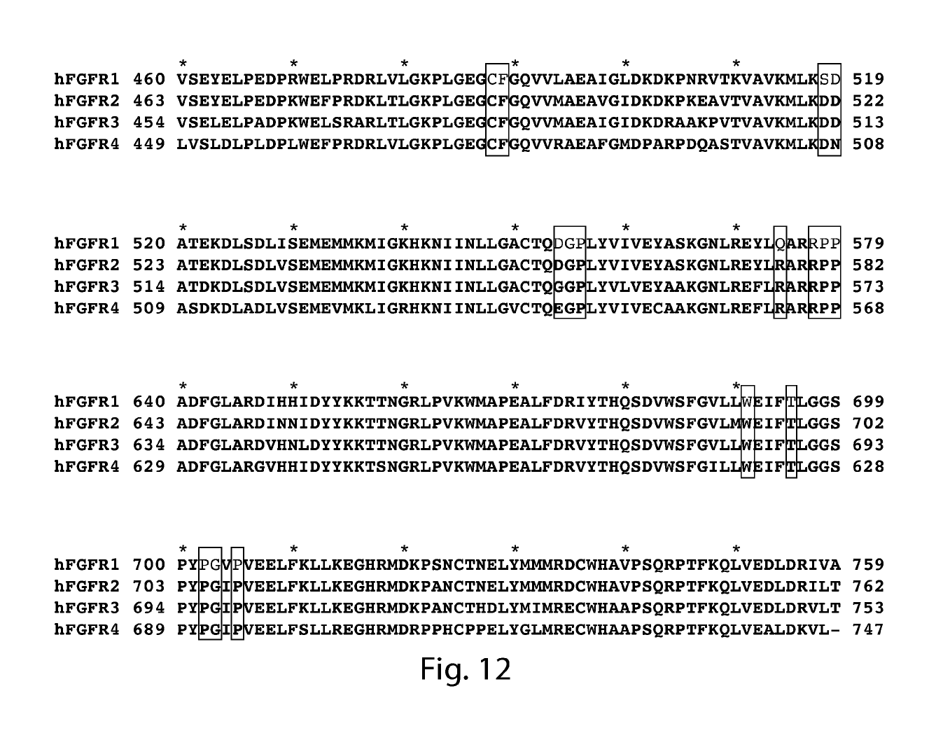

In one embodiment, the asymmetric contact interface of the receptor tyrosine kinase comprises the N-lobe of one receptor tyrosine kinase molecule, such as FGFR1, which serves as an active enzyme. In another embodiment, the asymmetric contact interface of the receptor tyrosine kinase comprises the C-lobe of a receptor tyrosine kinase molecule, such as FGFR1, which serves as a substrate. In one embodiment, the asymmetric contact interface of FGFR1 comprises the yellow region of FGFR1 as shown in FIG. 2. In another embodiment, the asymmetric contact interface of FGFR1 does not comprise the red region of FGFR1 as depicted in FIG. 2. In another embodiment, the asymmetric contact interface of FGFR1 comprises amino acid residue Arg577 of FGFR1. In another embodiment, the asymmetric contact interface of FGFR1 comprises amino acid residue Asp519 of FGFR1. In yet another embodiment, the asymmetric contact interface of FGFR1 comprises the β1-B2 loop, the β3-αC loop, or the β4-β5 loop of the RTK which serves as an enzyme. In another embodiment, the asymmetric contact interface of FGFR1 comprises the αD-αE loop (kinase insert), the αF helix, or the αF-αG loop of the RTK which serves as a substrate. In another embodiment, the asymmetric contact interface of FGFR1 comprises residues C488, F489, S518, D519, T521, E522, D554, G555 or P556 of the RTK which serves as an enzyme. In another embodiment, the asymmetric contact interface of FGFR1 comprises residues Q574, R577, P587, P579, W691, T695, P702, G703 or P705 of the RTK which serves as a substrate. In a specific embodiment, the interaction of the asymmetric contact interfaces of two monomers described herein is required for the trans autophosphorylation of Y583 of FGFR1. In another embodiment, the interaction of the asymmetric contact interfaces of two monomers described herein is required for the trans autophosphorylation of Y653, Y463, Y766, Y585 or Y654 of FGFR1. The structure-based sequence alignments presented in FIGS. 9 and 12 show that the residues involved in asymmetric contact formation are conserved in FGFR1, FGFR2, FGFR3 and FGFR4.

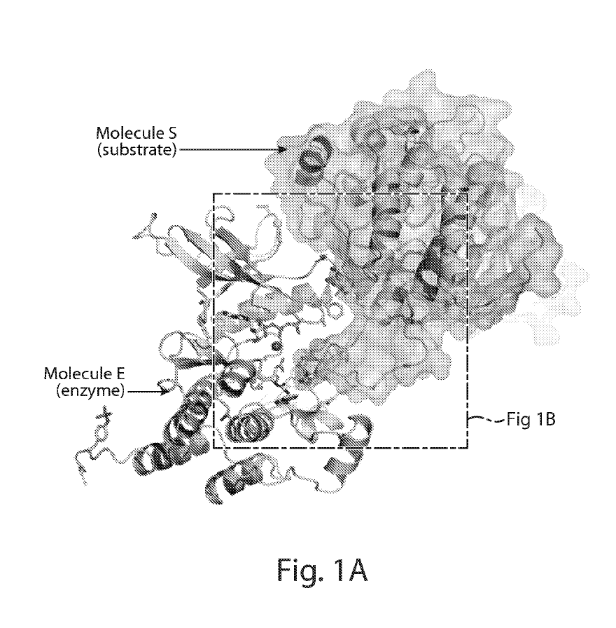

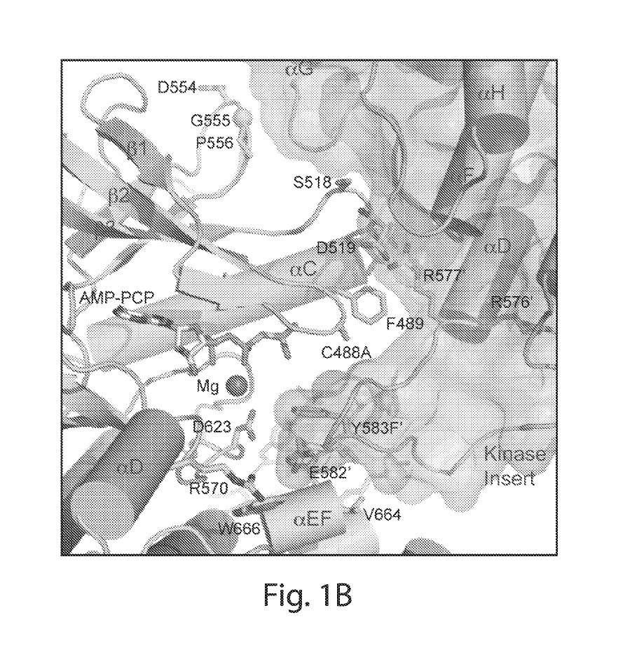

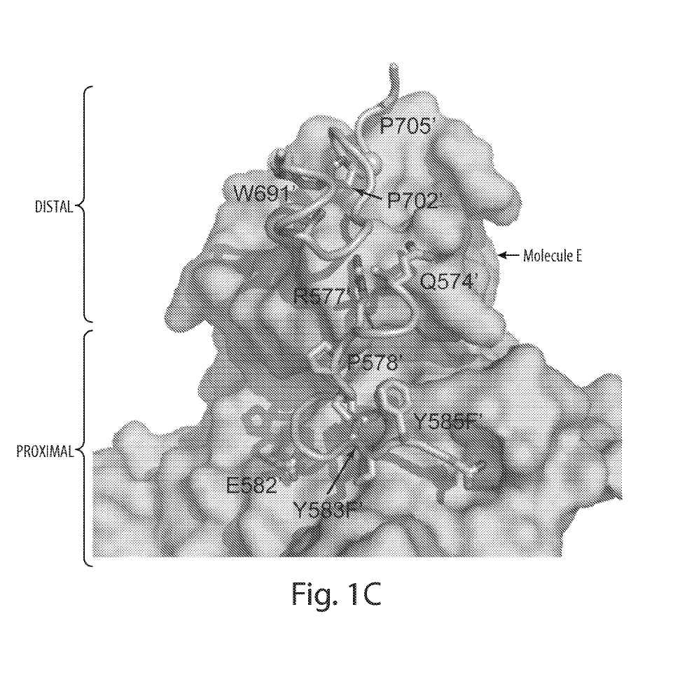

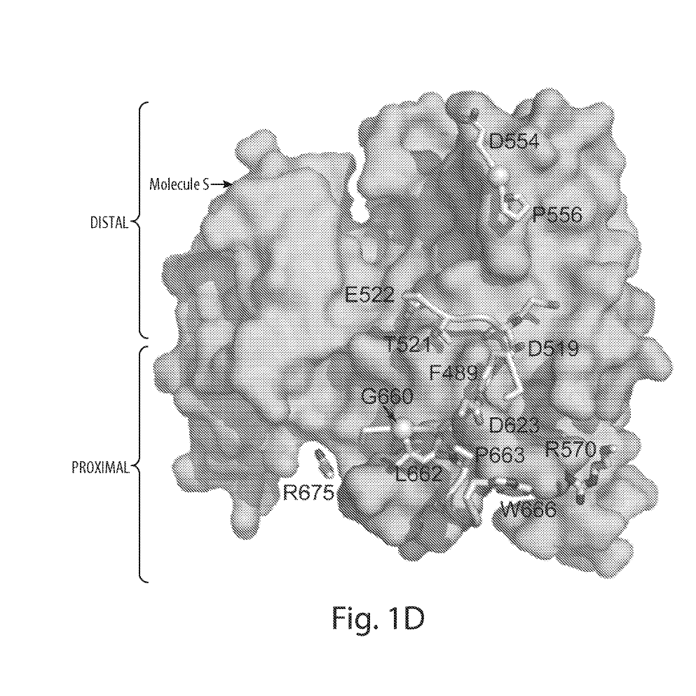

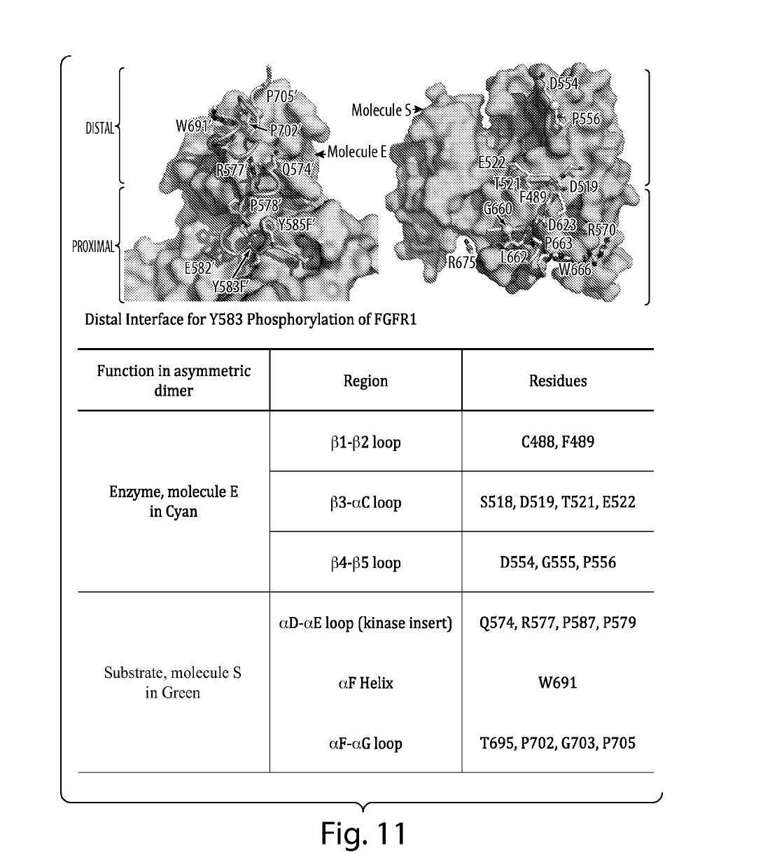

In one embodiment, the asymmetric contact interface is formed between the activation segment, the tip of the nucleotide-binding loop, the β3-αC loop, the β4-β5 loop and the N-terminal region of helix αC in the N-lobe of FGFR1, which serves as an enzyme in the asymmetric FGFR1 dimer. This region interacts with amino acids in helices αF and αG and with N-terminal residues of the kinase insert region located in the C-lobe of a second FGFR1 molecule serving as a substrate in the asymmetric dimer (see FIGS. 1, 2, 8 and 11). As used herein, the term “N-lobe” refers to the portion of the RTK which contains the nucleotide binding site and/or the asymmetric contact interface of the RTK monomer which serves as an enzyme. As used herein, the term “C-lobe” refers to the portion of the RTK which contains the catalytic domain and/or the asymmetric contact interface of the RTK monomer which serves as a substrate.

As used herein, the term “phosphorylation” refers to the addition of a phosphate group to a protein by a kinase. As used herein, the term “autophosphorylation” or “cis phosphorylation” refers to the phosphorylation of a kinase by the kinase protein, itself. As used herein, the term “trans autophosphorylation” refers to the phosphorylation of one monomer of a kinase protein which acts as a substrate by the other monomer of a kinase protein which acts as an enzyme in a dimerized receptor. Ligand-induced trans autophosphorylation plays an important role in the control of activation and cell signaling by RTKs. As used herein, the term “ligand-induced trans autophosphorylation” refers to the activation of trans autophosphorylation of a receptor tyrosine kinase upon binding of the RTK to its ligand.

As used herein, the term “moiety” is intended to include any moiety binds to the asymmetric contact interface of a receptor tyrosine kinase, where the moiety inhibits the trans autophosphorylation of the RTK. The moiety can be a small molecule; a peptidic molecule (e.g., a peptidic molecule designed based on the structure of an asymmetric contact interface of a receptor tyrosine kinase); an isolated antibody, or antigen binding portion thereof; an aptamer or an adnectin.

In some embodiments, the moiety will bind to specific sequences in the asymmetric contact interface of the human receptor tyrosine kinase. In specific embodiments, the moiety will bind to specific sequences in the asymmetric contact interface of an FGFR receptor, for example, residues R577, D519, C488, F489, S518, T521, E522, D554, G555, P556, Q574, P587, P579, W691, T695, P702, G703 or P705 of FGFR1. These residues within the β1-β2 loop of one monomer of the RTK, the β3-αC loop of one monomer of the RTK, the β4-B5 loop of one monomer of the RTK, the αD-αE loop of one monomer of the RTK, the αF helix of one monomer of the RTK and the αF-αG loop of one monomer of the RTK comprise the asymmetric contact interface domain of the human FGFR1 and are shown herein to be critical to trans autophosphorylation of the receptor. One of skill in the art will appreciate that, in some embodiments, moieties of the invention may be easily targeted to the corresponding residues in other RTKs, e.g., those residues that form similar pockets or cavities of an asymmetric contact interface or those in the same position by structural alignment or sequence alignment. One of skill in the art will appreciate that a moiety which specifically binds to the aforementioned residues (or within a certain distance, e.g., within 1 2, 3, 4 or 5 Å from those residues) in the asymmetric contact interface can antagonize the activity of the receptor by, for example, preventing trans autophosphorylation of the RTK.

Thus, in some embodiments, a moiety of the invention may bind to contiguous or non-contiguous amino acid residues and function as a molecular wedge that prevents the motion required for positioning of the asymmetric contact interface of the RTK at a distance and orientation that enables trans autophosphorylation of the RTK.

In a specific embodiment, a moiety of the invention binds to a conformational epitope or a discontinuous epitope on a RTK. As used herein, the term “epitope” is intended to include residues, motifs, sites or domains of an RTK to which a small molecule, antibody or antigen-binding fragment thereof, or peptidic molecule of the invention may bind. The conformational or discontinuous epitope may be composed of two or more residues from the asymmetric contact interface of an RTK, e.g., the human FGFR1, FGFR2, FGFR3 or FGFR4. In a particular embodiment, a moiety of the invention binds to a conformational epitope composed of 2 or more amino acids selected from the group consisting of R577, D519, C488, F489, S518, T521, E522, D554, G555, P556, Q574, P587, P579, W691, T695, P702, G703 and P705 of FGFR1. In another embodiment, a moiety of the invention binds to a conformational epitope composed of 3 or more amino acids selected from the group consisting of R577, D519, C488, F489, S518, T521, E522, D554, G555, P556, Q574, P587, P579, W691, T695, P702, G703 and P705 of FGFR1. In another embodiment, a moiety of the invention binds to a conformational epitope composed of 4 or more amino acids selected from the group consisting of R577, D519, C488, F489, S518, T521, E522, D554, G555, P556, Q574, P587, P579, W691, T695, P702, G703 and P705 of FGFR1. In another embodiment, a moiety of the invention binds to a conformational epitope composed of 5 or more amino acids selected from the group consisting of R577, D519, C488, F489, S518, T521, E522, D554, G555, P556, Q574, P587, P579, W691, T695, P702, G703 and P705 of FGFR1. As indicated above, the moieties of the invention may bind to all of the amino acid residues forming a pocket or a cavity of an asymmetric contact interface or they may bind to a subset of the residues forming the asymmetric contact interface. It is to be understood that, in certain embodiments, when reference is made to a moiety of the invention binding to an epitope, e.g., a conformational epitope, the intention is for the moiety to bind only to those specific residues that make up the epitope and not other residues in the linear amino acid sequence of the receptor.

In another embodiment, a moiety of the invention binds to a contiguous epitope on the RTK. In one embodiment, the contiguous epitope is composed of two or more residues in the asymmetric contact interface of the FGFR. In another embodiment, the contiguous epitope is an epitope selected from the group consisting of the β1-β2 loop of a monomer of the RTK, the β3-αC loop of a monomer of the RTK, the β4-B5 loop of a monomer of the RTK, the αD-αE loop of a monomer of the RTK, the αF helix of a monomer of the RTK and the αF-αG loop of a monomer of the RTK.

Moieties of the invention may exert their inhibitory effect on receptor activation by preventing critical homotypic interactions (such as salt bridges) between asymmetric contact interfaces of RTKs that are essential for positioning RTK monomers at a distance and orientation essential for tyrosine kinase activation. Thus, moieties of the invention may allow dimerization of the RTK while preventing trans autophosphorylation. It will also be appreciated by one of skill in the art that a moiety of the invention may bind to sugar residues which may appear on a glycosylated form of an RTK. It is further possible that a moiety of the invention will bind an epitope that is composed of both amino acid residues and sugar residues.

The terms “receptor tyrosine kinase” and “RTK” are used interchangeably herein to refer to the well known family of membrane receptors that phosphorylate tyrosine residues. Many play significant roles in development or cell division. Receptor tyrosine kinases possess an extracellular ligand binding domain, a transmembrane domain and an intracellular catalytic domain. The extracellular domains bind cytokines, growth factors or other ligands and are generally comprised of one or more identifiable structural motifs, including cysteine-rich regions, fibronectin III-like domains, immunoglobulin-like domains, EGF-like domains, cadherin-like domains, kringle-like domains, Factor VIII-like domains, glycine-rich regions, leucine-rich regions, acidic regions and discoidin-like domains. Activation of the intracellular kinase domain is achieved by ligand binding to the extracellular domain, which induces dimerization of the receptors. A receptor activated in this way is able to autophosphorylate tyrosine residues outside the catalytic domain, facilitating stabilization of the active receptor conformation. The phosphorylated residues also serve as binding sites for proteins which will then transduce signals within the cell. Examples of RTKs include, but are not limited to, Kit receptor (also known as Stem Cell Factor receptor or SCF receptor), fibroblast growth factor (FGF) receptors, hepatocyte growth factor (HGF) receptors, insulin receptor, insulin-like growth factor-1 (IGF-1) receptor, nerve growth factor (NGF) receptor, vascular endothelial growth factor (VEGF) receptors, PDGF-receptor-α, PDGF-receptor-β, CSF-1-receptor (also known as M-CSF-receptor or Fms), and the Flt3-receptor (also known as Flk2).

In a preferred embodiment of the invention, the RTK is a type IV RTK. In another embodiment of the invention, the RTK is a type V RTK, i.e., a member of the VEGF receptor family, or a type III RTK, i.e., a member of the PDGF receptor family.

As used herein, the term “type IV family of receptor tyrosine kinases” or “type IV RTK” is intended to include receptor tyrosine kinases which typically comprise three immunoglobulin-like domains (or Ig-like domains), a single transmembrane helix domain, and an intracellular domain with tyrosine kinase activity. The type IV family of RTKs bind fibroblast growth factors. Examples of type IV RTKs include, but are not limited to, FGFR1, FGFR2, FGFR3 and FGFR4.

As used herein the term “type III family of receptor tyrosine kinases” or “type III RTKs” is intended to include receptor tyrosine kinases which typically contain five immunoglobulin like domains, or Ig-like domains, in their ectodomains. Examples of type III RTKs include, but are not limited to PDGF receptors, the M-CSF receptor, the FGF receptor, the Flt3-receptor (also known as Flk2) and the Kit receptor. In a preferred embodiment of the invention, the type III RTK is Kit (also known in the art as the SCF receptor). Kit, like other type III RTKs is composed of a glycosylated extracellular ligand binding domain (ectodomain) that is connected to a cytoplasmic region by means of a single transmembrane (TM) domain (reviewed in Schlessinger (2000) Cell 103: 211-225). Another hallmark of the type III RTKs, e.g., Kit or PDGFR, is a cytoplasmic protein tyrosine kinase (PTK) domain with a large kinase-insert region. At least two splice isoforms of the Kit receptor are known to exist, the shorter making use of an in-frame splice site. All isoforms of Kit, and the other above described RTKs, are encompassed by the present invention.

As used herein, an “Ig-like domain” of a receptor tyrosine kinase (RTK) is intended to include the domains well known in the art to be present in the ectodomain of RTKs. In the ectodomain of the family of type IV receptor tyrosine kinases, there are three such domains. In the ectodomain of the family of type III receptor tyrosine kinases (type III RTKs), e.g., Kit, there are five such domains, known as D1, D2, D3, D4 and D5. The D1, D2 and D3 domains of type III RTKs are responsible for binding the ligand of the RTK (reviewed in Ullrich and Schlessinger (1990) Cell 61: 203-212). Thus, in one embodiment of the invention the term “Ig-like domain” is not intended to include a domain of a RTK which is responsible for ligand binding. In a one embodiment of the invention, the Ig-like domain is a D4 and/or a D5 domain of a type III RTK. In the ectodomain of the VEGF receptor family, there are seven Ig-like domains, known as D1, D2, D3, D4, D5, D6 and D7. In one embodiment of the invention, the Ig-like domain is a D7 domain of the VEGF receptor family.

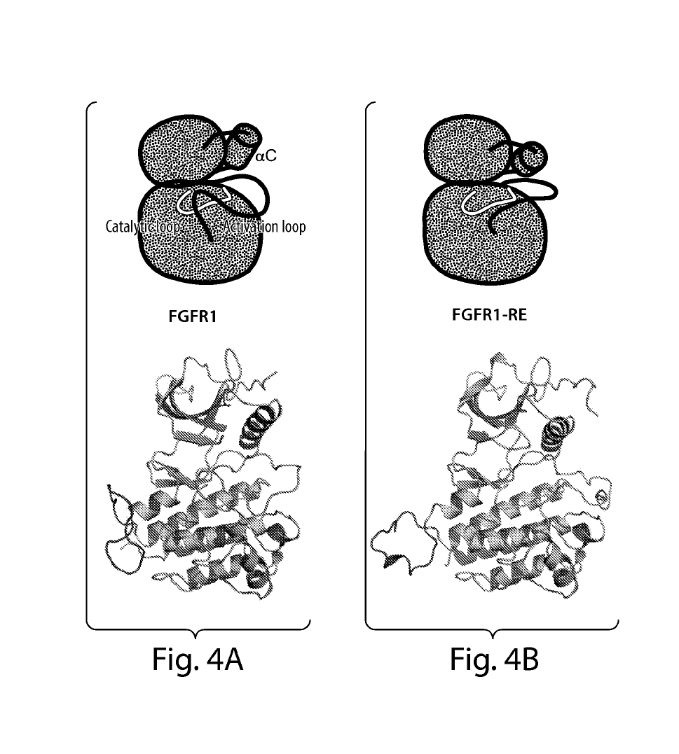

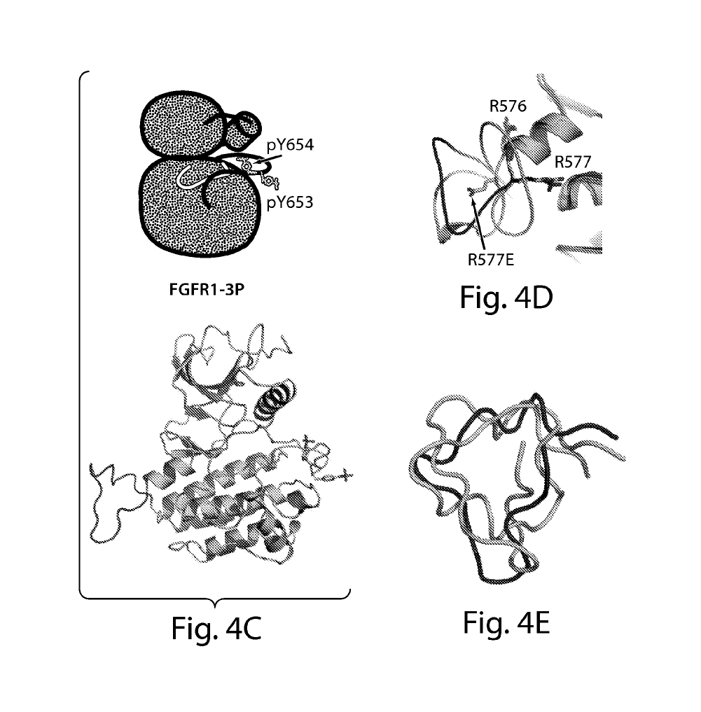

As used herein, the term “catalytic domain” is intended to include the region of an enzyme molecule where catalysis of a substrate occurs. For example, the catalytic domain of RTKs comprises residues of the RTK monomer which acts as an enzyme that are involved in the binding and trans autophosphorylation of the RTK monomer which acts as a substrate. In a specific embodiment of the invention, the catalytic domain comprises the red area of FGFR1 shown in FIG. 2. In one embodiment, the catalytic domain comprises the yellow area of FGFR1 shown in FIG. 4.

As used herein, the phrase “acts as a substrate” or “substrate molecule” is intended to include the receptor tyrosine kinase monomer which is part of a dimerized RTK and which is phosphorylated by another receptor tyrosine kinase monomer (which is part of the dimerized RTK and which acts as the enzyme). As used herein, the phrase “acts as an enzyme” or “enzyme molecule” is intended to include the RTK monomer which is part of a dimerized receptor tyrosine kinase and which acts to enzymatically phosphorylate the other RTK monomer which acts as a substrate.

Similarly, the term “monomer”, as used herein, refers to an RTK molecule which is a single polypeptide chain which is not associated with a second RTK polypeptide of the same or different type. The term “dimer”, as used herein, refers to a molecule comprising two RTK monomers. The term “dimerization”, as used herein, refers to the formation of a dimer molecule comprising two RTK monomers.

The term “monomeric state” as used herein, refers to the state of a RTK wherein the RTK molecule is composed of a single polypeptide chain which is not associated with a second RTK polypeptide of the same or different type. RTK dimerization leads to trans autophosphorylation and receptor activation. Thus, a RTK in a monomeric state is in an inactive state. A monomeric state is also a state wherein the asymmetric contact interface of a single RTK is not associated with the asymmetric contact interface, respectively, of a second, RTK.

The term “ectodomain” of a receptor tyrosine kinase (RTK) is well known in the art and refers to the extracellular part of the RTK, i.e., the part of the RTK that is outside of the plasma membrane.

As used herein, the term “fibroblast growth factor receptor”, “FGFR”, “FGF receptor” or “FGFR family”, also known as type IV RTKs, includes RTK receptors which bind fibroblast growth factors. As described above, these RTKs have three Ig-like domains in their ectodomains. Examples of FGFR family receptors are FGFR1, FGFR2, FGFR3 and FGFR4.

As used herein the term “vascular endothelial growth factor receptor”, “VEGF receptor”, or “VEGF receptor family”, also known as type V RTKs includes RTK receptors for the vascular endothelial growth factor. As described above, these RTKs have 71 g-like domains in their ectodomains. Examples of VEGF family receptors are VEGFR1 (also known as Flt-1), VEGFR2 (also known as KDR or Flk-1), and VEGFR3 (also known as Flt-4).

The term “a membrane proximal region” of the ectodomain of a receptor tyrosine kinase refers to an extracellular part of a RTK which is in proximity to the plasma membrane and which, preferably, is not directly responsible for the binding of a ligand to the RTK. Examples of membrane proximal regions include, but are not limited to, the D4 domain of a type III receptor tyrosine kinase, the D5 domain of a type III receptor tyrosine kinase, the D3-D4 hinge region of a type III receptor tyrosine kinase, the D4-D5 hinge region of a type III receptor tyrosine kinase, and the D7 domain of a type V receptor tyrosine kinase.

The term “homotypic interaction” as used herein, refers to the interaction between two identical regions from two monomeric receptors.

The term “heterotypic interaction” as used herein, refers to the interaction between two different regions from two monomeric receptors. A heterotypic interaction may be the result of dimerization of two different types of monomeric receptors or the result of dimerization of a wild type and a mutant form of the same monomeric receptor. For example, it is well known in the art that a cancer patient may carry a wild type allele and a mutant allele for a certain receptor.

As used herein, a “protomer” is a structural unit of an oligomeric protein, such as an RTK. A protomer is a protein subunit which may assemble in a defined stoichiometry to form an oligomer. The VEGFR family of receptor tyrosine kinases are covalently linked homodimers, and each VEGFR protomer is composed of four stranded β-sheets arranged in an anti-parallel fashion in a structure designated “cysteine-knot growth factors”.

The phrase “inhibits ligand-induced trans autophosphorylation” refers to the ability of a moiety of the invention to inhibit the activity of the receptor tyrosine kinase. In other words, this phrase includes the ability of a moiety of the invention to shift the equilibrium towards formation of an inactive or unphosphorylated receptor configuration. For example, a moiety of the invention may inhibit the trans autophosphorylation of a receptor tyrosine kinase by at least 5%, 10%, 15%, 20%, 25%, 30%, 35%, 40%, 45%, 50%, 55%, 60%, 65%, 70%, 75%, 80%, 85%, 90% or 95% as compared to the activity of the receptor in the absence of the moiety. In some embodiments, a moiety of the invention may inhibit the trans autophosphorylation of all of the tyrosine residues of the RTK. For example, the moiety may inhibit the trans autophosphorylation of Y653, Y583, Y463, Y766, Y585 and Y654 of FGFR1. In another embodiment, a moiety of the invention may inhibit the trans autophosphorylation of all of the tyrosine residues located in the activation loop of the catalytic core of the RTK. For example, the moiety may inhibit the trans autophosphorylation of residue Y653 in the activation loop of the catalytic core of FGFR1. In other embodiments, a moiety of the invention may inhibit the trans autophosphorylation of all of the tyrosine residues located in the kinase insert region. For example, the moiety may inhibit the trans autophosphorylation of residue Y583 and Y585 in the kinase insert region of FGFR1. Alternatively, the moiety may inhibit the trans autophosphorylation of residue Y583 in the kinase insert region of FGFR1, or the moiety may inhibit the trans autophosphorylation of residue Y585 in the kinase insert region of FGFR1. In other embodiments, a moiety of the invention may inhibit the trans autophosphorylation of all of the tyrosine residues located in the juxtamembrane region of the RTK. For example, the moiety may inhibit the trans autophosphorylation of residue Y463 in the juxtamembrane region of FGFR1. In other embodiments, a moiety of the invention may inhibit the trans autophosphorylation of all of the tyrosine residues located in the C-terminal tail of the RTK. For example, the moiety may inhibit the trans autophosphorylation of residue Y766 in the C-terminal tail of FGFR1. In other embodiments, a moiety of the invention may inhibit the trans autophosphorylation of all of the tyrosine residues located in the activation loop of the RTK. For example, the moiety may inhibit the trans autophosphorylation of residue Y654 in the activation loop of FGFR1.

Trans autophosphorylation of all tyrosine autophosphorylation sites is required for full RTK activation and that the trans autophosphorylation of tyrosine residues occurs in a specific order. Accordingly, in another embodiment, a moiety of the invention may inhibit the trans autophosphorylation of one tyrosine residue and any tyrosine residues which would be subsequently trans autophosphorylated. For example, in one embodiment, the moiety may inhibit the trans autophosphorylation of residues Y583, Y463, Y766, Y585 and Y654. In another embodiment, the moiety may inhibit the trans autophosphorylation of residues Y463, Y766, Y585 and Y654. In another embodiment, the moiety may inhibit the trans autophosphorylation of residues Y766, Y585 and Y654. In another embodiment, the moiety may inhibit the trans autophosphorylation of residues Y585 and Y654. In yet another embodiment, the moiety may inhibit the trans autophosphorylation of residue Y654.

The term “inactive state,” as used herein, refers to the state of a RTK wherein the RTK molecule is unable to activate downstream signaling. An inactive state may be a state wherein the receptor tyrosine kinase is allowed to dimerize but the positioning, orientation, conformation, and/or distance between the asymmetric contact interface domains of the two monomers (e.g., asymmetric contact interface of a type IV receptor tyrosine kinase), is altered such that the activity of the receptor tyrosine kinase is inhibited (e.g., tyrosine trans autophosphorylation of the receptor is inhibited and/or the ability of the receptor to activate a downstream signaling pathway is inhibited). An inactive state also includes a monomeric state, as described above. The Examples further discuss experiments which demonstrate that there are specific conserved amino acid residues which are crucial for RTK ligand-induced trans autophosphorylation but which are dispensable for receptor dimerization. The term “inactive state” includes a state in which a moiety of the invention may reduce or inhibit the ligand-induced trans autophosphorylation of a receptor tyrosine kinase by at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% as compared to the trans autophosphorylation of the receptor in the absence of the moiety. Any of the functional assays described herein may be used to determine the ability of a moiety of the invention to inhibit the trans autophosphorylation of a receptor tyrosine kinase. In some embodiments, a moiety of the invention may exhibit a broad effect, e.g., when most or all target RTKs in a subject are inactivated. In other embodiments, a moiety of the invention may exhibit a narrower effect, e.g., when a portion of the target RTKs in a subject are inactivated. In such embodiments, at least 5%, 10%, 15%, 20%, 25%, 30%, 35%, 40%, 45%, 50%, 55%, 60%, 65%, 70%, 75%, 80%, 85%, 90% or 95% of the receptors are locked into an inactive state as compared to the receptors in the absence of said moiety.

As used herein, the terms “conformational epitope” or “non-linear epitope” or “discontinuous epitope” are used interchangeably to refer to an epitope which is composed of at least two amino acids which are not consecutive amino acids in a single protein chain. For example, a conformational epitope may be comprised of two or more amino acids which are separated by a stretch of intervening amino acids but which are close enough to be recognized by a moiety of the invention as a single epitope. As a further example, amino acids which are separated by intervening amino acids on a single protein chain, or amino acids which exist on separate protein chains, may be brought into proximity due to the conformational shape of a protein structure or complex to become a conformational epitope which may be bound by a moiety of the invention. Particular discontinuous and conformation epitopes are described herein.

It will be appreciated by one of skill in the art that, in general, a linear epitope bound by a moiety of the invention may or may not be dependent on the secondary, tertiary, or quaternary structure of the RTK. For example, in some embodiments, a moiety of the invention may bind to a group of amino acids regardless of whether they are folded in a natural three dimensional protein structure. In other embodiments, a moiety of the invention may not recognize the individual amino acid residues making up the epitope, and may require a particular conformation (bend, twist, turn or fold) in order to recognize and bind the epitope.

As used herein, the terms “contiguous epitope” or “continuous epitope” are used interchangeably to refer to an epitope which is composed of at least two amino acids which are consecutive amino acids in a single protein chain. Particular contiguous epitopes are described herein. In one embodiment, the moiety of the invention binds to a contiguous epitope on an RTK. In another embodiment, the contiguous epitope is composed of two or more residues in the asymmetric contact interface of an RTK.

As used herein, the phrase “hydrophobic amino acid” refers to an amino acid comprising hydrophobic properties e.g., alanine, cysteine, phenylalanine, glycine, histidine, isoleucine, lysine, leucine, methionine, arginine, threonine, valine, tryptophan, tyrosine, serine, proline and others listed herein.

Various aspects of the invention are described in further detail in the following subsections:

I. Small Molecules which Bind to an Asymmetric Contact Interface of a Human Receptor Tyrosine Kinase

In one aspect of the invention, the moiety that binds to the asymmetric contact interface of a human receptor tyrosine kinase is a small molecule.

The small molecules of the instant invention are characterized by particular functional features or properties. For example, the small molecules bind to specific residues or regions of an asymmetric contact interface of a RTK. In preferred embodiments, the binding of small molecule inhibitors to the asymmetric contact interface will prevent the movement that enables the RTK monomers to be at a distance and orientation (position) that allows trans-autophosphorylation and activation of the tyrosine kinase domain followed by recruitment and activation of downstream signaling pathways. The small molecule binding may, in some embodiments, allow the receptor tyrosine kinase to dimerize but affect the positioning, orientation and/or distance between the asymmetric contact interface domains of the two monomers (e.g., the asymmetric contact interfaces of a type IV receptor tyrosine kinase), thereby inhibiting ligand-induced trans autophosphorylation and the activity of the receptor tyrosine kinase.

The terms “small molecule compounds”, “small molecule drugs”, “small molecules”, or “small molecule inhibitors” are used interchangeably herein to refer to the compounds of the present invention which are able to inhibit the ligand-induced trans autophosphorylation or activity of the RTK, e.g., an FGFR, such as FGFR1, FGFR2, FGFR3 or FGFR4. These compounds may comprise compounds in PubChem database (pubchem.ncbi.nlm.nih.gov/), the Molecular Libraries Screening Center Network (MLSCN) database, compounds in related databases, or derivatives and/or functional analogues thereof.

As used herein, “analogue” or “functional analogue” refers to a chemical compound or small molecule inhibitor that is structurally similar to a parent compound, but differs slightly in composition (e.g., one or more atoms or functional groups are added, removed, or modified). The analogue may or may not have different chemical or physical properties than the original compound and may or may not have improved biological and/or chemical activity. For example, the analogue may be more hydrophobic or it may have altered activity (increased, decreased, or identical to parent compound) as compared to the parent compound. The analogue may be a naturally or non-naturally occurring (e.g., recombinant) variant of the original compound. Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

As used herein, “derivative” refers to a chemically or biologically modified version of a chemical compound or small molecule inhibitor that is structurally similar to a parent compound and (actually or theoretically) derivable from that parent compound. A “derivative” differs from an “analogue” or “functional analogue” in that a parent compound may be the starting material to generate a “derivative,” whereas the parent compound may not necessarily be used as the starting material to generate an “analogue” or “functional analogue.” A derivative may or may not have different chemical or physical properties of the parent compound. For example, the derivative may be more hydrophilic or it may have altered reactivity as compared to the parent compound. Derivatization (i.e., modification by chemical or other means) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; and H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups such as carboxylic acid groups can form alkali metal salts or alkaline earth metal salts (e.g., sodium salts, potassium salts, magnesium salts, calcium salts, and salts with physiologically tolerable quaternary ammonium ions and acid addition salts with ammonia and physiologically tolerable organic amines such as triethylamine, ethanolamine, or tris-(2-hydroxyethyl)amine). Basic groups can form acid addition salts, for example with inorganic acids such as hydrochloric acid (“HCl”), sulfuric acid, or phosphoric acid, or with organic carboxylic acids and sulfonic acids such as acetic acid, citric acid, benzoic acid, maleic acid, fumaric acid, tartaric acid, methanesulfonic acid, or p-toluenesulfonic acid. Compounds which simultaneously contain a basic group and an acidic group such as a carboxyl group in addition to basic nitrogen atoms can be present as zwitterions. Salts can be obtained by customary methods known to those skilled in the art, for example by combining a compound with an inorganic or organic acid or base in a solvent or diluent, or from other salts by cation exchange or anion exchange.

Small molecules are known to have molecular weights of 1200 or below, 1000 or below, 900 or below, 800 or below, 700 or below, 600 or below, 500 or below, 400 or below, 300 or below, 200 or below, 100 or below, 50 or below, 25 or below, or 10 or below.

The small molecule inhibitors of the present invention are selected or designed to bind to the asymmetric contact interface of an RTK, e.g., a type IV RTK, e.g., a FGFR. In some embodiments, the small molecule inhibitors are selected or designed to bind an asymmetric contact interface of human FGFR1, human FGFR2, human FGFR3, or human FGFR4, thereby inhibiting the ability of the receptor to trans autophosphorylate and become active, e.g., activate an intracellular signal transduction pathway. In other embodiments the small molecule inhibitors are selected to bind domains sharing homology to an asymmetric contact interface of the FGF receptor. For example, a small molecule of the present invention may be directed toward a domain which is at least 50% identical, at least 60% identical, at least 70% identical, at least 80% identical, at least 90% identical, or at least 95% or 99% identical to an asymmetric contact interface of a RTK, e.g., the asymmetric contact interface of FGFR1. Such a small molecule would be capable of binding protein domains which are functionally similar to, for example, the asymmetric contact interface of the FGFR.

The small molecule inhibitors of the present invention may also bind to a particular motif or consensus sequence derived from an asymmetric contact interface of a RTK, e.g., a human FGFR or a human type IV RTK, allowing the small molecule inhibitors to specifically bind domains which are shared among members of the RTK family, e.g., members of the human type IV family of RTKs.

In other embodiments, small molecule inhibitors bind protein motifs or consensus sequences which represent the three dimensional structure of the protein. Such motifs or consensus sequences would not represent a contiguous string of amino acids, but a non-linear amino acid arrangement that results from the three-dimensional folding of the RTK (i.e., a structural motif). An example of such a motif would be a motif designed based on the linear regions of the asymmetric contact interface of the FGF receptor. Such motifs and consensus sequences may be designed according to the methods discussed in the Section regarding antibodies.

Importantly, a small molecule inhibitor of the invention does not bind to the nucleotide binding site of the catalytic domain of a fibroblast growth factor receptor.

In another embodiment, the small molecule inhibitor of the invention binds to a contiguous epitope on the RTK. As used herein, the term “epitope” is intended to include residues, motifs, sites or domains of an RTK to which a small molecule may bind. In one embodiment, the contiguous epitope is composed of two or more residues in the asymmetric contact interface of the RTK. In another embodiment, the contiguous epitope is an epitope selected from the group consisting of the β1-β2 loop of a monomer of the RTK, the β3-αC loop of a monomer of the RTK, the β4-B5 loop of a monomer of the RTK, the αD-αE loop of a monomer of the RTK, the αF helix of a monomer of the RTK and the αF-αG loop of a monomer of the RTK.

In additional embodiments, small molecule inhibitors of the invention are selected or designed to bind specifically to a mutant asymmetric contact interface of a RTK. In preferred embodiments, the mutant RTK is a tumorigenic or an oncogenic mutant. In one specific embodiment, the small molecule inhibitor is selected or designed to bind to an oncogenic FGFR mutant. RTK mutants which may be targeted by the small molecules of the instant invention include, but are not limited to, fibroblast growth factor receptors with mutations in one or more of the following amino acids: R577, D519, C488, F489, S518, T521, E522, D554, G555, P556, Q574, P587, P579, W691, T695, P702, G703 or P705 of FGFR1. It should be appreciated by one of skill in the art that the methods of the invention would be applicable to other mutations in FGFRs or to mutations in other RTKs. One advantage of targeting a mutant RTK is that a therapeutic small molecule may bind to only the RTKs on cells containing the mutation, leaving healthy cells largely or entirely unaffected. Accordingly, in instances where the mutation is tumorigenic, only tumor cells would be targeted for therapy, potentially reducing side effects and dosage requirements.

In some embodiments the small molecule binds to specific sequences of the human FGFR, for example, residues R577, D519, C488, F489, S518, T521, E522, D554, G555, P556, Q574, P587, P579, W691, T695, P702, G703 or P705 of FGFR1. In a preferred embodiment, a small molecule of the invention may bind to one or more residues in the FGF receptor which make up the small cavities or pockets of the asymmetric contact interface. For example, a small molecule of the invention may bind to one or more of the following residues in the β1-β2 loop of one monomer of the RTK, the β3-αC loop of one monomer of the RTK, the β4-B5 loop of one monomer of the RTK, the αD-αE loop of one monomer of the RTK, the αF helix of one monomer of the RTK and the αF-αG loop of one monomer of the RTK.

Thus, in some embodiments, a small molecule of the invention may bind to contiguous or non-contiguous amino acid residues and function as a molecular wedge that prevents the motion required for trans autophosphorylation of the RTK that enables tyrosine kinase activation. A small molecule of the invention may also act to prevent homotypic RTK interactions or destabilize the asymmetric contact interface. One of skill in the art will appreciate that, in some embodiments, a small molecule of the invention may be easily targeted to the corresponding residues in other type IV RTKs, e.g., those residues that form similar pockets or cavities or those in the same position by structural alignment or sequence alignment.

In a specific embodiment, a small molecule of the invention binds to a conformational epitope or a discontinuous epitope on a type IV RTK. The conformational or discontinuous epitope may be composed of two or more residues from the asymmetric contact interface from a type IV RTK, e.g., a human FGFR. For example, the conformational or discontinuous epitope may be composed of two or more of the residues R577, D519, C488, F489, S518, T521, E522, D554, G555, P556, Q574, P587, P579, W691, T695, P702, G703 and P705 of FGFR1. In a particular embodiment, a small molecule of the invention binds to a conformational epitope composed of 2 or more amino acids in a region of the RTK selected from the group consisting of the β1-β2 loop of one monomer of the RTK, the β3-αC loop of one monomer of the RTK, the β4-B5 loop of one monomer of the RTK, the αD-αE loop of one monomer of the RTK, the αF helix of one monomer of the RTK and the αF-αG loop of one monomer of the RTK. As indicated above, the small molecules of the invention may bind to all of the amino acid residues forming a pocket or a cavity identified in an asymmetric contact interface of an RTK or they may bind to a subset of the residues forming the pocket or the cavity. It is to be understood that, in certain embodiments, when reference is made to a small molecule of the invention binding to an epitope, e.g., a conformational epitope, the intention is for the small molecule to bind only to those specific residues that make up the epitope (e.g., the pocket or cavity of the asymmetric contact interface) and not other residues in the linear amino acid sequence of the receptor.

In another embodiment, a small molecule of the invention binds to amino acid residues Arg579 or Arg580 of human FGFR2, or the corresponding residues in FGFR3 or FGFR4. The residues Arg579 or Arg580 of human FGFR2 are analogous to the residue Arg576 of FGFR1 and are part of the FGFR2 asymmetric contact interface. In another embodiment, the small molecule binds to an amino acid residue selected from the group consisting of C491, F492, N662, G663, R664, L665, P666, V667, K668, W669, R577, R579, R580, P581, P582, E585, Y589, 5587, Y588, D589, I590, P705, G706, P708, F713, K724, A726, N727, C728, T729, N730 and E731 of FGFR2. The structure based sequence alignments presented in FIGS. 9 and 12 depict the residues involved in asymmetric contact formation; these residues are conserved in FGFR1, FGFR2, FGFR3 and FGFR4. Accordingly, in one embodiment of the invention, the small molecule binds to equivalent residues in FGFR3 or FGFR4. Small molecules of the invention may exert their inhibitory effect on receptor activation by preventing critical homotypic interactions (such as salt bridges) between asymmetric contact interfaces of type-IV RTKs that are essential for trans autophosphorylation. Small molecules of the invention may allow dimerization of the RTK, e.g., FGFR, while preventing trans autophosphorylation. Structure based sequence alignment has shown the conservation of residues involved in the formation of asymmetric contact interfaces found in structures of both active FGFR1 and FGFR2 (FIG. 9A). Thus in some embodiments, small molecules of the invention may be targeted to the conserved regions of the asymmetric contact interfaces of type IV RTKs.

In preferred embodiments, a small molecule of the invention binds to an asymmetric contact interface of an RTK, e.g., an FGFR, with high affinity, for example, with an affinity of a K_{D} of 1×10⁻⁷ M or less, a K_{D} of 5×10⁻⁸ M or less, a K_{D} of 1×10⁻⁸ M or less, a K_{D} of 5×10⁻⁹ M or less, or a K_{D} of between 1×10⁻⁸M and 1×10⁻¹⁰ M or less.

Small molecule inhibitors of the invention may be made or selected by several methods known in the art. Screening procedures can be used to identify small molecules from libraries which bind desired asymmetric contact interfaces of a RTK. One method, Chemetics® (Nuevolutions) uses DNA tags for each molecule in the library to facilitate selection. The Chemetics® system allows screening of millions of compounds for target binding. Patents related to small molecule libraries and tag based screening are U.S. Pat. Application Nos. 20070026397; 20060292603; 20060269920; 20060246450; 20060234231; 20060099592; 20040049008; 20030143561 which are incorporated herein by reference in their entirety.

Other well known methods that may be used to identify small molecules from libraries which bind desired asymmetric contact interfaces of a RTK, e.g., an FGFR, include methods which utilize libraries in which the library members are tagged with an identifying label, that is, each label present in the library is associated with a discreet compound structure present in the library, such that identification of the label tells the structure of the tagged molecule. One approach to tagged libraries utilizes oligonucleotide tags, as described, for example, in PCT Publication No. WO 2005/058479 A2 (the Direct Select™ technology) and in U.S. Pat. Nos. 5,573,905; 5,708,153; 5,723,598, 6,060,596 published PCT applications WO 93/06121; WO 93/20242; WO 94/13623; WO 00/23458; WO 02/074929 and WO 02/103008, and by Brenner and Lerner (Proc. Natl. Acad. Sci. USA 89, 5381-5383 (1992); Nielsen and Janda (Methods: A Companion to Methods in Enzymology 6, 361-371 (1994); and Nielsen, Brenner and Janda (J. Am. Chem. Soc. 115, 9812-9813 (1993)), the entire contents of each of which are incorporated herein by reference in their entirety. Such tags can be amplified, using for example, polymerase chain reaction, to produce many copies of the tag and identify the tag by sequencing. The sequence of the tag then identifies the structure of the binding molecule, which can be synthesized in pure form and tested for activity.

Preparation and screening of combinatorial chemical libraries is well known to those skilled in the art. Such combinatorial chemical libraries which may be used to identify moieties of the invention include, but are not limited to, peptide libraries (see, e.g., U.S. Pat. No. 5,010,175, Furka, Int. J. Pept. Prot. Res. 37:487 493 (1991) and Houghton et al., Nature 354:84 88 (1991)). Other chemistries for generating chemical diversity libraries are well known in the art and can be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909 6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217 9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Russell & Sambrook, all supra), peptide nucleic acid libraries (see, e.g., U.S. Pat. No. 5,539,083), carbohydrate libraries (see, e.g., Liang et al., Science, 274:1520 1522 (1996) and U.S. Pat. No. 5,593,853), small organic molecule libraries (see, e.g., benzodiazepines, Baum C&E N, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like). Each of the foregoing publications is incorporated herein by reference. Public databases are also available and are commonly used for small molecule screening, e.g., PubChem (pubchem.ncbi.nlm.nih.gov), Zinc (Irwin and Shoichet (2005) J. Chem. Inf. Model. 45(1):177-82), and ChemBank (Seiler et al. (2008) Nucleic Acids Res. 36 (Database issue): D351-D359).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.). Moreover, since screening methodologies are so well defined, it is common to contract specialist firms to identify particular compounds for a target of interest (e.g., BioFocus DPI (biofocus.com), and Quantum Lead (q-lead.com)).

Other methods of selecting small molecules which are well known in the art, and may be applied to the methods of the present invention are Huang and Stuart L. Schreiber (1997) Proc Natl Acad Sci USA. 94(25): 13396-13401; Hung et al. (2005) Science 310:670-674; Zhang et al. (2007) Proc Natl Acad Sci 104: 4606-4611; or any of the methods reviewed in Gordon (2007) ACS Chem. Biol. 2:9-16, all of which are incorporated herein by reference in their entirety.

In addition to experimental screening methods, small molecules of the invention may be selected using virtual screening methods. Virtual screening technologies predict which small molecules from a library will bind to a protein, or a specific epitope therein, using statistical analysis and protein docking simulations. Most commonly, virtual screening methods compare the three-dimensional structure of a protein to those of small molecules in a library. Different strategies for modeling protein-molecule interactions are used, although it is common to employ algorithms which simulate binding energies between atoms, including hydrogen bonds, electrostatic forces, and van-der walls interactions. Typically, virtual screening methods can scan libraries of more than a million compounds and return a short list of small molecules which are likely to be strong binders. Several reviews of virtual screening methods are available, detailing the techniques which may be used to identify small molecules of the present invention (Engel et al. (2008) J. Am. Chem. Soc., 130 (15), 5115-5123; McInnes (2007). Curr Opin Chem. Biol. October; 11(5):494-502; Reddy et al. (2007) Curr Protein Pept Sci. August; 8(4):329-51; Muegge and Oloff (2006) Drug Discovery Today, 3(4): 405-411; Kitchen et al. (2004) Nature Reviews Drug Discovery 3, 935-949). Further examples of small molecule screening can be found in U.S. 2005/0124678, which is incorporated herein by reference.

Small molecules of the invention may contain one of the scaffold structures depicted in the table below. The references cited in the table are incorporated herein by reference in their entirety. The groups R₁, R₂, R₃ and R₄ are limited only in that they should not interfere with, or significantly inhibit, the indicated reaction, and can include hydrogen, alkyl, substituted alkyl, heteroalkyl, substituted heteroalkyl, cycloalkyl, heterocycloalkyl, substituted cycloalkyl, substituted heterocycloalkyl, aryl, substituted aryl, arylalkyl, heteroarylalkyl, substituted arylalkyl, substituted heteroarylalkyl, heteroaryl, substituted heteroaryl, halogen, alkoxy, aryloxy, amino, substituted amino and others as are known in the art. Suitable substituents include, but are not limited to, alkyl, alkoxy, thioalkoxy, nitro, hydroxyl, sulfhydryl, aryloxy, aryl-S—, halogen, carboxy, amino, alkylamino, dialkylamino, arylamino, cyano, cyanate, nitrile, isocyanate, thiocyanate, carbamyl, and substituted carbamyl.

TABLE 1

| | | Aldehyde/ | Carboxylic | | |
|---|---|---|---|---|---|
| Scaffolds | Amine | Ketone | acid | Other | Reference |
| 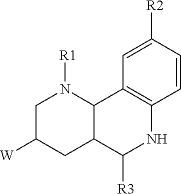 | 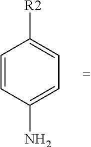 | 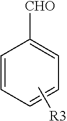 | | 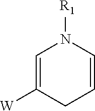 | Carranco, I., et al. (2005) J. Comb. Chem. 7:33-41 |
| 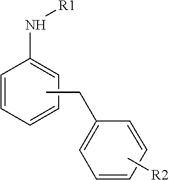 | amines | benzaldehydes and furfural | | 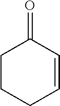 | Rosamilin, A. E., et al. (2005) Organic Letters 7:1525- 1528 |
| 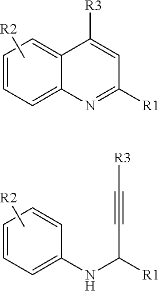 | 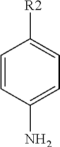 | 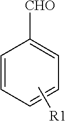 | | 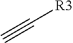 | Syeda Huma, H. Z. et al. (2002) Tet Lett 43:6485- 6488 |
| 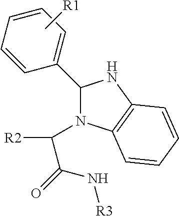 | 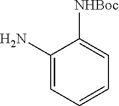 | R2—CHO | 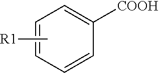 | ≡N—R3 | Tempest, P., et al. (2001) Tet Lett 42:4959- 4962 |
| 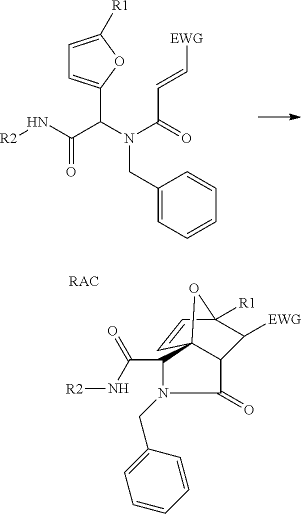 | 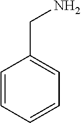 | 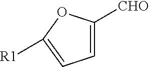 | 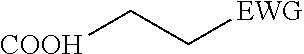 | 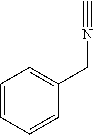 | Paulvannan, K. (1999) Tet Lett 40:1851- 1854 |
| 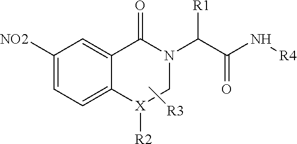 | | R1—CHO | 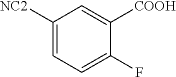 | ≡N—R4 | Tempest, P., et al. (2001) Tet Lett 42:4963- 4968 |
| 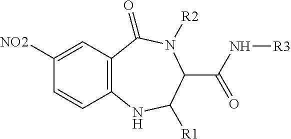 | R2—NH₂ | 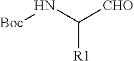 | 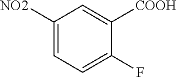 | ≡N—R3 | Tempest, P., et al. (2003) Tet Lett 44:1947- 1950 |
| 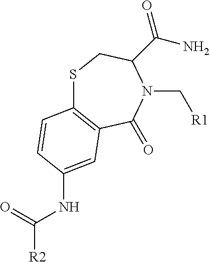 | | R1—CHO | R2—COOH | 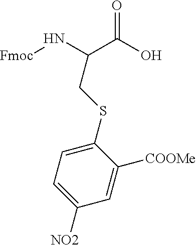 | Nefzi, A., et al. (1999) Tet Lett 40:4939- 4942 |
| 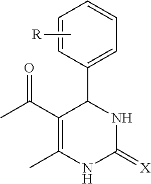 | | 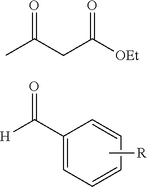 | | 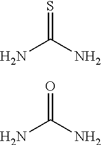 | Bose, A. K., et al. (2005) Tet Lett 46:1901- 1903 |
| 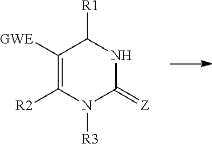 | 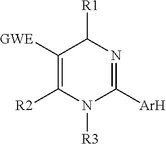 | 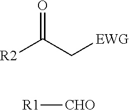 | | 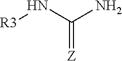 | Stadler, A. and Kappe, C. O. (2001) J. Comb. Chem. 3:624- 630; Lengar, A. and Kappe, C. O. (2004) Organic Letters 6:771-774 |
| 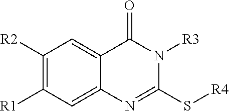 | wide range of primary aliphatic amines | | 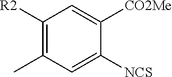 | 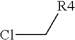 | Ivachtchenko, A. V., et al. (2003) J. Comb. Chem. 5:775-788 |
| 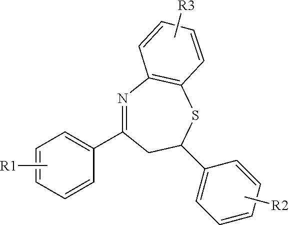 | | 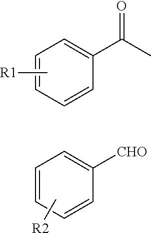 | | 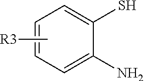 | Micheli, F., et al. (2001) J. Comb. Chem. 3:224-228 |
| 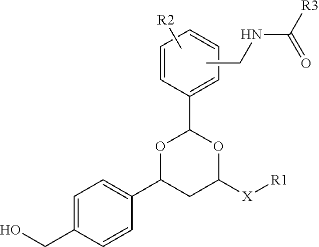 | R1—HS R1—NH₂ | 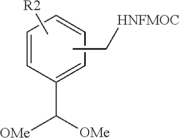 | 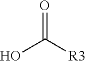 | 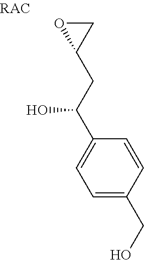 | Sternson, S. M., et al. (2001) Org. Lett. 3:4239- 4242 |
| 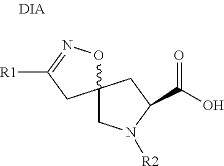 | | 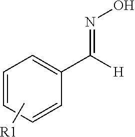 | | 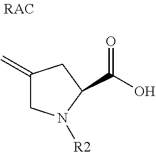 | Cheng, W. -C., et al. (2002) J. Org. Chem. 67:5673- 5677; Park, K. -H., et al. (2001) J Comb Chem 3:171-176 |
| 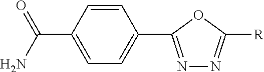 | 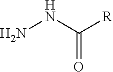 | | | 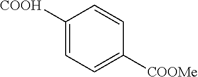 | Brown, B. J. et al. (2000) Synlett 1:131-133 |
| 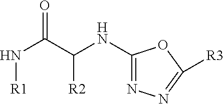 | R1—NH₂ | | 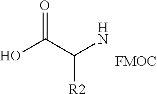 | 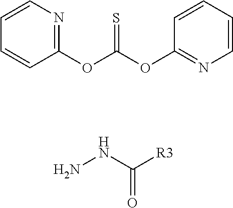 | Kilburn, J. P., et al. (2001) Tet Lett 42:2583- 2586 |
| 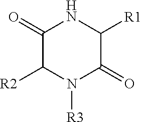 | amino acid | | amino acid ester | | del Fresno, M., et al. (1998) Tet Lett 39:2639- 2642 |
| 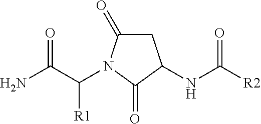 | amino acid | | carboxylic acids | | Alvarez- Gutierrez, J. M. et al. (2000) Tet Lett 41:609- 612 |
| 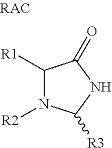 | | R2—CHO | 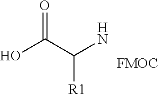 | 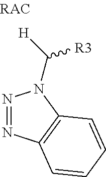 | Rinnová, M., et al. (2002) J. Comb. Chem 4:209- 213 |
| 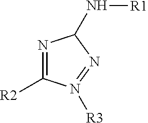 | R1—NH₂ | | 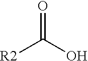 | 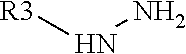 | Makara, G. M., et al. (2002) Organic Lett 4:1751- 1754 |
| 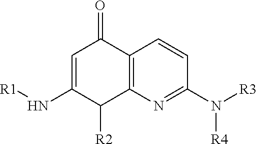 | | | | 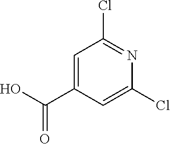 | Schell, P., et al. (2005) J. Comb. Chem 7:96-98 |
| 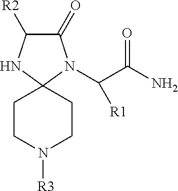 | | | amino acids | | Feliu L., et al. (2003) J. Comb. Chem. 5:356-361 |
| 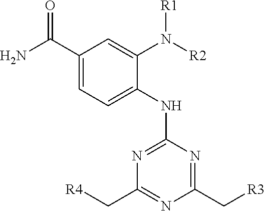 | Amines | Aldehydes | | | |
| 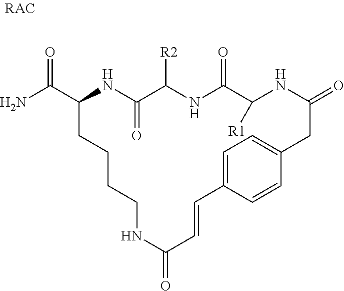 | | | amino acids | | Hiroshige, M., et al. (1995) J. Am. Chem. Soc. 117: 11590- 11591 |
| 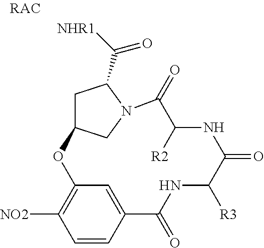 | | | amino acids | | Bose, A. K., et al. (2005) Tet Lett 46:1901- 1903 |

II. Peptidic Molecules which Bind an Asymmetric Contact Interface of a Human Receptor Tyrosine Kinase

In another aspect of the invention, the moiety that binds to an asymmetric contact interface of a human receptor tyrosine kinase is a peptidic molecule. The peptidic molecules may be designed based on an asymmetric contact interface of a RTK or a consensus sequence derived from such a domain.

In one embodiment, the peptidic moieties of the invention may comprise an entire protein domain, for example, a domain which comprises the entire asymmetric contact interface of FGFR1. Such a peptidic molecule binds the RTK and acts as an antagonist by preventing trans autophosphorylation and activation of the RTK. In some embodiments, the peptidic moieties of the invention may have as little as 50% identity to a domain of a RTK, such as a Type IV RTK, e.g., a peptidic moiety of the invention may be at least 50% identical, at least 60% identical, at least 70% identical, at least 80% identical, at least 90% identical, or at least 95%, 96%, 97%, 98% or 99% identical to an asymmetric contact interface domain, or a portion thereof, of a RTK. In a specific embodiment, the peptidic moiety of the invention is at least 80% identical, at least 90% identical, or at least 95%, 96%, 97%, 98% or 99% identical to amino acid residues 576-594 of human FGFR1 or 579-597 of human FGFR1.

In some embodiments, the peptidic moiety of the invention binds to or comprises specific sequences of a human FGFR, for example, residues R577, D519, C488, F489, S518, T521, E522, D554, G555, P556, Q574, P587, P579, W691, T695, P702, G703 and P705 of FGFR1. In other embodiments, the peptidic moiety of the invention binds to or comprises specific residues of regions of human FGFR1, for example, the β1-β2 loop of one monomer of FGFR1, the β3-αC loop of one monomer of FGFR1, the β4-B5 loop of one monomer of FGFR1, the αD-αE loop of one monomer of FGFR1, the αF helix of one monomer of FGFR1 and the αF-αG loop of one monomer of FGFR1.

In a preferred embodiment, a peptidic moiety of the invention may bind to (or comprise or consist of) one or more residues in a FGFR which make up the small cavities or pockets of the asymmetric contact interface. For example, a peptidic molecule of the invention may bind to (or comprise or consist of) one or more of the following residues in the asymmetric contact interface of the human FGFR1: R577, D519, C488, F489, S518, T521, E522, D554, G555, P556, Q574, P587, P579, W691, T695, P702, G703 and P705 of FGFR1.

A peptidic moiety of the invention may bind to contiguous or non-contiguous amino acid residues and function as a molecular wedge that prevents the ligand-induced trans autophosphorylation required for positioning of the asymmetric contact interface of the RTK at a distance and orientation that enables tyrosine kinase activation. In some embodiments, the moiety does not involve the nucleotide binding site of the catalytic domain of the RTK. In other embodiments, the peptidic molecule of the invention may act to prevent homotypic receptor interactions or destabilize the ligand-receptor interaction site. In some preferred embodiments, a peptidic molecule of the invention may bind to (or comprise or consist of) one or more of the following residues on the human FGFR1: R577, D519, C488, F489, S518, T521, E522, D554, G555, P556, Q574, P587, P579, W691, T695, P702, G703 or P705. The peptidic moieties of the invention may bind to (or comprise or consist of) all of the amino acid residues forming a pocket or a cavity of an asymmetric contact interface or they may bind to (or comprise or consist of) a subset of the residues forming the pocket or the cavity. One of skill in the art will appreciate that, in some embodiments, a peptidic molecule of the invention may be easily targeted to the corresponding residues in other type IV RTKs, e.g., those residues that form similar pockets or cavities or those in the same position by structural alignment or sequence alignment.

In a specific embodiment, a peptidic molecule of the invention binds to a conformational epitope or a discontinuous epitope on a type IV RTK. The conformational or discontinuous epitope may be composed of two or more residues from the asymmetric contact interface regions from a type IV RTK, e.g., a human FGFR, e.g., a human FGFR1, FGFR2, FGFR3 or FGFR4 receptor. For example, the conformational or discontinuous epitope may be composed of two or more of the residues selected from the group consisting of R577, D519, C488, F489, S518, T521, E522, D554, G555, P556, Q574, P587, P579, W691, T695, P702, G703 and P705 of FGFR1. In a particular embodiment, a peptidic molecule of the invention binds to a conformational epitope composed of 2 or more amino acids of a region of the RTK selected from the group consisting of the β1-β2 loop of one monomer of the RTK, the β3-αC loop of one monomer of the RTK, the β4-B5 loop of one monomer of the RTK, the αD-αE loop of one monomer of the RTK, the αF helix of one monomer of the RTK and the αF-αG loop of one monomer of the RTK.

In another embodiment, a peptidic moiety of the invention binds to a contiguous epitope on the RTK, e.g., a FGFR, e.g., FGFR1, FGFR2, FGFR3 or FGFR4. In one embodiment, the contiguous epitope is composed of two or more residues in the asymmetric contact interface of the fibroblast growth factor receptor. In another embodiment, the contiguous epitope is an epitope selected from the group consisting of the β1-β2 loop of a monomer of the RTK, the β3-αC loop of a monomer of the RTK, the β4-B5 loop of a monomer of the RTK, the αD-αE loop of a monomer of the RTK, the αF helix of a monomer of the RTK and the αF-αG loop of a monomer of the RTK.

In another embodiment, a peptidic molecule of the invention binds to amino acid residues Arg579 or Arg580 of human FGFR2 or the corresponding residues in FGFR3 or FGFR4. The residues Arg579 or Arg580 of human FGFR2 are analogous to the residue Arg576 of FGFR1 and are part of the FGFR2 asymmetric contact interface. In another embodiment, a peptidic molecule of the invention binds to an amino acid residue selected from the group consisting of C491, F492, N662, G663, R664, L665, P666, V667, K668, W669, R577, R579, R580, P581, P582, E585, Y589, 5587, Y588, D589, I590, P705, G706, P708, F713, K724, A726, N727, C728, T729, N730 and E731 of FGFR2. In yet another embodiment, a peptidic molecule of the invention binds to an equivalent amino acid residue in FGFR3 or FGFR4. Peptidic molecules of the invention may exert their inhibitory effect on receptor activation by preventing critical homotypic interactions (such as salt bridges) between asymmetric contact interfaces of type-IV RTKs that are essential for positioning the kinase dimers at a distance and orientation essential for ligand-induced trans autophosphorlyation and tyrosine kinase activation. Peptidic molecules of the invention may allow dimerization of the RTK, e.g., FGFR, while preventing trans autophosphorylation. Structure based sequence alignment has shown the conservation of residues involved in the formation of asymmetric contact interfaces found in structures of both active FGFR1 and FGFR2 (FIG. 9A). Thus in some embodiments, peptidic molecules of the invention may be targeted to the conserved regions of the asymmetric contact interfaces of type IV RTKs (see also, FIG. 12).

The peptidic moieties of the invention may be peptides comprising or consisting of any of the amino acid sequences identified herein, such as R577, D519, C488, F489, S518, T521, E522, D554, G555, P556, Q574, P587, P579, W691, T695, P702, G703 and P705 of FGFR1. For example, peptidic moieties of the invention may be peptides comprising or consisting of any of the following regions of the RTK: the β1-β2 loop of one monomer of the RTK, the β3-αC loop of one monomer of the RTK, the β4-B5 loop of one monomer of the RTK, the αD-αE loop of one monomer of the RTK, the αF helix of one monomer of the RTK and the αF-αG loop of one monomer of the RTK.

A peptide molecule of the invention may be further modified to increase its stability, bioavailability or solubility. For example, one or more L-amino acid residues within the peptidic molecules may be replaced with a D-amino acid residue. The term “mimetic” as applied to the peptidic molecules of the present invention is intended to include molecules which mimic the chemical structure of a D-peptidic structure and retain the functional properties of the D-peptidic structure. The term “mimetic” is further intended to encompass an “analogue” and/or “derivative” of a peptide as described below. Approaches to designing peptide analogs, derivatives and mimetics are known in the art. For example, see Farmer, P. S. in Drug Design (E. J. Ariens, ed.) Academic Press, New York, 1980, vol. 10, pp. 119-143; Ball. J. B. and Alewood, P. F. (1990) J. Mol. Recognition. 3:55; Morgan, B. A. and Gainor, J. A. (1989) Ann. Rep. Med. Chem. 24:243; and Freidinger, R. M. (1989) Trends Pharmacol. Sci. 10:270. See also Sawyer, T. K. (1995) “Peptidomimetic Design and Chemical Approaches to Peptide Metabolism” in Taylor, M. D. and Amidon, G. L. (eds.) Peptide-Based Drug Design: Controlling Transport and Metabolism, Chapter 17; Smith, A. B. 3rd, et al. (1995) J. Am. Chem. Soc. 117:11113-11123; Smith, A. B. 3rd, et al. (1994) J. Am. Chem. Soc. 116:9947-9962; and Hirschman, R., et al. (1993) J. Am. Chem. Soc. 115:12550-12568.

As used herein, a “derivative” of a peptidic molecule of the invention refers to a form of the peptidic molecule in which one or more reaction groups on the molecule have been derivatized with a substituent group. Examples of peptide derivatives include peptides in which an amino acid side chain, the peptide backbone, or the amino- or carboxy-terminus has been derivatized (e.g., peptidic compounds with methylated amide linkages). As used herein an “analogue” of a peptidic molecule of the invention to a peptidic molecule which retains chemical structures of the molecule necessary for functional activity of the molecule yet which also contains certain chemical structures which differ from the molecule. An example of an analogue of a naturally-occurring peptide is a peptide which includes one or more non-naturally-occurring amino acids. As used herein, a “mimetic” of a peptidic molecule of the invention refers to a peptidic molecule in which chemical structures of the molecule necessary for functional activity of the molecule have been replaced with other chemical structures which mimic the conformation of the molecule. Examples of peptidomimetics include peptidic compounds in which the peptide backbone is substituted with one or more benzodiazepine molecules (see e.g., James, G. L. et al. (1993) Science 260:1937-1942).

Analogues of the peptidic molecules of the invention are intended to include molecules in which one or more L- or D-amino acids of the peptidic structure are substituted with a homologous amino acid such that the properties of the molecule are maintained. Preferably conservative amino acid substitutions are made at one or more amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Non-limiting examples of homologous substitutions that can be made in the structures of the peptidic molecules of the invention include substitution of D-phenylalanine with D-tyrosine, D-pyridylalanine or D-homophenylalanine, substitution of D-leucine with D-valine or other natural or non-natural amino acid having an aliphatic side chain and/or substitution of D-valine with D-leucine or other natural or non-natural amino acid having an aliphatic side chain.

The term mimetic, and in particular, peptidomimetic, is intended to include isosteres. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) well known to those skilled in the art. Such modifications include modifications of the amide nitrogen, the α-carbon, amide carbonyl, complete replacement of the amide bond, extensions, deletions or backbone crosslinks. Several peptide backbone modifications are known, including ψ[CH₂S], ψ[CH₂NH], ψ[CSNH₂], ψ[NHCO], ψ[COCH₂], and ψ[(E) or (Z) CH═CH]. In the nomenclature used above, iv indicates the absence of an amide bond. The structure that replaces the amide group is specified within the brackets.

Other possible modifications include an N-alkyl (or aryl) substitution (iv [CONR]), or backbone crosslinking to construct lactams and other cyclic structures. Other derivatives of the modulator compounds of the invention include C-terminal hydroxymethyl derivatives, O-modified derivatives (e.g., C-terminal hydroxymethyl benzyl ether), N-terminally modified derivatives including substituted amides such as alkylamides and hydrazides.

Peptidic molecules of the present invention may be made by standard methods known in the art. The peptidic molecule, e.g., asymmetric contact interface of an RTK, may be cloned from human cells using standard techniques, inserted in to a recombinant vector, and expressed in an in vitro cell system (e.g., by transfection of the vector into yeast cells). Alternatively, the peptidic molecules may be designed and synthesized de novo via known synthesis methods such as Atherton et al. (1989) Oxford, England: IRL Press. ISBN 0199630674; Stewart et al. (1984). 2nd edition, Rockford: Pierce Chemical Company, 91. ISBN 0935940030; Merrifield (1963) J. Am. Chem. Soc. 85: 2149-2154.

The peptidic molecules can then be tested for functional activity using any of the assays described herein, e.g., those described in the Examples section below.

III. Antibodies which Bind to an Asymmetric Contact Interface of a Human Receptor Tyrosine Kinase

In one aspect of the invention, the moiety that binds to an asymmetric contact interface of a human receptor tyrosine kinase is an antibody or an antigen binding fragment thereof that is able to enter a cell, such as an intrabody.

The term “antibody” as referred to herein, includes whole antibodies and any antigen binding fragment (i.e., “antigen-binding portion”) or single chains thereof. The term “antibody” includes intracellular antibodies such as intrabodies. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g., the asymmetric contact interface of an RTK). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3rd ed. 1993); (iv) a Fd fragment consisting of the V_{H} and C_{H}1 domains; (v) a Fv fragment consisting of the V_{L} and V_{H} domains of a single arm of an antibody, (vi) a dAb fragment (Ward et al., (1989) Nature 341:544-546), which consists of a V_{H} domain; (vii) an isolated complementarity determining region (CDR); and (viii) a nanobody, a heavy chain variable region containing a single variable domain and two constant domains. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds to an asymmetric contact interface of an RTK is substantially free of antibodies that specifically bind antigens other than the asymmetric contact interface of an RTK). Moreover, an isolated antibody may be substantially free of other cellular material and/or chemicals. An “isolated antibody” may, however, include polyclonal antibodies which all bind specifically to, e.g., an asymmetric contact interface of an RTK.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences. It will be appreciated by one of skill in the art that when a sequence is “derived” from a particular species, said sequence may be a protein sequence, such as when variable region amino acids are taken from a murine antibody, or said sequence may be a DNA sequence, such as when variable region encoding nucleic acids are taken from murine DNA. A humanized antibody may also be designed based on the known sequences of human and non-human (e.g., murine or rabbit) antibodies. The designed antibodies, potentially incorporating both human and non-human residues, may be chemically synthesized. The sequences may also be synthesized at the DNA level and expressed in vitro or in vivo to generate the humanized antibodies.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “antibody mimetic” or “antibody mimic” is intended to refer to molecules capable of mimicking an antibody's ability to bind an antigen, but which are not limited to native antibody structures. Examples of such antibody mimetics include, but are not limited to, Adnectins (i.e., fibronectin based binding molecules), Affibodies, DARPins, Anticalins, Avimers, and Versabodies all of which employ binding structures that, while they mimic traditional antibody binding, are generated from and function via distinct mechanisms. The embodiments of the instant invention, as they are directed to antibodies, or antigen binding portions thereof, also apply to the antibody mimetics described above.

As used herein, an antibody that “specifically binds” to an asymmetric contact interface of a RTK is intended to refer to an antibody that binds to an asymmetric contact interface domain of a RTK with a K_{D} of 1×10⁻⁷ M or less, more preferably 5×10⁻⁸ M or less, more preferably 1×10⁻⁸ M or less, more preferably 5×10⁻⁹ M or less

The term “does not substantially bind” to a protein or cells, as used herein, means does not bind or does not bind with a high affinity to the protein or cells, i.e. binds to the protein or cells with a K_{D} of 1×10⁻⁶ M or more, more preferably 1×10⁻⁵ M or more, more preferably 1×10⁻⁴ M or more, more preferably 1×10⁻³ M or more, even more preferably 1×10⁻² M or more.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore® system.

As used herein, the term “high affinity”, when referring an IgG type antibody, refers to an antibody having a K_{D} of 10⁻⁸ M or less, more preferably 10⁻⁹ M or less and even more preferably 10⁻¹⁰ M or less for an asymmetric contact interface domain of a RTK. However, “high affinity” binding can vary for other antibody isotypes. For example, “high affinity” binding for an IgM isotype refers to an antibody having a K_{D} of 10⁻⁷ M or less, more preferably 10⁻⁸ M or less, even more preferably 10⁻⁹ M or less.

### Antibodies

The antibodies of the invention bind specifically to an asymmetric contact interface domain of a RTK, e.g., member of the human type IV family of receptor tyrosine kinases. In preferred embodiments, the antibodies, or antigen binding portions thereof, of the invention bind to an asymmetric contact interface of an RTK and, thus, inhibit ligand-induced trans autophosphorylation and downstream signaling by the receptor.

The antibodies of the invention are selected or designed to bind to specific asymmetric contact interfaces of the RTK, for example, an FGFR, for example, FGFR1, FGFR2, FGFR3 or FGFR4. In other embodiments the antibodies, or antigen binding portions thereof, are selected or designed to bind proteins sharing homology to a domain of the RTK, e.g., a human FGFR. For example, an antibody may be selected or designed to bind a domain which is at least 50% identical, at least 60% identical, at least 70% identical, at least 80% identical, at least 90% identical, or at least 95%, 96%, 97%, 98% or 99% identical to an asymmetric contact interface of the FGFR, e.g., FGFR1. Such an antibody, or antigen binding portion thereof, would be able to bind protein domains, possibly in other RTKs, which are functionally similar to the asymmetric contact interface of FGFR1.

The antibodies, or antigen binding portions thereof, of the present invention may also be selected or designed to bind a particular motif or consensus sequence derived from an asymmetric contact interface of a RTK, e.g., a human type IV RTK, allowing the antibodies, or antigen binding portions thereof, to specifically bind asymmetric contact interface epitopes or domains which are shared among members of the RTK family. Such a linear consensus sequence may be found, for example, by using a sequence alignment algorithm to align domains of various RTKs, e.g., asymmetric contact interfaces across RTK types or across species. One of skill in the art may align the protein sequences of, for example, the FGFR1 asymmetric contact interfaces from various species (e.g., human, mouse, rat) to determine which protein residues are conserved in at least 40%, at least 50%, at least 60%, at least 70%, at least 80%, at least 90%, or at least 100% of the sequences being aligned. Such a consensus sequence may then be used to generate antibodies or other moieties which specifically bind the consensus sequence and, thus, will bind the most conserved residues of the RTK. One of skill in the art should appreciate that the most highly conserved residues are those which have been preserved through evolution and are most likely to be important for protein function. Alternatively, if the alignment is made across various classes of RTKs, antibodies generated toward such consensus sequences would allow the antibodies to bind a similar asymmetric contact interface in multiple RTK types.

The antibodies of the present invention do not bind to the nucleotide binding site of the catalytic domain of the RTK, e.g., the FGFR. Therefore, the antibodies described herein do not antagonize the ability of the receptor to bind its target ligand.

In some embodiments the antibody or antigen binding portion thereof binds to specific sequences of the human FGFR1, for example, residues R577, D519, C488,

F489, S518, T521, E522, D554, G555, P556, Q574, P587, P579, W691, T695, P702, G703 and P705 of FGFR1.

In other embodiments, the antibodies, or antigen binding portions thereof, bind protein motifs or consensus sequences which represent a three dimensional structure in the protein. Such motifs or consensus sequences would not represent a contiguous string of amino acids, but a non-contiguous amino acid arrangement that results from the three-dimensional folding of the RTK (i.e., a “structural motif” or “non-linear epitope”). An example of such a motif would be the asymmetric contact interface of a RTK.

In a preferred embodiment, an antibody or antigen binding portion thereof of the invention may bind to one or more residues in the RTK which make up the small cavities or pockets of an asymmetric contact interface of an RTK. For example, an antibody or antigen binding portion thereof of the invention may bind to one or more of the following residues in the asymmetric contact interface of FGFR1: R577, D519, C488, F489, S518, T521, E522, D554, G555, P556, Q574, P587, P579, W691, T695, P702, G703 and P705 of FGFR1. In another embodiment, the antibody or antigen binding portion thereof of the invention may bind of one or more residues in the asymmetric contact interface of FGFR2: C491, F492, N662, G663, R664, L665, P666, V667, K668, W669, R577, R579, R580, P581, P582, E585, Y589, 5587, Y588, D589, I590, P705, G706, P708, F713, K724, A726, N727, C728, T729, N730 and E731 of FGFR2. In yet another embodiment, the antibody or antigen-binding portion thereof may bind to one or more equivalent residues in FGFR3 or FGFR4.

Thus, in some embodiments, an antibody or antigen binding portion thereof of the invention may bind to contiguous or non-contiguous amino acid residues and function as a molecular wedge that prevents the ligand-induced trans autophosphorylation required for positioning of the asymmetric contact interface of the RTK at a distance and orientation that enables tyrosine kinase activation. In some embodiments, an antibody or antigen binding portion thereof of the invention may also act to prevent homotypic receptor interactions or destabilize the ligand-receptor interaction site.

One of skill in the art will appreciate that, in some embodiments, an antibody or antigen binding portion thereof of the invention may be easily targeted to the corresponding asymmetric contact interface residues in other type IV RTKs, e.g., those residues that form similar pockets or cavities or those in the same position by structural alignment or sequence alignment.

In a specific embodiment, an antibody or antigen binding portion thereof of the invention binds to a conformational epitope or a discontinuous epitope on a type IV RTK. The conformational or discontinuous epitope may be composed of two or more residues from the asymmetric contact interface of an RTK, e.g., the human FGFR1, FGFR2, FGFR3 or FGFR4. For example, the conformational or discontinuous epitope may be composed of two or more of the residues selected from the group consisting of R577, D519, C488, F489, S518, T521, E522, D554, G555, P556, Q574, P587, P579, W691, T695, P702, G703 and P705 of FGFR1.

As indicated above, the antibodies of the invention may bind to all of the amino acid residues forming a pocket or a cavity of an asymmetric contact interface or they may bind to a subset of the residues forming the pocket or the cavity. It is to be understood that, in certain embodiments, when reference is made to an antibody of the invention binding to an epitope, e.g., a conformational epitope, the intention is for the antibody to bind only to those specific residues that make up the epitope (e.g., the pocket or cavity of the asymmetric contact interface) and not other residues in the linear amino acid sequence of the receptor.

In another embodiment, the antibody or antigen-binding fragment of the invention binds to amino acid residues Arg579 or Arg580 of human FGFR2, or the corresponding residues in FGFR3 or FGFR4. The residues Arg579 or Arg580 of human FGFR2 are analogous to the residue Arg576 of FGFR1 and are part of the FGFR2 asymmetric contact interface. In another embodiment, the antibody or antigen-binding fragment of the invention binds to an amino acid residue selected from the group consisting of C491, F492, N662, G663, R664, L665, P666, V667, K668, W669, R577, R579, R580, P581, P582, E585, Y589, S587, Y588, D589, I590, P705, G706, P708, F713, K724, A726, N727, C728, T729, N730 and E731 of FGFR2. In another embodiment, the antibody or antigen-binding fragment of the invention binds to an equivalent residue in FGFR3 or FGFR4. Antibodies or antigen-binding antibody fragments of the invention may exert their inhibitory effect on receptor activation by preventing critical homotypic interactions (such as salt bridges) between asymmetric contact interfaces of type-IV RTKs that are essential for positioning the kinase dimers at a distance and orientation essential for ligand-induced trans autophosphorlyation and tyrosine kinase activation. Experiments discussed herein demonstrate that the asymmetric contact interface mediates RTK dimer formation and that dimerization is necessary but not sufficient for receptor trans autophosphorylation. Thus, antibodies or antigen-binding antibody fragments of the invention may allow dimerization of the RTK, e.g., FGFR, while preventing trans autophosphorylation. Structure based sequence alignment has shown the conservation of residues involved in the formation of asymmetric contact interfaces found in structures of both active FGFR1 and FGFR2 (FIG. 9A). Thus in some embodiments, antibodies or antigen-binding antibody fragments of the invention may be targeted to the conserved regions of the asymmetric contact interfaces of type IV RTKs.

In some embodiments, the antibody or antigen-binding portion thereof, binds to specific regions of a human fibroblast growth factor receptor, for example, the β1-β2 loop of one monomer of the RTK, the β3-αC loop of one monomer of the RTK, the β4-B5 loop of one monomer of the RTK, the αD-αE loop of one monomer of the RTK, the αF helix of one monomer of the RTK and the αF-αG loop of one monomer of the RTK.

In another embodiment, the antibody or antigen-binding portion thereof binds to a contiguous epitope on the RTK, e.g., FGFR. In one embodiment, the contiguous epitope is composed of two or more residues in an asymmetric contact interface of an FGFR. In another embodiment, the contiguous epitope is an epitope selected from the group consisting of the β1-β2 loop of a monomer of the RTK, the β3-αC loop of a monomer of the RTK, the β4-B5 loop of a monomer of the RTK, the αD-αE loop of a monomer of the RTK, the αF helix of a monomer of the RTK and the αF-αG loop of a monomer of the RTK.

In additional embodiments, antibodies or antigen-binding antibody fragments of the invention are selected or designed to bind specifically to a mutant asymmetric contact interface of a RTK. In preferred embodiments, the mutant RTK is a tumorigenic or an oncogenic mutant. In one specific embodiment, the antibody or antigen-binding antibody fragment is selected or designed to bind to an oncogenic FGFR mutant. RTK mutants which may be targeted by the antibodies or antigen-binding antibody fragments of the instant invention include, but are not limited to, fibroblast growth factor receptors with mutations in one or more of the following amino acids: R577, D519, C488, F489, S518, T521, E522, D554, G555, P556, Q574, P587, P579, W691, T695, P702, G703 and P705 of FGFR1. It should be appreciated by one of skill in the art that the methods of the invention would be applicable to other mutations in FGFRs or to mutations in other RTKs. One advantage of targeting a mutant RTK is that a therapeutic small molecule may bind to only the RTKs on cells containing the mutation, leaving healthy cells largely or entirely unaffected. Accordingly, in instances where the mutation is tumorigenic, only tumor cells would be targeted for therapy, potentially reducing side effects and dosage requirements.

Preferrably, the antibody binds to an asymmetric contact interface of a human RTK with a K_{D} of 5×10⁻⁸ M or less, a K_{D} of 1×10⁻⁸ M or less, a K_{D} of 5×10⁻⁹ M or less, or a K_{D} of between 1×10⁻⁸M and 1×10⁻¹⁰ M or less. Standard assays to evaluate the binding ability of the antibodies toward an asymmetric contact interface of a RTK, e.g., an FGFR, are known in the art, including for example, ELISAs, Western blots and RIAs. The binding kinetics (e.g., binding affinity) of the antibodies also can be assessed by standard assays known in the art, such as by ELISA, Scatchard and Biacore analysis.

### Intrabodies—Intracellular Antibodies

An antibody which can bind an intracellular epitope, e.g., an intrabody, is useful for binding to an asymmetric contact interface of an RTK and inhibiting ligand-induced trans autophosphorylation of the RTK. An intrabody comprises at least a portion of an antibody (e.g., an scFv) that is capable of specifically binding an antigen and which has been manipulated so that it can be expressed intracellularly and/or will bind an antigen intracellularly. Generally, an intrabody does not contain sequences coding for its secretion. When combined with methods for expression and/or targeting to precise intracellular locations inside mammalian cells, intrabodies are particularly useful for intracellular targets such as an asymmetric contact interface of an RTK. Generation of intrabodies is well-known to the skilled artisan and is described, for example, in U.S. Pat. Nos. 5,851,829; 5,965,371; 6,004,940; 6,072,036; and 5,965,371, the entire contents of each of which are expressly incorporated herein by reference. Furthermore, the construction of intrabodies is discussed in Ohage and Steipe, 1999, J. Mol. Biol. 291:1119-1128; Ohage et al., 1999, J. Mol. Biol. 291:1129-1134; and Wirtz and Steipe, 1999, Protein Science 8:2245-2250; and Stocks, M. R. Drug Disc. Today Vol 9, No. 22 Nov. 2004. Recombinant molecular biological techniques may also be used in the generation of intrabodies.

In one embodiment, a nucleic acid construct that expresses an intrabody can be transfected into target cells. An “antibody cassette” which encodes the intrabody may contain a sufficient number of nucleotides coding for the portion of an antibody capable of binding to the target RTK asymmetric contact interface operably linked to a promoter that will permit expression of the antibody in the cells of interest. The construct encoding the intrabody can be delivered to the target cell bind to the target RTK, thereby antagonizing the ligand-induced trans autophosphorylation of the RTK. In one preferred embodiment, the “intrabody gene” (antibody) of the antibody cassette utilizes a cDNA, encoding heavy chain variable (VH) and light chain variable (VL) domains of an antibody which can be connected at the DNA level by an appropriate oligonucleotide as a bridge of the two variable domains, which on translation, form a single peptide (referred to as a single chain variable fragment, “scFv”) capable of binding to a target such as an RTK protein. The intrabody gene preferably does not encode an operable secretory sequence and thus the expressed antibody remains within the cell.

In another embodiment, specific localization sequences can be attached to the intrabody polypeptide to direct the intrabody to a specific intracellular location. Intrabodies can be localized, for example, to the following intracellular locations: endoplasmic reticulum (Munro et al., 1987, Cell 48:899-907; Hangejorden et al., 1991, J. Biol. Chem. 266:6015); nucleus (Lanford et al., 1986, Cell 46:575; Stanton et al., 1986, PNAS 83:1772; Harlow et al., 1985, Mol. Cell. Biol. 5:1605; Pap et al., 2002, Exp. Cell Res. 265:288-93); nucleolar region (Seomi et al., 1990, J. Virology 64:1803; Kubota et al., 1989, Biochem. Biophys. Res. Comm. 162:963; Siomi et al., 1998, Cell 55:197); endosomal compartment (Bakke et al., 1990, Cell 63:707-716); mitochondrial matrix (Pugsley, A. P., 1989, “Protein Targeting”, Academic Press, Inc.); Golgi apparatus (Tang et al., 1992, J. Bio. Chem. 267:10122-6); liposomes (Letourneur et al., 1992, Cell 69:1183); peroxisome (Pap et al., 2002, Exp. Cell Res. 265:288-93); bans Golgi network (Pap et al., 2002, Exp. Cell Res. 265:288-93); and plasma membrane (Marehildon et al., 1984, PNAS 81:7679-82; Henderson et al., 1987, PNAS 89:339-43; Rhee et al., 1987, J. Virol. 61:1045-53; Schultz et al., 1984, J. Virol. 133:431-7; Otsuyama et al., 1985, Jpn. J. Can. Res. 76: 1132-5; Ratner et al., 1985, Nature 313:277-84).

The antibody cassette can be delivered to a target cell by any of the known means. One preferred delivery system is described in U.S. Pat. No. 6,004,940, the entire contents of which are incorporated herein by reference.

Anti-RTK antibodies suitable for use/expression as intrabodies in the methods of this invention can be readily produced by a variety of other methods. Such other methods include, but are not limited to, traditional methods of raising “whole” polyclonal antibodies, which can be modified to form single chain antibodies or screening of, e.g., phage display libraries to select for antibodies showing high specificity and/or avidity for an asymmetric contact interface of an RTK. Phase display library screening methods are described herein in some detail. In one embodiment, intrabodies of the invention retain at least about 75% of the binding effectiveness of the complete antibody (i.e., having the entire constant domain as well as the variable regions) to the antigen. In one embodiment, the intrabody retains at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the binding effectiveness of the complete antibody. In another embodiment, anti-RTK antibodies suitable for use as intrabodies can be produced by de novo production of diverse intracellular antibody libraries (see, e.g., Tanaka et al., 2003, Nucleic Acids Res., 31(5):e23)

In another embodiment, recombinantly expressed intrabody may be administered to a patient to mediate a prophylactic or therapeutic effect. To direct the intrabody intracellularly, an intrabody polypeptide can be associated with a “membrane permeable sequence”. Membrane permeable sequences are polypeptides capable of penetrating through the cell membrane from outside of the cell to the interior of the cell. When linked to another polypeptide, membrane permeable sequences can direct the translocation of that polypeptide across the cell membrane. Useful membrane permeable sequence include the hydrophobic region of a signal peptide (sec, e.g., Hawiger, 1999, Curr. Opin. Chem. Biol. 3:89-94; Hawiger, 1997, Curr. Opin. Immunol. 9:189-94; U.S. Pat. Nos. 5,807,746 and 6,043,339, the entire contents of which are incorporated herein by reference). The sequence of a membrane permeable sequence can be based on the hydrophobic region of any signal peptide. The signal peptides can be selected, e.g., from the SIGPEP database (see e.g., von; Heijne, 1987, Prot. Seq. Data Anal. 1:41-2; von Heijne and Abrahmsen, 1989, FEBS Lett.; 224:439-46). When a specific cell type is to be targeted for insertion of an intrabody polypeptide, the membrane permeable sequence is preferably based on a signal peptide endogenous to that cell type. In another embodiment, the membrane permeable sequence is a viral protein (e.g., Herpes Virus Protein VP22) or fragment thereof (see e.g., Phelan et al., 1998, Nat. Biotechnol. 16:440-3). A membrane permeable sequence with the appropriate properties for a particular intrabody and/or a particular target cell type can be determined empirically by assessing the ability of each membrane permeable sequence to direct the translocation of the intrabody across the cell membrane.

### Engineered and Modified Antibodies

The V_{H} and/or V_{L} sequences of an antibody prepared according the methods of the present invention and may be used as starting material to engineer a modified antibody, which modified antibody may have altered properties from the starting antibody. An antibody can be engineered by modifying one or more residues within one or both of the original variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter, and U.S. Pat. Nos. 5,530,101; 5,585,089; 5,693,762 and 6,180,370 to Queen et al.)

Framework sequences for antibodies can be obtained from public DNA databases or published references that include germline antibody gene sequences. For example, germline DNA sequences for human heavy and light chain variable region genes can be found in the “VBase” human germline sequence database (available on the Internet at mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{H} Segments Reveals a Strong Bias in their Usage” Eur. J. Immunol. 24:827-836; the contents of each of which are expressly incorporated herein by reference. As another example, the germline DNA sequences for human heavy and light chain variable region genes can be found in the Genbank database.

Antibody protein sequences are compared against a compiled protein sequence database using one of the sequence similarity searching methods called the Gapped BLAST (Altschul et al. (1997) Nucleic Acids Research 25:3389-3402), which is well known to those skilled in the art. BLAST is a heuristic algorithm in that a statistically significant alignment between the antibody sequence and the database sequence is likely to contain high-scoring segment pairs (HSP) of aligned words. Segment pairs whose scores cannot be improved by extension or trimming is called a hit. Briefly, the nucleotide sequences of VBASE origin (vbase.mrc-cpe.cam.ac.uk/vbase1/list2.php) are translated and the region between and including FR1 through FR3 framework region is retained. The database sequences have an average length of 98 residues. Duplicate sequences which are exact matches over the entire length of the protein are removed. A BLAST search for proteins using the program blastp with default, standard parameters except the low complexity filter, which is turned off, and the substitution matrix of BLOSUM62, filters for the top 5 hits yielding sequence matches. The nucleotide sequences are translated in all six frames and the frame with no stop codons in the matching segment of the database sequence is considered the potential hit. This is in turn confirmed using the BLAST program tblastx, which translates the antibody sequence in all six frames and compares those translations to the VBASE nucleotide sequences dynamically translated in all six frames. Other human germline sequence databases, such as that available from IMGT (http://imgt.cines.fr), can be searched similarly to VBASE as described above.

The identities are exact amino acid matches between the antibody sequence and the protein database over the entire length of the sequence. The positives (identities+substitution match) are not identical but amino acid substitutions guided by the BLOSUM62 substitution matrix. If the antibody sequence matches two of the database sequences with same identity, the hit with most positives would be decided to be the matching sequence hit.

Identified V_{H} CDR1, CDR2, and CDR3 sequences, and the V_{K} CDR1, CDR2, and CDR3 sequences, can be grafted onto framework regions that have the identical sequence as that found in the germline immunoglobulin gene from which the framework sequence derives, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Another type of variable region modification is to mutate amino acid residues within the V_{H} and/or V_{K} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays known in the art. For example, an antibody of the present invention may be mutated to create a library, which may then be screened for binding to an asymmetric contact interface of an RTK, e.g., a fibroblast growth factor receptor. Preferably conservative modifications (as discussed above) are introduced. The mutations may be amino acid substitutions, additions or deletions, but are preferably substitutions. Moreover, typically no more than one, two, three, four or five residues within a CDR region are altered.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. These strategies will be effective as long as the binding of the antibody to an asymmetric contact interface of the RTK is not compromised.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγR1, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII Additionally, the following combination mutants were shown to improve FcγRIII binding: T256A/S298A, S298A/E333A, S298A/K224A and S298A/E333A/K334A.

In still another embodiment, the C-terminal end of an antibody of the present invention is modified by the introduction of a cysteine residue as is described in U.S. Provisional Application Ser. No. 60/957,271, which is hereby incorporated by reference in its entirety. Such modifications include, but are not limited to, the replacement of an existing amino acid residue at or near the C-terminus of a full-length heavy chain sequence, as well as the introduction of a cysteine-containing extension to the c-terminus of a full-length heavy chain sequence. In preferred embodiments, the cysteine-containing extension comprises the sequence alanine-alanine-cysteine (from N-terminal to C-terminal).

In preferred embodiments the presence of such C-terminal cysteine modifications provide a location for conjugation of a partner molecule, such as a therapeutic agent or a marker molecule. In particular, the presence of a reactive thiol group, due to the C-terminal cysteine modification, can be used to conjugate a partner molecule employing the disulfide linkers described in detail below. Conjugation of the antibody to a partner molecule in this manner allows for increased control over the specific site of attachment. Furthermore, by introducing the site of attachment at or near the C-terminus, conjugation can be optimized such that it reduces or eliminates interference with the antibody's functional properties, and allows for simplified analysis and quality control of conjugate preparations.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 to Co et al. Additional approaches for altering glycosylation are described in further detail in U.S. Pat. No. 7,214,775 to Hanai et al., U.S. Pat. No. 6,737,056 to Presta, U.S. Pub No. 20070020260 to Presta, PCT Publication No. WO/2007/084926 to Dickey et al., PCT Publication No. WO/2006/089294 to Zhu et al., and PCT Publication No. WO/2007/055916 to Ravetch et al., each of which is hereby incorporated by reference in its entirety.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8^{-/-} cell lines were created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase alpha-L-fucosidase removes fucosyl residues from antibodies (Tarentino, A. L. et al. (1975) Biochem. 14:5516-23).

Additionally or alternatively, an antibody can be made that has an altered type of glycosylation, wherein that alteration relates to the level of sialyation of the antibody. Such alterations are described in PCT Publication No. WO/2007/084926 to Dickey et al., and PCT Publication No. WO/2007/055916 to Ravetch et al., both of which are incorporated by reference in their entirety. For example, one may employ an enzymatic reaction with sialidase, such as, for example, Arthrobacter ureafacens sialidase. The conditions of such a reaction are generally described in the U.S. Pat. No. 5,831,077, which is hereby incorporated by reference in its entirety. Other non-limiting examples of suitable enzymes are neuraminidase and N-Glycosidase F, as described in Schloemer et al., J. Virology, 15(4), 882-893 (1975) and in Leibiger et al., Biochem J., 338, 529-538 (1999), respectively. Desialylated antibodies may be further purified by using affinity chromatography. Alternatively, one may employ methods to increase the level of sialyation, such as by employing sialytransferase enzymes. Conditions of such a reaction are generally described in Basset et al., Scandinavian Journal of Immunology, 51(3), 307-311 (2000).

Another modification of the antibodies herein that is contemplated by the invention is pegylation. An antibody can be pegylated to, for example, increase the biological (e.g., serum) half life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al. As such, the methods of pegylation described here also apply the peptidic molecules of the invention described below.

### Antibody Fragments and Antibody Mimetics

The instant invention is not limited to traditional antibodies and may be practiced through the use of antibody fragments and antibody mimetics. As detailed below, a wide variety of antibody fragment and antibody mimetic technologies have now been developed and are widely known in the art. While a number of these technologies, such as domain antibodies, Nanobodies, and UniBodies make use of fragments of, or other modifications to, traditional antibody structures, there are also alternative technologies, such as Adnectins, Affibodies, DARPins, Anticalins, Avimers, and Versabodies that employ binding structures that, while they mimic traditional antibody binding, are generated from and function via distinct mechanisms. Some of these alternative structures are reviewed in Gill and Damle (2006) 17: 653-658.

Domain Antibodies (dAbs) are the smallest functional binding units of antibodies, corresponding to the variable regions of either the heavy (VH) or light (VL) chains of human antibodies. Domain Antibodies have a molecular weight of approximately 13 kDa. Domantis has developed a series of large and highly functional libraries of fully human VH and VL dAbs (more than ten billion different sequences in each library), and uses these libraries to select dAbs that are specific to therapeutic targets. In contrast to many conventional antibodies, domain antibodies are well expressed in bacterial, yeast, and mammalian cell systems. Further details of domain antibodies and methods of production thereof may be obtained by reference to U.S. Pat. Nos. 6,291,158; 6,582,915; 6,593,081; 6,172,197; 6,696,245; U.S. Serial No. 2004/0110941; European patent application No. 1433846 and European Patents 0368684 & 0616640; WO05/035572, WO04/101790, WO04/081026, WO04/058821, WO04/003019 and WO03/002609, each of which is herein incorporated by reference in its entirety.

Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, the cloned and isolated VHH domain is a perfectly stable polypeptide harbouring the full antigen-binding capacity of the original heavy-chain antibody. Nanobodies have a high homology with the VH domains of human antibodies and can be further humanized without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies, Nanobodies show high target specificity, high affinity for their target and low inherent toxicity. However, like small molecule drugs they can inhibit enzymes and readily access receptor clefts. Furthermore, Nanobodies are extremely stable, can be administered by means other than injection (see, e.g., WO 04/041867, which is herein incorporated by reference in its entirety) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula or Pichia) (see, e.g., U.S. Pat. No. 6,838,254, which is herein incorporated by reference in its entirety). The production process is scalable and multi-kilogram quantities of Nanobodies have been produced. Because Nanobodies exhibit a superior stability compared with conventional antibodies, they can be formulated as a long shelf-life, ready-to-use solution.

The Nanoclone method (see, e.g., WO 06/079372, which is herein incorporated by reference in its entirety) is a proprietary method for generating Nanobodies against a desired target, based on automated high-throughout selection of B-cells and could be used in the context of the instant invention.

UniBodies are another antibody fragment technology, however this one is based upon the removal of the hinge region of IgG4 antibodies. The deletion of the hinge region results in a molecule that is essentially half the size of traditional IgG4 antibodies and has a univalent binding region rather than the bivalent binding region of IgG4 antibodies. It is also well known that IgG4 antibodies are inert and thus do not interact with the immune system, which may be advantageous for the treatment of diseases where an immune response is not desired, and this advantage is passed onto UniBodies. For example, UniBodies may function to inhibit or silence, but not kill, the cells to which they are bound. Additionally, UniBody binding to cancer cells do not stimulate them to proliferate. Furthermore, because UniBodies are about half the size of traditional IgG4 antibodies, they may show better distribution over larger solid tumors with potentially advantageous efficacy. UniBodies are cleared from the body at a similar rate to whole IgG4 antibodies and are able to bind with a similar affinity for their antigens as whole antibodies. Further details of UniBodies may be obtained by reference to patent application WO2007/059782, which is herein incorporated by reference in its entirety.

Adnectin molecules are engineered binding proteins derived from one or more domains of the fibronectin protein. Fibronectin exists naturally in the human body. It is present in the extracellular matrix as an insoluble glycoprotein dimer and also serves as a linker protein. It is also present in soluable form in blood plasma as a disulphide linked dimer. The plasma form of fibronectin is synthesized by liver cells (hepatocytes), and the ECM form is made by chondrocytes, macrophages, endothelial cells, fibroblasts, and some cells of the epithelium (see Ward M., and Marcey, D., callutheran.edu/Academic_Programs/Departments/BioDev/omm/fibro/fibro.htm). As mentioned previously, fibronectin may function naturally as a cell adhesion molecule, or it may mediate the interaction of cells by making contacts in the extracellular matrix. Typically, fibronectin is made of three different protein modules, type I, type II, and type III modules. For a review of the structure of function of the fibronectin, see Pankov and Yamada (2002) J Cell Sci., 115(Pt 20):3861-3, Hohenester and Engel (2002) 21:115-128, and Lucena et al. (2007) Invest Clin. 48:249-262.

In a preferred embodiment, adnectin molecules are derived from the fibronectin type III domain by altering the native protein which is composed of multiple beta strands distributed between two beta sheets. Depending on the originating tissue, fibronecting may contain multiple type III domains which may be denoted, e.g., ¹Fn3, ²Fn3, ³Fn3, etc. The ¹⁰Fn3 domain contains an integrin binding motif and further contains three loops which connect the beta strands. These loops may be thought of as corresponding to the antigen binding loops of the IgG heavy chain, and they may be altered by methods discussed below to specifically bind a target of interest, e.g., an asymmetric contact interface of a RTK, such as a fibroblast growth factor receptor. Preferably, a fibronectin type III domain useful for the purposes of this invention is a sequence which exhibits a sequence identity of at least 30%, at least 40%, at least 50%, at least 60%, at least 70%, at least 80%, at least 90%, or at least 95% to the sequence encoding the structure of the fibronectin type III molecule which can be accessed from the Protein Data Bank (PDB, rcsb.org/pdb/home/home.do) with the accession code: 1ttg. Adnectin molecules may also be derived from polymers of ¹⁰Fn3 related molecules rather than a simple monomeric ¹⁰Fn3 structure.

Although the native ¹⁰Fn3 domain typically binds to integrin, ¹⁰Fn3 proteins adapted to become adnectin molecules are altered so to bind antigens of interest, e.g., an asymmetric contact interface of a RTK, such as a fibroblast growth factor receptor. In one embodiment, the alteration to the ¹⁰Fn3 molecule comprises at least one mutation to a beta strand. In a preferred embodiment, the loop regions which connect the beta strands of the ¹⁰Fn3 molecule are altered to bind to an asymmetric contact interface of a human receptor tyrosine kinase, e.g., a FGFR or a type IV receptor tyrosine kinase.

The alterations in the ¹⁰Fn3 may be made by any method known in the art including, but not limited to, error prone PCR, site-directed mutagenesis, DNA shuffling, or other types of recombinational mutagenesis which have been referenced herein. In one example, variants of the DNA encoding the ¹⁰Fn3 sequence may be directly synthesized in vitro, and later transcribed and translated in vitro or in vivo. Alternatively, a natural ¹⁰Fn3 sequence may be isolated or cloned from the genome using standard methods (as performed, e.g., in U.S. Pat. Application No. 20070082365), and then mutated using mutagenesis methods known in the art.

In one embodiment, a target protein, e.g., an asymmetric contact interface of a RTK, such as a fibroblast growth factor receptor, may be immobilized on a solid support, such as a column resin or a well in a microtiter plate. The target is then contacted with a library of potential binding proteins. The library may comprise ¹⁰Fn3 clones or adnectin molecules derived from the wild type ¹⁰Fn3 by mutagenesis/randomization of the ¹⁰Fn3 sequence or by mutagenesis/randomization of the ¹⁰Fn3 loop regions (not the beta strands). In a preferred embodiment the library may be an RNA-protein fusion library generated by the techniques described in Szostak et al., U.S. Ser. No. 09/007,005 and 09/247,190; Szostak et al., WO989/31700; and Roberts & Szostak (1997) 94:12297-12302. The library may also be a DNA-protein library (e.g., as described in Lohse, U.S. Ser. No. 60/110,549, U.S. Ser. No. 09/459,190, and WO 00/32823). The fusion library is then incubated with the immobilized target (e.g., the asymmetric contact interface of an RTK) and the solid support is washed to remove non-specific binding moieties. Tight binders are then eluted under stringent conditions and PCR is used to amply the genetic information or to create a new library of binding molecules to repeat the process (with or without additional mutagenesis). The selection/mutagenesis process may be repeated until binders with sufficient affinity to the target are obtained. Adnectin molecules for use in the present invention may be engineered using the PROfusion™ technology employed by Adnexus, a Briston-Myers Squibb company. The PROfusion technology was created based on the techniques referenced above (e.g., Roberts & Szostak (1997) 94:12297-12302). Methods of generating libraries of altered ¹⁰Fn3 domains and selecting appropriate binders which may be used with the present invention are described fully in the following U.S. patent and patent application documents and are incorporated herein by reference: U.S. Pat. Nos. 7,115,396; 6,818,418; 6,537,749; 6,660,473; 7,195,880; 6,416,950; 6,214,553; 6,623,926; 6,312,927; 6,602,685; 6,518,018; 6,207,446; 6,258,558; 6,436,665; 6,281,344; 7,270,950; 6,951,725; 6,846,655; 7,078,197; 6,429,300; 7,125,669; 6,537,749; 6,660,473; and U.S. Pat. Application Nos. 20070082365; 20050255548; 20050038229; 20030143616; 20020182597; 20020177158; 20040086980; 20040253612; 20030022236; 20030013160; 20030027194; 20030013110; 20040259155; 20020182687; 20060270604; 20060246059; 20030100004; 20030143616; and 20020182597. The generation of diversity in fibronectin type III domains, such as ¹⁰Fn3, followed by a selection step may be accomplished using other methods known in the art such as phage display, ribosome display, or yeast surface display, e.g., Lipov\check{s}ek et al. (2007) Journal of Molecular Biology 368: 1024-1041; Sergeeva et al. (2006) Adv Drug Deliv Rev. 58:1622-1654; Petty et al. (2007) Trends Biotechnol. 25: 7-15; Rothe et al. (2006) Expert Opin Biol Ther. 6:177-187; and Hoogenboom (2005) Nat. Biotechnol. 23:1105-1116.

It should be appreciated by one of skill in the art that the methods references cited above may be used to derive antibody mimics from proteins other than the preferred ¹⁰Fn3 domain. Additional molecules which can be used to generate antibody mimics via the above referenced methods include, without limitation, human fibronectin modules ¹Fn3-⁹Fn3 and ¹¹Fn3-¹⁷Fn3 as well as related Fn3 modules from non-human animals and prokaryotes. In addition, Fn3 modules from other proteins with sequence homology to ¹⁰Fn3, such as tenascins and undulins, may also be used. Other exemplary proteins having immunoglobulin-like folds (but with sequences that are unrelated to the V_{H} domain) include N-cadherin, ICAM-2, titin, GCSF receptor, cytokine receptor, glycosidase inhibitor, E-cadherin, and antibiotic chromoprotein. Further domains with related structures may be derived from myelin membrane adhesion molecule PO, CD8, CD4, CD2, class I MHC, T-cell antigen receptor, CD1, C2 and I-set domains of VCAM-1, I-set immunoglobulin fold of myosin-binding protein C, I-set immunoglobulin fold of myosin-binding protein H, I-set immunoglobulin-fold of telokin, telikin, NCAM, twitchin, neuroglian, growth hormone receptor, erythropoietin receptor, prolactin receptor, GC-SF receptor, interferon-gamma receptor, beta-galactosidase/glucuronidase, beta-glucuronidase, and transglutaminase. Alternatively, any other protein that includes one or more immunoglobulin-like folds may be utilized to create a adnecting like binding moiety. Such proteins may be identified, for example, using the program SCOP (Murzin et al., J. Mol. Biol. 247:536 (1995); Lo Conte et al., Nucleic Acids Res. 25:257 (2000).

An aptamer is another type of antibody-mimetic which is encompassed by the present invention. Aptamers are typically small nucleotide polymers that bind to specific molecular targets. Aptamers may be single or double stranded nucleic acid molecules (DNA or RNA), although DNA based aptamers are most commonly double stranded. There is no defined length for an aptamer nucleic acid; however, aptamer molecules are most commonly between 15 and 40 nucleotides long.

Aptamers often form complex three-dimensional structures which determine their affinity for target molecules. Aptamers can offer many advantages over simple antibodies, primarily because they can be engineered and amplified almost entirely in vitro. Furthermore, aptamers often induce little or no immune response. Aptamers may be generated using a variety of techniques, but were originally developed using in vitro selection (Ellington and Szostak (1990) Nature, 346(6287):818-22) and the SELEX method (systematic evolution of ligands by exponential enrichment) (Schneider et al. 1992. J Mol Biol. 228(3):862-9) the contents of which are incorporated herein by reference. Other methods to make and uses of aptamers have been published including Klussmann. The Aptamer Handbook: Functional Oligonucleotides and Their Applications. ISBN: 978-3-527-31059-3; Ulrich et al. 2006, Comb Chem High Throughput Screen 9(8):619-32; Cerchia and de Franciscis, 2007, Methods Mol. Biol. 361:187-200; Ireson and Kelland. 2006, Mol Cancer Ther., 2006 5(12):2957-62; U.S. Pat. Nos. 5,582,981; 5,840,867; 5,756,291; 6,261,783; 6,458,559; 5,792,613; 6,111,095; and U.S. patent application Ser. Nos. 11/482,671; 11/102,428; 11/291,610; and 10/627,543 which are all incorporated herein by reference.

The SELEX method is clearly the most popular and is conducted in three fundamental steps. First, a library of candidate nucleic acid molecules is selected from for binding to specific molecular target. Second, nucleic acids with sufficient affinity for the target are separated from non-binders. Third, the bound nucleic acids are amplified, a second library is formed, and the process is repeated. At each repetition, aptamers are chosen which have higher and higher affinity for the target molecule. SELEX methods are described more fully in the following publications, which are incorporated herein by reference: Bugaut et al. 2006. 4(22):4082-8; Stoltenburg et al. 2007 Biomol Eng. 2007 24(4):381-403; and Gopinath, 2007, Anal Bioanal Chem. 2007. 387(1):171-82.

An “aptamer” of the invention also been includes aptamer molecules made from peptides instead of nucleotides. Peptide aptamers share many properties with nucleotide aptamers (e.g., small size and ability to bind target molecules with high affinity) and they may be generated by selection methods that have similar principles to those used to generate nucleotide aptamers, for example Baines and Colas. 2006. Drug Discov Today. 11(7-8):334-41; and Bickle et al. 2006. Nat. Protoc. 1(3):1066-91 which are incorporated herein by reference.

Affibody molecules represent a new class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (Nord K, Gunneriusson E, Ringdahl J, Stahl S, Uhlen M, Nygren P A, Binding proteins selected from combinatorial libraries of an α-helical bacterial receptor domain, Nat Biotechnol 1997; 15:772-7. Ronmark J, Gronlund H, Uhlen M, Nygren P A, Human immunoglobulin A (IgA)-specific ligands from combinatorial engineering of protein A, Eur J Biochem 2002; 269:2647-55). The simple, robust structure of Affibody molecules in combination with their low molecular weight (6 kDa), make them suitable for a wide variety of applications, for instance, as detection reagents (Ronmark J, Hansson M, Nguyen T, et al, Construction and characterization of affibody-Fc chimeras produced in Escherichia coli, J Immunol Methods 2002; 261:199-211) and to inhibit receptor interactions (Sandstorm K, Xu Z, Forsberg G, Nygren P A, Inhibition of the CD28-CD80 co-stimulation signal by a CD28-binding Affibody ligand developed by combinatorial protein engineering, Protein Eng 2003; 16:691-7). In some embodiments, affibodies have been altered to remain as intracellular antibodies by extending the antibody sequence with KDEL to make it resident in the secretory compartments (see, e.g., Vernet et al. (2009) New Biotechnology, 25(6):417-423). Further details of Affibodies and methods of production thereof may be obtained by reference to U.S. Pat. No. 5,831,012 which is herein incorporated by reference in its entirety.

DARPins (Designed Ankyrin Repeat Proteins) are one example of an antibody mimetic DRP (Designed Repeat Protein) technology that has been developed to exploit the binding abilities of non-antibody polypeptides. Repeat proteins such as ankyrin or leucine-rich repeat proteins, are ubiquitous binding molecules, which occur, unlike antibodies, intra- and extracellularly. Their unique modular architecture features repeating structural units (repeats), which stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains.

DARPins can be produced in bacterial expression systems at very high yields and they belong to the most stable proteins known. Highly specific, high-affinity DARPins to a broad range of target proteins, including human receptors, cytokines, kinases, human proteases, viruses and membrane proteins, have been selected. DARPins having affinities in the single-digit nanomolar to picomolar range can be obtained.

DARPins have been used in a wide range of applications, including ELISA, sandwich ELISA, flow cytometric analysis (FACS), immunohistochemistry (IHC), chip applications, affinity purification or Western blotting. DARPins also proved to be highly active in the intracellular compartment for example as intracellular marker proteins fused to green fluorescent protein (GFP). DARPins were further used to inhibit viral entry with IC50 in the pM range. DARPins are not only ideal to block protein-protein interactions, but also to inhibit enzymes. Proteases, kinases and transporters have been successfully inhibited, most often an allosteric inhibition mode. Very fast and specific enrichments on the tumor and very favorable tumor to blood ratios make DARPins well suited for in vivo diagnostics or therapeutic approaches.

Additional information regarding DARPins and other DRP technologies can be found in U.S. Patent Application Publication No. 2004/0132028 and International Patent Application Publication No. WO 02/20565, both of which are hereby incorporated by reference in their entirety.

Anticalins are an additional antibody mimetic technology, however in this case the binding specificity is derived from lipocalins, a family of low molecular weight proteins that are naturally and abundantly expressed in human tissues and body fluids. Lipocalins have evolved to perform a range of functions in vivo associated with the physiological transport and storage of chemically sensitive or insoluble compounds. Lipocalins have a robust intrinsic structure comprising a highly conserved β-barrel which supports four loops at one terminus of the protein. These loops form the entrance to a binding pocket and conformational differences in this part of the molecule account for the variation in binding specificity between individual lipocalins.

While the overall structure of hypervariable loops supported by a conserved β-sheet framework is reminiscent of immunoglobulins, lipocalins differ considerably from antibodies in terms of size, being composed of a single polypeptide chain of 160-180 amino acids which is marginally larger than a single immunoglobulin domain.

Lipocalins are cloned and their loops are subjected to engineering in order to create Anticalins. Libraries of structurally diverse Anticalins have been generated and Anticalin display allows the selection and screening of binding function, followed by the expression and production of soluble protein for further analysis in prokaryotic or eukaryotic systems. Studies have successfully demonstrated that Anticalins can be developed that are specific for virtually any human target protein can be isolated and binding affinities in the nanomolar or higher range can be obtained.

Anticalins can also be formatted as dual targeting proteins, so-called Duocalins. A Duocalin binds two separate therapeutic targets in one easily produced monomeric protein using standard manufacturing processes while retaining target specificity and affinity regardless of the structural orientation of its two binding domains.

Modulation of multiple targets through a single molecule is particularly advantageous in diseases known to involve more than a single causative factor. Moreover, bi- or multivalent binding formats such as Duocalins have significant potential in targeting cell surface molecules in disease, mediating agonistic effects on signal transduction pathways or inducing enhanced internalization effects via binding and clustering of cell surface receptors. Furthermore, the high intrinsic stability of Duocalins is comparable to monomeric Anticalins, offering flexible formulation and delivery potential for Duocalins.

Additional information regarding Anticalins can be found in U.S. Pat. No. 7,250,297 and International Patent Application Publication No. WO 99/16873, both of which are hereby incorporated by reference in their entirety.

Another antibody mimetic technology useful in the context of the instant invention are Avimers. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. Other potential advantages include simple and efficient production of multitarget-specific molecules in Escherichia coli, improved thermostability and resistance to proteases. Avimers with sub-nanomolar affinities have been obtained against a variety of targets.

Additional information regarding Avimers can be found in U.S. Patent Application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

Versabodies are another antibody mimetic technology that could be used in the context of the instant invention. Versabodies are small proteins of 3-5 kDa with >15% cysteines, which form a high disulfide density scaffold, replacing the hydrophobic core that typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T-cell epitopes, because the residues that contribute most to MHC presentation are hydrophobic. All four of these properties are well-known to affect immunogenicity, and together they are expected to cause a large decrease in immunogenicity.

The inspiration for Versabodies comes from the natural injectable biopharmaceuticals produced by leeches, snakes, spiders, scorpions, snails, and anemones, which are known to exhibit unexpectedly low immunogenicity. Starting with selected natural protein families, by design and by screening the size, hydrophobicity, proteolytic antigen processing, and epitope density are minimized to levels far below the average for natural injectable proteins.

Given the structure of Versabodies, these antibody mimetics offer a versatile format that includes multi-valency, multi-specificity, a diversity of half-life mechanisms, tissue targeting modules and the absence of the antibody Fc region. Furthermore, Versabodies are manufactured in E. coli at high yields, and because of their hydrophilicity and small size, Versabodies are highly soluble and can be formulated to high concentrations. Versabodies are exceptionally heat stable (they can be boiled) and offer extended shelf-life.

Additional information regarding Versabodies can be found in U.S. Patent Application Publication No. 2007/0191272 which is hereby incorporated by reference in its entirety.

SMIPs™ (Small Modular ImmunoPharmaceuticals-Trubion Pharmaceuticals) engineered to maintain and optimize target binding, effector functions, in vivo half life, and expression levels. SMIPS consist of three distinct modular domains. First they contain a binding domain which may consist of any protein which confers specificity (e.g., cell surface receptors, single chain antibodies, soluble proteins, etc). Secondly, they contain a hinge domain which serves as a flexible linker between the binding domain and the effector domain, and also helps control multimerization of the SMIP drug. Finally, SMIPS contain an effector domain which may be derived from a variety of molecules including Fc domains or other specially designed proteins. The modularity of the design, which allows the simple construction of SMIPs with a variety of different binding, hinge, and effector domains, provides for rapid and customizable drug design.

More information on SMIPs, including examples of how to design them, may be found in Zhao et al. (2007) Blood 110:2569-77 and the following U.S. Pat. App. Nos. 20050238646; 20050202534; 20050202028; 20050202023; 20050202012; 20050186216; 20050180970; and 20050175614, each of which are incorporated herein by reference in their entirety.

The detailed description of antibody fragment and antibody mimetic technologies provided above is not intended to be a comprehensive list of all technologies that could be used in the context of the instant specification. For example, and also not by way of limitation, a variety of additional technologies including alternative polypeptide-based technologies, such as fusions of complimentary determining regions as outlined in Qui et al., Nature Biotechnology, 25(8) 921-929 (2007), which is hereby incorporated by reference in its entirety, as well as nucleic acid-based technologies, such as the RNA aptamer technologies described in U.S. Pat. Nos. 5,789,157, 5,864,026, 5,712,375, 5,763,566, 6,013,443, 6,376,474, 6,613,526, 6,114,120, 6,261,774, and 6,387,620, all of which are hereby incorporated by reference, could be used in the context of the instant invention.

### Antibody Physical Properties

The antibodies of the present invention, which bind to an asymmetric contact interface of a RTK, may be further characterized by the various physical properties. Various assays may be used to detect and/or differentiate different classes of antibodies based on these physical properties.

In some embodiments, antibodies of the present invention may contain one or more glycosylation sites in either the light or heavy chain variable region. The presence of one or more glycosylation sites in the variable region may result in increased immunogenicity of the antibody or an alteration of the pK of the antibody due to altered antigen binding (Marshall et al (1972) Annu Rev Biochem 41:673-702; Gala F A and Morrison SL (2004) J Immunol 172:5489-94; Wallick et al (1988) J Exp Med 168:1099-109; Spiro R G (2002) Glycobiology 12:43 R-56R; Parekh et al (1985) Nature 316:452-7; Mimura et al. (2000) Mol Immunol 37:697-706). Glycosylation has been known to occur at motifs containing an N—X—S/T sequence. Variable region glycosylation may be tested using a Glycoblot assay, which cleaves the antibody to produce a Fab, and then tests for glycosylation using an assay that measures periodate oxidation and Schiff base formation. Alternatively, variable region glycosylation may be tested using Dionex light chromatography (Dionex-LC), which cleaves saccharides from a Fab into monosaccharides and analyzes the individual saccharide content. In some instances, it may be preferred to have an antibody that does not contain variable region glycosylation. This can be achieved either by selecting antibodies that do not contain the glycosylation motif in the variable region or by mutating residues within the glycosylation motif using standard techniques well known in the art.

Each antibody will have a unique isoelectric point (pI), but generally antibodies will fall in the pH range of between 6 and 9.5. The pI for an IgG1 antibody typically falls within the pH range of 7-9.5 and the pI for an IgG4 antibody typically falls within the pH range of 6-8. Antibodies may have a pI that is outside this range. Although the effects are generally unknown, there is speculation that antibodies with a pI outside the normal range may have some unfolding and instability under in vivo conditions. The isoelectric point may be tested using a capillary isoelectric focusing assay, which creates a pH gradient and may utilize laser focusing for increased accuracy (Janini et al (2002) Electrophoresis 23:1605-11; Ma et al. (2001) Chromatographia 53:S75-89; Hunt et al (1998) J Chromatogr A 800:355-67). In some instances, it is preferred to have an antibody that contains a pI value that falls in the normal range. This can be achieved either by selecting antibodies with a pI in the normal range, or by mutating charged surface residues using standard techniques well known in the art.

Each antibody will have a melting temperature that is indicative of thermal stability (Krishnamurthy R and Manning M C (2002) Curr Pharm Biotechnol 3:361-71). A higher thermal stability indicates greater overall antibody stability in vivo. The melting point of an antibody may be measure using techniques such as differential scanning calorimetry (Chen et al (2003) Pharm Res 20:1952-60; Ghirlando et al (1999) Immunol Lett 68:47-52). T_{M1} indicates the temperature of the initial unfolding of the antibody. T_{M2} indicates the temperature of complete unfolding of the antibody. Generally, it is preferred that the T_{M1} of an antibody of the present invention is greater than 60° C., preferably greater than 65° C., even more preferably greater than 70° C. Alternatively, the thermal stability of an antibody may be measure using circular dichroism (Murray et al. (2002) J. Chromatogr Sci 40:343-9).

In a preferred embodiment, antibodies that do not rapidly degrade may be desired. Fragmentation of an antibody may be measured using capillary electrophoresis (CE) and MALDI-MS, as is well understood in the art (Alexander A J and Hughes D E (1995) Anal Chem 67:3626-32).

In another preferred embodiment, antibodies are selected that have minimal aggregation effects. Aggregation may lead to triggering of an unwanted immune response and/or altered or unfavorable pharmacokinetic properties. Generally, antibodies are acceptable with aggregation of 25% or less, preferably 20% or less, even more preferably 15% or less, even more preferably 10% or less and even more preferably 5% or less. Aggregation may be measured by several techniques well known in the art, including size-exclusion column (SEC) high performance liquid chromatography (HPLC), and light scattering to identify monomers, dimers, trimers or multimers.

### Production of Polyclonal Antibodies of the Invention

Polyclonal antibodies of the present invention can be produced by a variety of techniques that are well known in the art. Polyclonal antibodies are derived from different B-cell lines and thus may recognize multiple epitopes on the same antigen. Polyclonal antibodies are typically produced by immunization of a suitable mammal with the antigen of interest, e.g., an asymmetric contact interface of an RTK. Animals often used for production of polyclonal antibodies are chickens, goats, guinea pigs, hamsters, horses, mice, rats, sheep, and, most commonly, rabbit. Standard methods to produce polyclonal antibodies are widely known in the art and can be combined with the methods of the present invention (e.g., research.cm.utexas.eduibkitto/Kittolabpage/Protocols/Immunology/PAb.html; U.S. Pat. Nos. 4,719,290, 6,335,163, 5,789,208, 2,520,076, 2,543,215, and 3,597,409, the entire contents of which are incorporated herein by reference.

### Production of Monoclonal Antibodies of the Invention

Monoclonal antibodies (mAbs) of the present invention can be produced by a variety of techniques, including conventional monoclonal antibody methodology e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes. It should be noted that antibodies (monoclonal or polyclonal) or antigen binding portions thereof, may be raised to any epitope on an asymmetric contact interface of a RTK, such as a fibroblast growth factor receptor, to the concensus sequences discussed herein, or to any conformational, discontinuous, or linear epitopes described herein.

Several methods known in the art are useful for specifically selecting an antibody or antigen binding fragment thereof that specifically binds a discontinuous epitope of interest. For example, the techniques disclosed in U.S. Publication No. 2005/0169925, the entire contents of which are incorporated herein by reference, allow for the selection of an antibody which binds to two different peptides within a protein sequence. Such methods may be used in accordance with the present invention to specifically target the conformational and discontinuous epitopes disclosed herein. If the conformational epitope is a protein secondary structure, such structures often form easily in smaller peptides (e.g., <50 amino acids). Thus, immunizing an animal with smaller peptides could capture some conformational epitopes. Alternatively, two small peptides which comprise a conformational epitope may be connected via a flexible linker (e.g., polyglycol, or a stretch of polar, uncharged amino acids). The linker will allow the peptides to explore various interaction orientations. Immunizing with this construct, followed by appropriate screening could allow for identification of antibodies directed to a conformational epitope. In a preferred embodiment, peptides to specific conformational or linear epitopes may be generated by immunizing an animal with a particular domain of an RTK (e.g., an asymmetric contact interface of an RTK) and subsequently screening for antibodies which bind the epitope of interest. In one embodiment cryoelectron microscopy (Jiang et al. (2008) Nature 451, 1130-1134; Joachim (2006) Oxford University Press ISBN:0195182189) may be used to identify the epitopes bound by an antibody or antigen binding fragment of the invention. In another embodiment, the RTK or a domain thereof may be crystallized with the bound antibody or antigen binding fragment thereof and analyzed by X-ray crystallography to determine the precise epitopes that are bound. In addition, epitopes may be mapped by replacing portions of an RTK sequence with the corresponding sequences from mouse or another species. Antibodies directed to epitopes of interest will selectively bind the human sequence regions and, thus, it is possible to sequentially map target epitopes. This technique of chimera based epitope mapping has been used successfully to identify epitopes in various settings (see Henriksson and Pettersson (1997) Journal of Autoimmunity. 10(6):559-568; Netzer et al. (1999) J Biol. Chem. 1999 Apr. 16; 274(16):11267-74; Hsia et al. (1996) Mol. Microbiol. 19, 53-63, the entire contents of which are incorporated herein by reference).

It is believed that the epitopes of interest in target RTKs (e.g., fibroblast growth factor receptors) are not glycosylated. However, if an RTK of interest is glycosylated, antibodies or antigen binding portions thereof (and other moieties of the invention), may be raised such that they bind to the relevant amino acid and/or sugar residues. For example, it is known in the art that the Kit protein has at least 10 sites for potential N-linked glycosylation (Morstyn, Foote, Lieschke (2004) Hematopoietic Growth Factors in Oncology: Basic Science and Clinical Therapeutics. Humana Press. ISBN:1588293025). It is further thought that Kit may exhibit O-linked glycosylation as well as attachment to sialic acid residues (Wypych J, et al. (1995) Blood, 85(1):66-73). Thus, it is contemplated that antibodies or antigen binding portions thereof (and other moieties of the invention), may be raised such that they also bind to sugar residues which may be attached to any epitope identified herein. For this purpose, an antigenic peptide of interest may be produced in an animal cell such that it gets properly glycosylated and the glycosylated antigenic peptide may then be used to immunize an animal. Suitable cells and techniques for producing glycosylated peptides are known in the art and described further below (see, for example, the technologies available from GlycoFi, Inc., Lebanon, N.H. and BioWa; Princeton, N.J.). The proper glycosylation of a peptide may be tested using any standard methods such as isoelectric focusing (IEF), acid hydrolysis (to determine monosaccharide composition), chemical or enzymatic cleavage, and mass spectrometry (MS) to identify glycans. The technology offered by Procognia (procognia.com) which uses a lectin-based array to speed up glycan analysis may also be used. O-glycosylation specifically may be detected using techniques such as reductive alkaline cleavage or “beta elimination”, peptide mapping, liquid chromatography, and mass spectrometry or any combination of these techniques.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.). Alternatively, a humanized antibody may be designed at the DNA or protein level, given knowledge of human and non-human sequences. Such antibodies may be directly synthesized chemically, or the DNA may be synthesized and expressed in vitro or in vivo to produce a humanized antibody.

In a preferred embodiment, the antibodies of the invention are human monoclonal antibodies. Such human monoclonal antibodies directed against an asymmetric contact interface of an RTK, e.g. a fibroblast growth factor receptor, can be generated using transgenic or transchromosomic mice carrying parts of the human immune system rather than the mouse system. These transgenic and transchromosomic mice include mice referred to herein as HuMAb mice and KM Mice™, respectively, and are collectively referred to herein as “human Ig mice.”

The HuMAb mouse® (Medarex, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368 (6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546). The preparation and use of HuMab mice, and the genomic modifications carried by such mice, is further described in Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6: 579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.

In another embodiment, human antibodies of the invention can be raised using a mouse that carries human immunoglobulin sequences on transgenes and transchomosomes, such as a mouse that carries a human heavy chain transgene and a human light chain transchromosome. Such mice, referred to herein as “KM Mice™”, are described in detail in PCT Publication WO 02/43478 to Ishida et al.

Still further, alternative transgenic animal systems expressing human immunoglobulin genes are available in the art and can be used to raise the antibodies of the invention. For example, an alternative transgenic system referred to as the Xenomouse (Abgenix, Inc.) can be used; such mice are described in, for example, U.S. Pat. Nos. 5,939,598; 6,075,181; 6,114,598; 6, 150,584 and 6,162,963 to Kucherlapati et al.

Moreover, alternative transchromosomic animal systems expressing human immunoglobulin genes are available in the art and can be used to raise the antibodies of the invention. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise the antibodies of the invention.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In another embodiment, antibodies of the invention may be raised using well known phage display techniques, as described in Marks, J. D., et al. ((1991). J. Mol. Biol. 222, 581), Nissim, A., et al. ((1994). EMBO J. 13, 692) and U.S. Pat. Nos. 6,794,132; 6,562,341; 6,057,098; 5,821,047; and 6,512,097.

In a further embodiment, antibodies of the present invention may be found using yeast cell surface display technology as described, for example, in U.S. Pat. Nos. 6,423,538; 6,300,065; 6,696,251; 6,699,658.

### Generation of Hybridomas Producing Human Monoclonal Antibodies of the Invention

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3X63-Ag8. 653 nonsecreting mouse myeloma cells (ATCC, CRL 1580) with 50% PEG. Alternatively, the single cell suspension of splenic lymphocytes from immunized mice can be fused using an electric field based electrofusion method, using a CytoPulse large chamber cell fusion electroporator (CytoPulse Sciences, Inc., Glen Burnie Md.). Cells are plated at approximately 2×10⁵ in flat bottom microtiter plate, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The monoclonal antibodies can be aliquoted and stored at −80° C.

### Generation of Transfectomas Producing Monoclonal Antibodies of the Invention

Antibodies of the invention also can be produced in a host cell transfectoma (a type of hybridoma) using, for example, a combination of recombinant DNA techniques and gene transfection methods as is well known in the art (e.g., Morrison, S. (1985) Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the described antibodies can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{K} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SR promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

### Characterization of Antibody Binding to an Asymmetric Contact Interface of a RTK

Antibodies of the invention can be tested for binding to an asymmetric contact interface of a RTK (or any chosen region such as the concensus sequences discussed herein) by, for example, standard ELISA. Briefly, microtiter plates are coated with the purified asymmetric contact interface (or a preferred receptor domain) at 0.25 μg/ml in PBS, and then blocked with 5% bovine serum albumin in PBS. Dilutions of antibody (e.g., dilutions of plasma from immunized mice, e.g., mice immunized with the asymmetric contact interface of an RTK) are added to each well and incubated for 1-2 hours at 37° C. The plates are washed with PBS/Tween and then incubated with secondary reagent (e.g., for human antibodies, a goat-anti-human IgG Fc-specific polyclonal reagent) conjugated to alkaline phosphatase for 1 hour at 37° C. After washing, the plates are developed with pNPP substrate (1 mg/ml), and analyzed at OD of 405-650. Preferably, mice which develop the highest titers will be used for fusions.

An ELISA assay as described above can also be used to screen for hybridomas that show positive reactivity with immunogen. Hybridomas that bind with high avidity to, e.g., an asymmetric contact interface of an RTK, are subcloned and further characterized. One clone from each hybridoma, which retains the reactivity of the parent cells (by ELISA), can be chosen for making a 5-10 vial cell bank stored at −140° C., and for antibody purification.

To purify anti-RTK antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The monoclonal antibodies can be aliquoted and stored at −80° C.

To determine if the selected monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using RTK coated ELISA plates coated with an asymmetric contact interface of a RTK (e.g., a fibroblast growth factor receptor) as described above. Biotinylated mAb binding can be detected with a strep-avidin-alkaline phosphatase probe.

To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-RTK human IgGs can be further tested for reactivity with an asymmetric contact interface of a RTK or a concensus sequence presented herein by Western blotting. Briefly, an asymmetric contact interface of a RTK, such as a fibroblast growth factor receptor, can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

Epitope mapping may be employed to determine the binding site of an antibody or antigen binding fragment thereof of the invention. Several methods are available which further allow the mapping of conformational epitopes. For example, the methods disclosed in Timmerman et al. (Mol. Divers. 2004; 8(2):61-77) may be used. Timmerman et al. were able to successfully map discontinuous/conformational epitopes using two novel techniques, Domain Scan and Matrix Scan. The techniques disclosed in Ansong et al. (J Thromb Haemost. 2006. 4(4):842-7) may also be used. Ansong et al. used affinity directed mass spectrometry to map the discontinuous epitope recognized by the antibody R8B 12. In addition, imaging techniques such as Protein Tomography may be used to visualize antibody or peptide binding to target RTKs. Protein Tomography has been used previously to gain insight into molecular interactions, and was used to show that an inhibitory antibody acted by binding domain III of EGFR thereby locking EGFR into an inflexible and inactive conformation (Lammerts et al. Proc Natl Acad Sci USA. 2008.105(16):6109-14). More traditional methods such as site-directed mutagenesis may also be applied to map discontinuous epitopes. Amino acid regions thought to participate in a discontinuous epitope may be selectively mutated and assayed for binding to an antibody or antigen binding fragment thereof of the invention. The inability of the antibody to bind when either region is mutated may indicate that binding is dependent upon both amino acid segments. As noted above, some linear epitopes are characterized by particular three-dimensional structures which must be present in order to bind a moiety of the invention. Such epitopes may be discovered by assaying the binding of the antibody (or another moiety) when the RTK is in its native or folded state and again when the RTK is denatured. An observation that binding occurs only in the folded state would indicate that the epitope is either a linear epitope characterized by a particular folded structure or a discontinuous epitope only present in folded protein.

In addition to the activity assays described herein, Protein Tomography may be used to determine whether an antibody or antigen binding fragment thereof of the invention is able to bind and inactivate a receptor tyrosine kinase. Visualization of the binding interaction may indicate that binding of the antibody may affect the positioning of the asymmetric contact interface or alter or prevent conformational changes in the receptor tyrosine kinase.

### IV. Screening Assays for Identifying Moieties of the Invention

The moieties of the invention may be screened for RTK inhibitory activity using any of the assays described herein and those assays that are well known in the art. For example, assays which may determine receptor internalization, receptor autophosphorylation, and/or kinase signaling may be used to identify moieties which prevent the activation of target RTKs, e.g., a fibroblast growth factor receptor. Screening for new inhibitor moieties may be accomplished by using standard methods known in the art, for example, by employing a phosphoELISA™ procedure (available at Invitrogen) to determine the phosphorylation state of the RTK or a downstream molecule. The phosphorylation state of the receptor, e.g., the FGFR, may be determined using commercially available kits such as, for example, FGFR1Kinase Assay Kit (US Biological; Catalog Number—F4305-15), Human Phospho-FGF R1DuoSet IC Econ Pk, 15 plate (R&D Systems®; Catalog Number—DYC5079E), Phospho-FGFR1 (Y463) & FGFR1Dual Recognition Pair (Novus Biologicals®; Catalog Number—DP0025), C-Kit [pY823] ELISA KIT, HU (BioSource™; Catalog Number—KHO0401); c-KIT [TOTAL] ELISA KIT, HU (BioSource™; Catalog Number—KHO0391). Antibodies, small molecules, and other moieties of the invention may be screened using such kits to determine their RTK inhibitory activity. For example, after treatment with an appropriate ligand and a moiety of the invention, a phosphoELISA™ may be performed to determine the phosphorylation state and, thus, the activation state of a RTK of interest. Moieties of the invention could be identified as those which prevent RTK activation. The Examples below describe assays which involve the detection of RTK activation using anti-phosphotyrosine antibodies. The Examples (including the methods and introduction related thereto) describe further methods used herein to determine the ligand-induced trans autophosphorylation of RTKs.

Since receptor activation may lead to endocytosis and receptor internalization, it is useful, in some embodiments, to determine the ability of moieties of the invention to inhibit target RTKs by measuring their ability to prevent receptor internalization. Receptor internalization assays are well known in the art and described in, for example, Fukunaga et al. (2006) Life Sciences. 80(1):17-23; Bernhagen et al. (2007) Nature Medicine 13, 587-596; natureprotocols.com/2007/04/18/receptor_internalization_assay.php), the entire contents of each of which are incorporated herein by reference. One well-known method to determine receptor internalization is to tag a ligand with a fluororecent protein, e.g., Green Fluororescent Protein (GFP), or other suitable labeling agent. Upon binding of the ligand to the receptor, fluororescence microscopy may be used to visualize receptor internalization. Similarly, a moiety of the invention may be tagged with a labeling agent and fluororescence microscopy may be used to visualize receptor internalization. If the moiety is able to inhibit the activity of the receptor, lessened internalization of fluororescence in the presence of ligand as compared to appropriate controls (e.g., fluorescence may be observed only at the periphery of the cell where the moiety binds the receptor rather than in endosomes or vesicles).

In addition to those mentioned above, various other receptor activation assays are known in the art, any of which may be used to evaluate the function of the moieties of the invention. Further receptor activation assays which may be used in accordance with the present invention are described in U.S. Pat. Nos. 6,287,784; 6,025,145; 5,599,681; 5,766,863; 5,891,650; 5,914,237; 7,056,685; and many scientific publications including, but not limited to: Amir-Zaltsman et al. (2000) Luminescence 15(6):377-80; Nakayama and Parandoosh (1999) Journal of Immunological Methods. 225(1-2), 27, 67-74; Pike et al. (1987) Methods of Enzymology 146: 353-362; Atienza et al. (2005) Journal of Biomolecular Screening. 11(6): 634-643; Hunter et al. (1982). Journal of Biological Chemistry 257(9): 4843-4848; White and Backer (1991) Methods in Enzymology 201: 65-67; Madden et al. (1991) Anal Biochem 199: 210-215; Cleaveland et al. (1990) Analytical Biochemistry 190: 249-253; Lazaro et al. (1991) Analytical Biochemistry 192: 257-261; Hunter and Cooper (1985) Ann Rev Biochem 54: 897-930; Ullrich and Schlessinger (1990) Cell 61: 203-212; Knutson and Buck (1991) Archives of Biochemistry and Biophysics 285(2): 197-204); King et al. (1993) Life Sciences 53: 1465-1472; Wang. (1985) Molecular and Cellular Biology 5(12): 3640-3643; Glenney et al. (1988) Journal of Immunological Methods 109: 277-285; Kamps (1991) Methods in Enzymology 201: 101-110; Kozma et al. (1991) Methods in Enzymology 201: 28-43; Holmes et al. (1992) Science 256: 1205-10; and Corfas et al. (1993) PNAS, USA 90: 1624-1628.

Receptor activation by ligand binding typically initiates subsequent intracellular events, e.g., increases in secondary messengers such as IP₃ which, in turn, releases intracellular stores of calcium ions. Thus, receptor activity may be determined by measuring the quantity of secondary messengers such as IP₃, cyclic nucleotides, intracellular calcium, or phosphorylated signaling molecules such as STAT, PI3K,Grb2, or other possible targets known in the art. U.S. Pat. No. 7,056,685 describes and references several methods which may be used in accordance with the present invention to detect receptor activity and is incorporated herein by reference.

Many of the assays described above, such as receptor internalization assays or receptor activation assays may involve the detection or quantification of a target RTK using immunological binding assays (e.g., when using a radiolabeled antibody to detecting the amount of RTK on the cell surface during a receptor internalization assay). Immunological binding assays are widely described in the art (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991).

Immunoassays such as may be employed in receptor internalization studies, receptor activation studies, or receptor detection assays often use a labeling agent to specifically bind to and label the complex formed by the detecting antibody and the RTK (see U.S. Pat. No. 7,056,685 which is incorporated herein by reference). The labeling agent may itself be the antibody used to detect the receptor (the antibody here may or may not be a moiety of the invention). Alternatively, the labeling agent may be a third agent, such as a secondary or tertiary antibody (e.g., and anti-mouse antibody binding to mouse monoclonal antibody specific for the target RTK). Other proteins capable of specifically binding immunoglobulin constant regions, such as protein A or protein G may also be used as the labeling agent in an immunological binding assay. These proteins exhibit a strong non-immunogenic reactivity with immunoglobulin constant regions from a variety of species (see, e.g., Kronval et al. (1973), J. Immunol. 111:1401-1406; Akerstrom et al. (1985), J. Immunol. 135:2589 2542). The labeling agent can also be modified with a detectable agent, such as biotin, to which another molecule can specifically bind, such as streptavidin. A variety of detectable moieties are well known to those skilled in the art.

Commonly used assays include noncompetitive assays, e.g., sandwich assays, and competitive assays. Commonly used assay formats include Western blots (immunoblots), which are used to detect and quantify the presence of protein in a sample. The particular label or detectable group used in the assay is not a critical aspect of the invention, as long as it does not significantly interfere with the specific binding of the immunoglobulin used to detect the RTK or a moiety of the invention which is designed to bind and inactivate the RTK. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene or latex).

The label may be coupled directly or indirectly to the desired component of the assay according to methods well known in the art. The label can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases.

Fluorescent compounds include fluorescein and its derivatives, rhodamine and its derivatives, dansyl, umbelliferone, and the like. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

In a further aspect of the invention, the moieties of the present invention may bind to epitopes on a target RTK and still allow the receptor tyrosine kinase to dimerize. In this embodiment, the binding of the moiety may affect the positioning, orientation and/or distance between the asymmetric contact interfaces of the two monomers (e.g., the asymmetric contact interfaces of two fibroblast growth factor receptor monomers), thereby inhibiting the activity of the receptor tyrosine kinase. In other words, the moiety may allow ligand induced dimerization of the receptor tyrosine kinase ectodomains, but affect the positioning of the two ectodomains at the cell surface interface or alter or prevent conformational changes in the receptor tyrosine kinases, thereby inhibiting the activity of the receptor tyrosine kinase (e.g., inhibiting receptor internalization and/or inhibiting tyrosine autophosphorylation of the receptor and/or inhibiting the ability of the receptor to activate a downstream signaling pathway).

Thus, in some embodiments, it is useful to employ assays which are able to identify moieties that allow receptor dimerization, yet render the receptor inactive. Such assays are described herein. For example, experiments may be performed with the receptor whereby receptor dimerization is detected using cross linking, and receptor activation is determined using phosphotyrosine specific antibodies.

The conformational state of the RTK may also be determined by Fluorescence Resonance Energy Transfer (FRET) analysis. A comprehensive review of fluorescence methodologies for determining protein conformations and interactions can be found in Johnson (2005) Traffic. 2005 December; 6(12):1078-92 which is incorporated herein by reference. In the FRET assay a RTK of interest is labeled with appropriate FRET fluorophores. After the RTK is labeled, cells expressing the labeled RTK are incubated with test moieties of the invention and the ligand of the RTK (e.g., SCF for the Kit RTK). FRET analysis will allow the observation of conformational changes in the RTK associated with ligand binding, RTK dimerization, and/or receptor activation. By this method one of skill in the art may directly assess a protein conformational change which indicates RTK dimerization without downstream activation. There are a number of methods available to perform FRET analysis, and a large portion of the variation arises from the use of different fluorophores or different techniques to incorporate those fluorophores into proteins of interest. FRET fluorophores and analysis methods are well known in the art, and a brief review of FRET technology is available in Heyduk (2002) Current Opinion in Biotechnology, 13(4):292-296 and references therein. The following publications expand on the FRET method and are incorporated herein by reference: Kajihara et al. (2006) Nat. Methods. 3(11):923-9; Biener-Ramanujan et al. (2006) Growth Horm IGF Res. 16(4):247-57; Taniguchi et al. (2007) Biochemistry. 46(18):5349-57; U.S. Pat. Nos. 6,689,574; 5,891,646; and WIPO Publication No. WO/2002/033102. FRET fluorophores may be incorporated into any domain or hinge region of a RTK to detect conformational changes (e.g., the asymmetric contact interface of an RTK) provided that the fluorophores do not interfere with the function of the RTK or the ability of moieties of the invention to bind the RTK.

Fluorophores useful for FRET are often the same as those useful for Bioluminescence Resonance Energy Transfer (BRET) as discussed below. The most popular FRET method is to engineer reactive cystein residues into a protein of interest. Fluorophores can then easily react with the chosen cystein residues. Often fusion proteins are constructed, whereby a protein of interest is fused to Green Fluorescent Protein (see Neininger et al. (2001) EMBO Reports. 2(8):703-708). Additional methods and useful fluorophores for FRET are described in Huebsch and Mooney (2007) Biomaterials. 28(15):2424-37; Schmid and Birbach (2007) Thromb Haemost. 97(3):378-84; Jares-Erijman AND Jovin (2006) Curr Opin Chem. Biol. 10(5):409-16; Johansson (2006) Methods Mol. Biol. 335:17-29; Wallrabe and Periasamy (2005) Curr Opin Biotechnol. 16(1):19-27; and Clegg R M (1995) Curr Opin Biotechnol. 6(1):103-10 which are incorporated herein by reference.

In other embodiments, it may be unknown or difficult to determine (depending on the receptor) which RTK conformation is specifically indicative of dimerization without ligand-induced trans autophosphorylation. In such cases, one of skill in the art may combine assays that determine receptor dimerization with those that determine receptor activation or trans autophosphorylation. For example, one may use traditional cross-linking studies (exemplified by Rodriguez et al. (1990) Molecular Endocrinology, 4(12), 1782-1790) to detect RTK dimerization in combination with any of the receptor activation assays discussed above. FRET and similar systems may also be used to directly measure receptor activation or dimerization. For example, by incorporating appropriate FRET fluorophores into the cytoplasmic domain of the RTK and into a phosphorylation target protein (i.e., a downstream signaling molecule), FRET would be capable of determining whether downstream signaling molecules were being recruited to the RTK. Therefore, in one embodiment a successful moiety of the invention is one which allows receptor dimerization, as measured by cross-linking or FRET, but which prevents receptor activation, detected as lack of fluorescence by FRET or BRET analysis or by other receptor activation assays (e.g., autophosphorylation assay employing anti-phosphotyrosine antibodies and Western Blot). Thus, using the techniques described herein, one of skill in the art can easily test moieties (e.g., small molecules, peptides, or antibodies) to determine whether they inhibit RTK activity and whether they allow receptor dimerization.

In particular, Bioluminescence Resonance Energy Transfer (BRET) analysis may be used to identify moieties which inhibit the activity of RTKs. U.S. Pat. Pub. No. 20060199226, WIPO Publication No. WO/2006/094073, and Tan et al. (2007. Molecular Pharmacology. 72:1440-1446) specifically describe methods to identify ligands which activate RTKs and are thus incorporated herein by reference. These techniques have been employed for determining protein interactions in vitro and in vivo (Pfleger et al. (2006) Nature Protocols 1 337-345; Kroeger et al. (2001), J. Biol. Chem., 276(16):12736-43; and Harikumar, et al. (2004) Mol Pharmacol 65:28-35; which are all incorporated herein by reference).

BRET is useful for identifying moieties of the present invention from test compounds by screening for those moieties which prevent RTK activation.

As discussed in U.S. Pat. Publication No. 2006/0199226 which is incorporated herein by reference, BRET based assays can be used to monitor the interaction of proteins having a bioluminescent donor molecule (DM) with proteins having a fluorescent acceptor moiety (AM). Briefly, cells expressing an RTK-DM fusion will convert the substrate's chemical energy into light. If there is an AM (e.g., a signaling protein-AM fusion) in close proximity to the RTK-DM fusion, then the cells will emit light at a certain wavelength. For example, BRET based assays can be used to assess the interaction between a RTK-luciferase fusion and a GFP-signalling protein fusion. This differs slightly from FRET analysis, where the donor molecule may be excited by light of a specific wavelength rather than by chemical energy conversion. Examples of bioluminescent proteins with luciferase activity that may be used in a BRET analysis may be found in U.S. Pat. Nos. 5,229,285, 5,219,737, 5,843,746, 5,196,524, 5,670,356. Alternative DMs include enzymes, which can act on suitable substrates to generate a luminescent signal. Specific examples of such enzymes are beta-galactosidase, alkaline phosphatase, beta-glucuronidase and beta-glucosidase. Synthetic luminescent substrates for these enzymes are well known in the art and are commercially available from companies, such as Tropix Inc. (Bedford, Mass., USA). DMs can also be isolated or engineered from insects (U.S. Pat. No. 5,670,356).

Depending on the substrate, DMs emit light at different wavelengths. Non-limiting examples of substrates for DMs include coelenterazine, benzothiazole, luciferin, enol formate, terpene, and aldehyde, and the like. The DM moiety can be fused to either the amino terminal or carboxyl terminal portion of the RTK protein. Preferably, the positioning of the BDM domain within the RTK-DM fusion does not alter the activity of the native protein or the binding of moieties of the present invention. RTK-DM fusion proteins can be tested to ensure that it retains biochemical properties, such as ligand binding and ability to interact with downstream signaling molecules of the native protein.

AMs in BRET analysis may re-emit the transferred energy as fluorescence. Examples of AMs include Green Fluorescent Protein (GFP), or isoforms and derivatives thereof such as YFP, EGFP, EYFP and the like (R. Y. Tsien, (1998) Ann. Rev. Biochem. 63:509-544). Preferably, the positioning of the AM domain within the AM-protein fusion does not alter the activity of the native protein. AM-second protein fusion proteins can be tested to ensure that it retains biochemical properties of the cognate native protein, such as interaction with RTKs. By way of example, an amino terminal fusion of the GFP protein to any substrate which is phosphorylated by or can bind to the target RTK can be used.

### V. Pharmaceutical Compositions Containing the Moieties of the Invention

In another aspect, the present invention provides a composition, e.g., a pharmaceutical composition, containing one or a combination of the moieties of the invention (e.g., monoclonal antibodies, or antigen-binding portion(s) thereof, antibody mimetics, small molecules, or peptidic molecules of the present invention), formulated together with a pharmaceutically acceptable carrier. Such compositions may include one or a combination of (e.g., two or more different) antibodies, or immunoconjugates, small molecules, or peptidic molecules of the invention. For example, a pharmaceutical composition of the invention can comprise a combination of antibodies and small molecules that bind to different epitopes on the target RTK or that have complementary activities, e.g., a small molecule that binds to an asymmetric contact interface of FGFR1 together with a small molecule that binds to an asymmetric contact interface of FGFR2.

Pharmaceutical compositions of the invention also can be administered in combination therapy, i.e., combined with other agents. For example, the combination therapy can include an anti-RTK antibody (or small molecule or peptidic molecule) of the present invention combined with at least one other anti-cancer agent. Examples of therapeutic agents that can be used in a combination therapy are described in greater detail below.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., the moiety of the invention, may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody, small molecule, or peptidic molecule, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for a moiety of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

Alternatively, the antibody, small molecule, or peptidic molecule can be administered as a sustained release formulation, in which case less frequent administration is required. Dosage and frequency vary depending on the half-life of the administered substance in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients and small molecules in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-RTK moiety of the invention preferably results in a decrease in severity of disease symptoms, an increase in frequency and duration of disease symptom-free periods, or a prevention of impairment or disability due to the disease affliction. For example, for the treatment of tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

An anti-RTK moiety of the present invention may be tested to determine whether it is effective in antagonizing the ligand-induced trans autophosphorylation of the RTK. One method of testing the anti-RTK moiety is to confirm that interaction occurs between the anti-RTK moiety and the RTK. For example, one of skill in the art may test whether an antibody, small molecule, or peptidic molecule of the invention binds to an asymmetric contact interface of the RTK. Such tests for binding are well known in the art and may include labeling (e.g., radiolabeling) the anti-RTK moiety, incubating the anti-RTK moiety with an RTK under conditions in which binding may occur, and then isolating/visualizing the complex on a gel or phosphor screen. Similarily, the ELISA technique may be employed to determine binding.

Another method to determine whether the moiety of the invention is antagonizing a RTK is to test the phosphorylation state of the cytoplasmic domain of the RTK. In specific embodiments, effective antagonists will prevent activation and trans autophosphorylation of a RTK. Phosphorylation of the RTK may be tested using standard methods known in the art, for example, by using antibodies which specifically bind the phosphorylated residues of the RTK. Other methods to detect phosphorylation events include those described in U.S. Pat. No. 6,548,266; or Goshe et al. (2006) Brief Funct Genomic Proteomic. 4:363-76; de Graauw et al. (2006) Electrophoresis. 27:2676-86; Schmidt et al. (2007) J Chromatogr B Analyt Technol Biomed Life Sci. 849:154-62; or by the use of the FlashPlates (SMP200) protocol for the Kinase Phosphorylation Assay using [gamma-33P]ATP by PerkinElmer. One of skill in the art will appreciate that these methods, and those demonstrated in the Examples may also be used to determine the phosphorylation state of proteins which are phosphorylated by the RTK and are signal transducers within the cell. Detecting the phosphorylation state of such proteins will also indicate whether the RTK has been effectively antagonized by the moieties of the present invention.

A composition of the present invention can be administered via one or more routes of administration using one or more of a variety of methods known in the art. As will be appreciated by the skilled artisan, the route and/or mode of administration will vary depending upon the desired results. Preferred routes of administration for binding moieties of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an anti-RTK binding moiety of the invention can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition of the invention can be administered with a needleless hypodermic injection device, such as the devices disclosed in U.S. Pat. No. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

### VI. Methods for Using the Moieties of the Invention

In another aspect, the present invention provides a method for treating a RTK associated disease in a subject, comprising administering to the subject a therapeutically effective amount of a moiety of the invention. The anti-RTK moieties, e.g., small molecules, antibodies, or peptidic molecules, of the present invention have numerous in vitro and in vivo diagnostic and therapeutic utilities involving the diagnosis and treatment of a receptor tyrosine kinase associated disease. The binding moieties of the present invention can be administered to cells in culture, in vitro or ex vivo, or to human subjects, e.g., in vivo, to treat, prevent and to diagnose a receptor tyrosine kinase associated disease.

As used herein “a receptor tyrosine kinase associated disease” is a disease or condition which is mediated by RTK activity or is associated with aberrant RTK expression or activation. Examples of receptor tyrosine kinase associated diseases include diseases or conditions that are associated with, for example, FGF receptors, HGF receptors, insulin receptors, IGF-1 receptors, NGF receptors, VEGF receptors, PDGF-receptor-α, PDGF-receptor-β, CSF-1-receptor, and Flt3-receptors, such as age-related macular degeneration (AMD), atherosclerosis, rheumatoid arthritis, diabetic retinopathy or pain associated diseases. Another example of receptor tyrosine kinase associate diseases includes severe bone disorders. Severe bone disorders include disorders selected from the group consisting of achondroplasia, Crouzon syndrome and Saethre-Chotzen syndrome. Specific examples of receptor tyrosine kinase associated diseases include, but are not limited to, gastrointestinal stromal tumors (GIST), acute myelogenous leukemia (AML), small cell lung cancer (SCLC), breast cancer, bone metastatic breast cancer, lymphatic diseases and tenosynovial giant cell tumors. Additional examples of receptor tyrosine kinase associated diseases include glioblastoma, LADD syndrome, achondroplasia, Crouzon syndrome, Saethre-Chotzen syndrome, Antley-Bixler syndrome, hypogonadotropic hypogonadism, Jackson-Weiss syndrome, Kallman syndrome 2, osteoglophonic dysplasia, Pfeiffer syndrome, trigonocephaly, colon cancer (including small intestine cancer), lung cancer, breast cancer, pancreatic cancer, melanoma (e.g., metastatic malignant melanoma), acute myeloid leukemia, kidney cancer, bladder cancer, ovarian cancer and prostate cancer. Examples of other cancers that may be treated using the methods of the invention include renal cancer (e.g., renal cell carcinoma), glioblastoma, lymphatic cancer, brain tumors, chronic or acute leukemias including acute lymphocytic leukemia (ALL), adult T-cell leukemia (T-ALL), chronic myeloid leukemia, acute lymphoblastic leukemia, chronic lymphocytic leukemia, lymphomas (e.g., Hodgkin's and non-Hodgkin's lymphoma, lymphocytic lymphoma, primary CNS lymphoma, T-cell lymphoma, Burkitt's lymphoma, anaplastic large-cell lymphomas (ALCL), cutaneous T-cell lymphomas, nodular small cleaved-cell lymphomas, peripheral T-cell lymphomas, Lennert's lymphomas, immunoblastic lymphomas, T-cell leukemia/lymphomas (ATLL), entroblastic/centrocytic (cb/cc) follicular lymphomas cancers, diffuse large cell lymphomas of B lineage, angioimmunoblastic lymphadenopathy (AILD)-like T cell lymphoma and HIV associated body cavity based lymphomas), embryonal carcinomas, undifferentiated carcinomas of the rhino-pharynx (e.g., Schmincke's tumor), Castleman's disease, Kaposi's Sarcoma, multiple myeloma, Waldenstrom's macroglobulinemia and other B-cell lymphomas, nasopharangeal carcinomas, bone cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, glioblastoma, multiple myeloma, prostate cancer, pancreatic cancer, bladder cancer, breast cancer, solid tumors of childhood, cancer of the bladder, cancer of the kidney or ureter, carcinoma of the renal pelvis, neoplasm of the central nervous system (CNS), tumor angiogenesis, spinal axis tumor, brain stem glioma, pituitary adenoma, epidermoid cancer, squamous cell cancer, environmentally induced cancers including those induced by asbestos, e.g., mesothelioma and combinations of said cancers. Examples of lymphatic diseases, or “diseases of the lymphatic system”, that may be treated using the methods of the invention include afibrinogenemia, anemia, aplastic anemia, hemolytic anemia, congenital nonspherocytic anemia, megaloblastic anemia, pernicious anemia, sickle cell anemia, renal anemia, angiolymphoid hyperplasia with eosinophilia, antithrombin III deficiency, Bernard-Soulier syndrome, blood coagulation disorders, blood platelet disorders, blue rubber bleb nevus syndrome, Chediak-Higashi syndrome, cryoglobulinemia, disseminated intravascular coagulation, eosinophilia, Erdheim-Chester disease, erythroblastosis, fetal, evans syndrome, factor V deficiency, factor VII deficiency, factor X deficiency, factor XI deficiency, factor XII deficiency, fanconi anemia, giant lymph node hyperplasia, hematologic diseases, hemoglobinopathies, hemoglobinuria, paroxysmal, hemophilia a, hemophilia b, hemorrhagic disease of newborn, histiocytosis, histiocytosis, langerhans-cell, histiocytosis, non-langerhans-cell, job's syndrome, leukopenia, lymphadenitis, lymphangioleiomyomatosis, lymphedema, methemoglobinemia, myelodysplastic syndromes, myelofibrosis, myeloid metaplasia, myeloproliferative disorders, neutropenia, paraproteinemias, platelet storage pool deficiency, polycythemia vera, protein c deficiency, protein s deficiency, purpura, thrombocytopenic, purpura, thrombotic thrombocytopenic, RH-isoimmunization, sarcoidosis, sarcoidosis, spherocytosis, splenic rupture, thalassemia, thrombasthenia, thrombocytopenia, Waldenstrom macroglobulinemia, or Von Willebrand disease.

Furthermore, given the expression of type IV RTKs on various tumor cells, the binding moieties, compositions, and methods of the present invention can be used to treat a subject with a tumorigenic disorder, e.g., a disorder characterized by the presence of tumor cells expressing an RTK including, for example, gastrointestinal stromal tumors, mast cell disease, and acute myelogenous leukemia. Examples of other subjects with a tumorigenic disorder include subjects having renal cancer (e.g., renal cell carcinoma), glioblastoma, brain tumors, chronic or acute leukemias including acute lymphocytic leukemia (ALL), adult T-cell leukemia (T-ALL), chronic myeloid leukemia, acute lymphoblastic leukemia, chronic lymphocytic leukemia, lymphomas (e.g., Hodgkin's and non-Hodgkin's lymphoma, lymphocytic lymphoma, primary CNS lymphoma, T-cell lymphoma, Burkitt's lymphoma, anaplastic large-cell lymphomas (ALCL), cutaneous T-cell lymphomas, nodular small cleaved-cell lymphomas, peripheral T-cell lymphomas, Lennert's lymphomas, immunoblastic lymphomas, T-cell leukemia/lymphomas (ATLL), entroblastic/centrocytic (cb/cc) follicular lymphomas cancers, diffuse large cell lymphomas of B lineage, angioimmunoblastic lymphadenopathy (AILD)-like T cell lymphoma and HIV associated body cavity based lymphomas), embryonal carcinomas, undifferentiated carcinomas of the rhino-pharynx (e.g., Schmincke's tumor), Castleman's disease, Kaposi's Sarcoma, multiple myeloma, Waldenstrom's macroglobulinemia and other B-cell lymphomas, nasopharangeal carcinomas, bone cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), tumor angiogenesis, spinal axis tumor, brain stem glioma, pituitary adenoma, epidermoid cancer, squamous cell cancer, environmentally induced cancers including those induced by asbestos, e.g., mesothelioma and combinations of said cancers.

As used herein, the term “subject” is intended to include human and non-human animals. Non-human animals includes all vertebrates, e.g., mammals and non-mammals, such as non-human primates, sheep, dogs, cats, cows, horses, chickens, amphibians, and reptiles. Preferred subjects include human subjects having a receptor tyrosine kinase associated disease.

The moieties (e.g., small molecules, peptidic molecules, antibodies, antigen binding portions thereof, antibody mimetics, and compositions) of the invention have additional utility in therapy and diagnosis of a RTK associated disease. For example, the human monoclonal antibodies, the multispecific or bispecific molecules, the small molecules, or the peptidic molecules can be used to elicit in vivo or in vitro one or more of the following biological activities: to inhibit the growth of and/or kill a cell expressing a RTK (e.g., a fibroblast growth factor receptor); to mediate phagocytosis or

ADCC of a cell expressing a RTK (e.g., a fibroblast growth factor receptor) in the presence of human effector cells; or to inhibit the ligand-induced trans autophosphorylation of the RTK, thereby antagonizing the activity of the receptor.

Suitable routes of administering the anti-RTK moieties of the invention in vivo and in vitro are well known in the art and can be selected by those of ordinary skill For example, the anti-RTK moieties can be administered by injection (e.g., intravenous or subcutaneous). Suitable dosages of the molecules used will depend on the age and weight of the subject and the concentration and/or formulation of the binding moiety composition.

As previously described, the anti-RTK moieties of the invention can be co-administered with one or other more therapeutic agents, e.g., a cytotoxic agent, a radiotoxic agent or an immunosuppressive agent. The moiety can be linked to the agent or can be administered separate from the agent. In the latter case (separate administration), the binding moiety can be administered before, after or concurrently with the agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil and cyclophosphamide hydroxyurea which, by themselves, are only effective at levels which are toxic or subtoxic to a patient. Cisplatin is intravenously administered as a 100 mg/dose once every four weeks and adriamycin is intravenously administered as a 60-75 mg/ml dose once every 21 days. Co-administration of the anti-RTK binding moieties, of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the binding moiety.

When administering anti-RTK moiety-partner molecule conjugates of the present invention for use in the prophylaxis and/or treatment of diseases related to abnormal cellular proliferation, a circulating concentration of administered compound of about 0.001 μM to 20 μM or about 0.01 μM to 5 μM may be used.

Patient doses for oral administration of the compounds described herein, typically range from about 1 mg/day to about 10,000 mg/day, more typically from about 10 mg/day to about 1,000 mg/day, and most typically from about 50 mg/day to about 500 mg/day. Stated in terms of patient body weight, typical dosages range from about 0.01 to about 150 mg/kg/day, more typically from about 0.1 to about 15 mg/kg/day, and most typically from about 1 to about 10 mg/kg/day, for example 5 mg/kg/day or 3 mg/kg/day.

In at least some embodiments, patient doses that retard or inhibit tumor growth can be 1 μmol/kg/day or less. For example, the patient doses can be 0.9, 0.6, 0.5, 0.45, 0.3, 0.2, 0.15, or 0.1 μmol/kg/day or less (referring to moles of the drug). Preferably, the anti-RTK moiety-drug conjugate retards growth of the tumor when administered in the daily dosage amount over a period of at least five days.

In one embodiment, conjugates of the invention can be used to target compounds (e.g., therapeutic agents, labels, cytotoxins, radiotoxoins immunosuppressants, etc.) to cells which have RTK cell surface receptors by linking such compounds to the anti-RTK binding moiety. For example, an anti-RTK moiety can be conjugated to any of the toxin compounds described in U.S. Pat. Nos. 6,281,354 and 6,548,530, US patent publication Nos. 20030050331, 20030064984, 20030073852 and 20040087497 or published in WO 03/022806, which are hereby incorporated by reference in their entireties. Thus, the invention also provides methods for localizing ex vivo or in vivo cells expressing RTK (e.g., with a detectable label, such as a radioisotope, a fluorescent compound, an enzyme or an enzyme co-factor).

Target-specific effector cells, e.g., effector cells linked to compositions (e.g., antibodies, antigen binding portions thereof, small molecules, or peptidic molecules) of the invention can also be used as therapeutic agents. Effector cells for targeting can be human leukocytes such as macrophages, neutrophils or monocytes. Other cells include eosinophils, natural killer cells and other IgG- or IgA-receptor bearing cells. If desired, effector cells can be obtained from the subject to be treated. The target-specific effector cells can be administered as a suspension of cells in a physiologically acceptable solution. The number of cells administered can be in the order of 10⁸-10⁹ but will vary depending on the therapeutic purpose. In general, the amount will be sufficient to obtain localization at the target cell, e.g., a tumor cell expressing RTK and to effect cell killing by, e.g., phagocytosis. Routes of administration can also vary.

Therapy with target-specific effector cells can be performed in conjunction with other techniques for removal of targeted cells. For example, anti-tumor therapy using the moieties of the invention and/or effector cells armed with these compositions can be used in conjunction with chemotherapy.

The invention further provides methods for detecting the presence of a human RTK antigen in a sample, or measuring the amount of human RTK antigen (e.g., an asymmetric contact interface of a human fibroblast growth factor receptor), comprising contacting the sample, and a control sample, with and RTK binding moiety, e.g., a small molecule, peptidic molecule, human monoclonal antibody, or other binding moiety, which specifically binds to a human RTK, under conditions that allow for formation of a complex between the antibody or other moiety and a human RTK such as a fibroblast growth factor receptor. The formation of a complex is then detected, wherein a difference complex formation between the sample compared to the control sample is indicative the presence of RTK, e.g., the fibroblast growth factor receptor, in the sample.

Also within the scope of the present invention are kits comprising the anti-RTK binding moieties (e.g., small molecules, antibodies, antigen binding portions thereof, or peptidic molecules) and instructions for use. The kit can further contain one more additional reagents, such as an immunosuppressive reagent, a cytotoxic agent or a radiotoxic agent or one or more additional anti-RTK moieties of the invention (e.g., an anti-RTK binding moiety having a complementary activity which binds to an epitope in the RTK antigen distinct from the first anti-RTK moiety). Kits typically include a label indicating the intended use of the contents of the kit. The term label includes any writing, or recorded material supplied on or with the kit, or which otherwise accompanies the kit.

The present invention is further illustrated by the following examples, which should not be construed as further limiting. The contents of all figures and all references, patents and published patent applications cited throughout this application, as well as the Figures, are expressly incorporated herein by reference in their entirety.

### EXAMPLES

Ligand induced tyrosine trans autophosphorylation plays an important role in the control of activation and cell signaling by receptor tyrosine kinases (RTK) (Schlessinger (1988) Trends Biochem. Sci., 13(11):443-447; Schlessinger (2000) Cell, 103(2):211-225; Schlessinger and Lemmon (2003) Sci. STKE, 191:RE12; Schlessinger and Ullrich (1992) Neuron, 9(3):383-391; Lemmon and Schlessinger (1994) Trends Biochem. Sci., 19(11):459-463; and Lemmon and Schlessinger (1998) Methods Mol. Biol., 84:49-71). Structural and biochemical studies have shown that autophosphorylation of fibroblast growth factor receptor 1 (FGFR1) (Furdui et al. (2006) Mol. Cell., 21(5):711-717 and Lew et al. (2009) Sci. Signal, 2(58):ra6) and FGFR2 (Chen et al. (2008) Proc. Natl. Acad. Sci. U.S.A., 105(50):19660-19665) are mediated by a sequential and precisely ordered intermolecular reaction that can be divided into three phases. The first phase involves trans phosphorylation of a tyrosine located in the activation loop (Y653 in FGFR1) of the catalytic core resulting in 50-100 fold stimulation of kinase activity (Furdui et al., 2006). In the second phase, tyrosine residues that serve as docking sites for signaling proteins are phosphorylated including tyrosines in the kinase insert region (Y583, Y585), the juxtamembrane region (Y463) and in the C-terminal tail (Y766) of FGFR1. In the final and third phase, Y654; a second tyrosine located in the activation loop is phosphorylated, resulting in an additional 10 fold increase in FGFR1 kinase activity (Furdui et al., 2006). Interestingly, tyrosines that are adjacent to one another (e.g., Y653, Y654 and Y583, Y585) are not phosphorylated sequentially, suggesting that both sequence and structural specificities dictate the order of phosphorylation. Although tyrosine phosphorylation plays a major role in cell signaling, it is not yet clear what the structural basis for trans autophosphorylation is. In other words, the molecular mechanism underlying how one kinase (the enzyme) within the dimerized receptor specifically and sequentially catalyzes phosphorylation of tyrosine(s) of the other kinase (the substrate) has not yet been resolved.

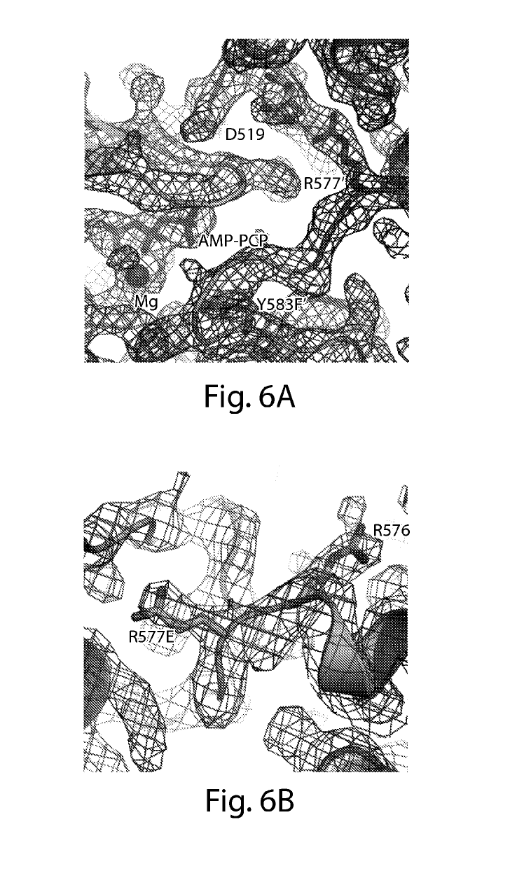

The crystal structure of activated FGFR1 kinase domain bound to a phospholipase Cγ (PLCγ fragment composed of two SH2 domains and a tyrosine phosphorylation site has been demonstrated (PDB code 3GQI) (Bae et al. (2009) Cell 138(3):512-524). In this structure, the substrate-binding pocket of the kinase molecule (the enzyme molecule, termed molecule E) is occupied by Y583F of a symmetry-related molecule (the substrate molecule, termed molecule S). This tyrosine (Y583F) is located in the kinase insert and is the second FGFR1 tyrosine that becomes phosphorylated in vitro (Furdui et al., 2006). On closer examination of the crystal structure, 3GQI, a substantial crystallographic interface was identified between the N-lobe of the molecule that serves as an enzyme and the C-lobe of molecule that functions as a substrate. In this interface there are direct interactions between R577′ and D519 (FIG. 6A). Inherited mutations have been documented that result in D519N, a loss of function mutation causing LADD syndrome (Rohmann et al. (2006) Nat. Genet. 38(4):414-417), and in R576W, a somatic gain of function mutation found in glioblastoma (Rand et al. (2005) Proc. Natl. Acad. Sci. U.S.A., 102(40):14344-14349). Structural and biochemical tools were utilized to show that R577 is involved in creating, in vivo, an asymmetric FGFR1 dimer that allows trans phosphorylation of Y583 and other tyrosine autophosphorylation sites in FGF stimulated cells. These data provide the basis for understanding molecular-level specificity in FGFR1 trans phosphorylation and cell signaling.

### Example 1

### Asymmetric Dimerization Interface During Autophosphorylation of FGFR1

The structure of activated FGFR1 kinase in complex with a phospholipase Cγ (PLCγ) fragment (Bae et al. (2009) Cell, 138(3):512-524) shows that two symmetry-related activated kinase domains form an asymmetric dimer which illustrates in vivo trans-autophosphorylation of Y583 in the kinase insert region (FIG. 1A and FIG. 1B). The asymmetric arrangement of the two kinase molecules is mediated by an interface formed between the activation segment, the tip of nucleotide-binding loop, the β3-αC loop, the β4-β5 loop and the N-terminal region of helix αC in a kinase molecule that serves as an enzyme (E), and the kinase insert and residues between C-lobe helices αF and αG in a second kinase molecule serving as a substrate (S) (FIG. 1C and FIG. 1D). Importantly, R577, a residue close to the kinase insert region of the substrate molecule, contributes to this interface (FIG. 1B). The interface buries approximately 800 Å² (Laskowski et al. (1997) Trends Biochem. Sci., 22(12):488-490).

The interface formed between the two active FGFR1 molecules consists of two regions. One is the proximal substrate-binding site near the P+1 region of the activation segment. The other is a region distal from the substrate-binding site. The distal substrate-binding site is formed between a region adjacent to the nucleotide-binding loop of molecule E and the αF-αG loop and the N-terminal residues of the kinase insert region of molecule S. In the crystal structure clear electron density is seen for R577 and D519 (FIG. 6A). It is of note that the R577 side chain faces approximately 180° opposite from that of R576; an amino acid mutated in glioblastoma (Rand et al. (2005) Proc. Natl. Acad. Sci. U.S.A., 102(40):14344-14349).

The two regions of the asymmetric dimer interface are complementary (FIG. 2). For the proximal substrate-binding site molecule E predominantly contributes residues from the activation segment (N659-V664) that form a short antiparallel 13-sheet with residues C-terminal to Y583′ from molecule S, and R570 forms a salt bridge with E582′ (FIG. 2B). For the distal binding site, R577′ binds both the backbone carbonyl and side chain of D519, and the loop between helices αF and αG in molecule S forms multiple aliphatic contacts with the β3-αC and β4-β5 loops (FIG. 2C).

### Example 2

### In Vitro Tyrosine Kinase Activity of the R577E FGFR1 Mutant

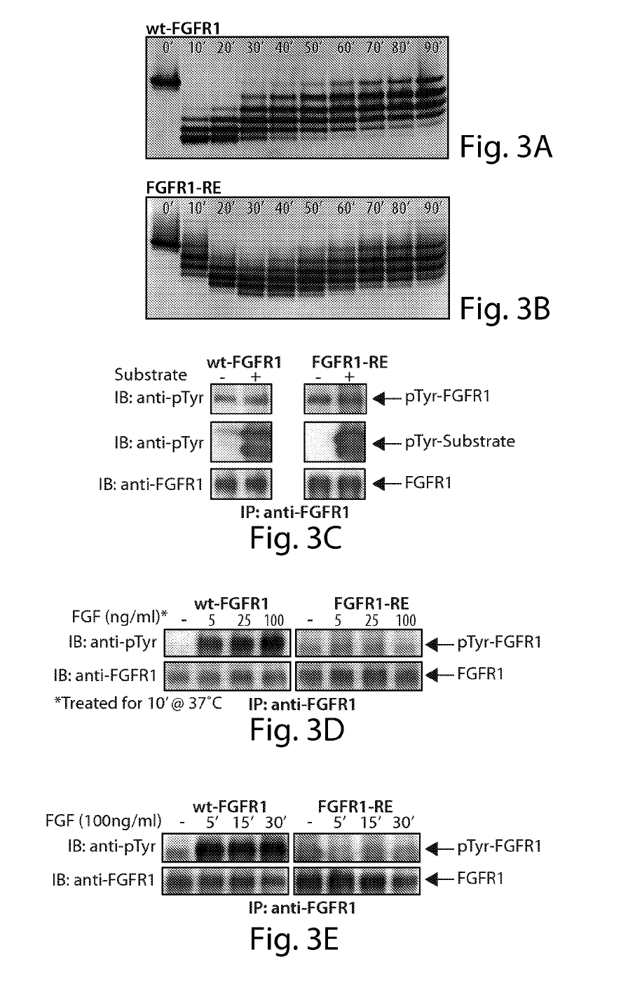

To investigate the in vitro effects of R577E mutation (FGFR1-RE) autophosphorylation experiments using wt-FGFR1 and FGFR1-RE kinase domains were conducted. Purified FGFR1 kinase domains were incubated with ATP and Mg²⁺ at room temperature and monitored at different times by stopping the trans phosphorylation reaction with EDTA and running all samples on a non-reducing native gel (FIG. 3A and FIG. 3B). The reaction profiles of wt-FGFR1 and FGFR1-RE in native gels clearly showed that trans phosphorylation and the reverse dephosphorylation reaction of FGFR1-RE were substantially retarded when compared to those of wt-FGFR1 kinase domain. Trans phosphorylation of wt-FGFR1 kinase domain took place within 10 minutes, reaching a fully phosphorylated state, and then underwent the reverse dephosphorylation reaction. This contrasts with FGFR1-RE, which became fully phosphorylated within 30 minutes and then underwent the reverse reaction. This experiment demonstrates that the intrinsic kinase activity of FGFR1-RE kinase domain is maintained; yet it is kinetically retarded.

To study how the R577E mutation affects the activity and trans phosphorylation of full length FGFR1, wt-FGFR1 and FGFR1-RE were stably expressed in L6 myoblasts (FIG. 3C). Lysates from cells expressing wt-FGFR1 or FGFR1-RE were immunoprecipitated and subjected to an in vitro autophosphorylation reaction at room temperature (Furdui et al. (2006) Mol. Cell., 21(5):711-717). FIG. 3C shows that both full length wt-FGFR1 and FGFR1-RE become tyrosine autophosphorylated to a similar extent and are capable of phosphorylating an exogenous substrate molecule composed of the two SH2 domains and a phosphorylation site of PLCγ. These results demonstrate that the tyrosine kinase activity of full length R577E FGFR1 mutant is maintained in vitro.

### Example 3

### Tyrosine Autophosphorylation of the R577E Mutant is Strongly Compromised in Living Cells

Autophosphorylation of WT or the R577E FGFR1 mutant in FGF stimulated live cells were compared. Stable L6 cell lines matched for expression level of wt-FGFR1 or FGFR1-RE were stimulated with different FGF concentrations for 10 minutes at 37° C. (FIG. 3D) or with 100 ng/ml FGF at different time points (FIG. 3E). The level of receptor tyrosine phosphorylation was determined by subjecting lysates from unstimulated or FGF stimulated cells to immunoprecipitation with anti-FGFR1 antibodies followed by immunoblotting with anti-pTyr antibodies. FGF stimulation of cells expressing wt-FGFR1 resulted in ligand-dependent receptor tyrosine phosphorylation. By contrast, FGF stimulation of cells expressing FGFR1-RE resulted in a very weak phosphorylation even at the highest dose of the ligand. The drastic reduction in tyrosine autophosphorylation of FGFR1-RE in vivo, is not caused by the loss of its intrinsic kinase activity since both isolated full-length R577E mutant and the purified kinase domain of the R577E mutant maintained kinase activity in vitro.

### Example 4

### Crystal Structure of R577E Mutant

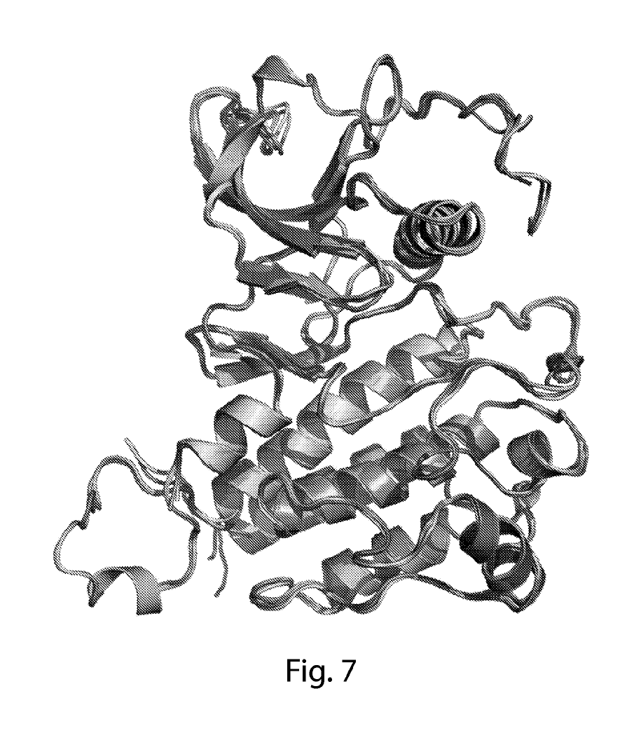

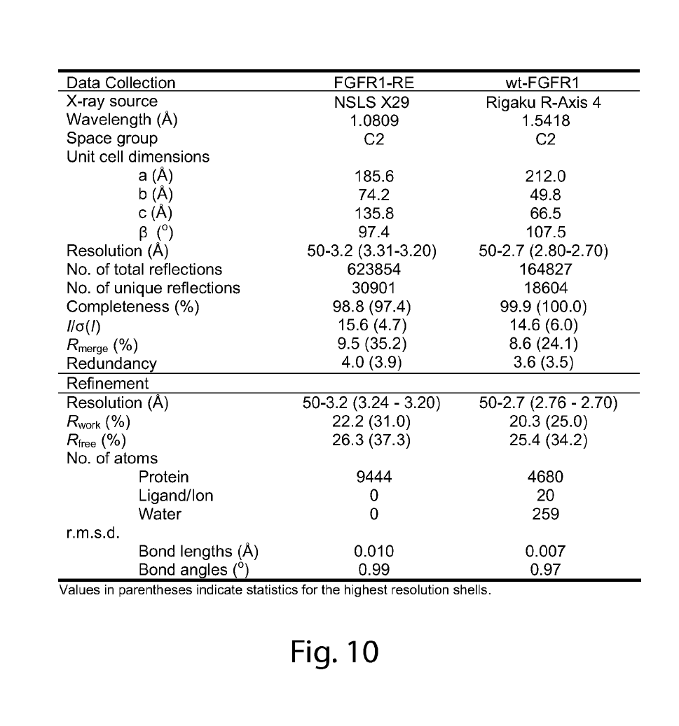

The effect of the R577E mutation on the integrity of the kinase domain was examined by determining the crystal structure of the kinase domain of an FGFR1 mutant protein. The R577E mutant protein was expressed in E. coli and purified by affinity, size exclusion and anion exchange chromatography. Rod-shaped crystals of mutant protein grew in 2 weeks at room temperature and diffract to 3.2 Å resolution. These crystals belong to space group C2 and include four copies of FGFR1-RE in the asymmetric unit. All four molecules of FGFR1-RE are in very similar conformations and superpose with RMSDs less than 0.4 Å over residues 461 to 762 without the kinase insert region (aa. 576-594) (FIG. 7) (www.pymol.org); the kinase insert is flexible and modeled in only two of the four molecules. All four FGFR1-RE molecules are in the active state and exhibit few changes in overall conformation when compared to 3GQI. The structure of unphosphorylated FGFR1 was also determined in the inactive conformation to 2.70 Å resolution (Table 1; FIG. 10).

Briefly, the FGFR1-RE mutant crystal structure is an active-state kinase domain with an extended activation loop. The N-lobe is rotated towards the C-lobe of the kinase structure as has previously been seen in activated phosphorylated FGFR structures (Chen et al. (2008) Proc. Natl. Acad. Sci. U.S.A., 105(50):19660-19665 and Bae et al. (2009) Cell, 138(3):514-524). In the structure of FGFR1-RE, no density for ATP analog, ACP-PCP, was found in the catalytic cleft between the N-lobe and C-lobe. Helix αC in the N-lobe of FGFR1-RE is rotated slightly closer to the activation loop than in previously determined structures of FGFR1 (FIG. 4A and FIG. 4B and FIG. 4C). The two unphosphorylated activation loop tyrosines (Y653, Y654) in FGFR1-RE are located in the same positions as phosphotyrosines (pY653, pY654) in the active FGFR1 structure.

### Example 5

### Comparison of FGFR1 Crystal Structures

The conformation of the FGFR1 kinase insert region shows significant conformational flexibility between all three FGFR1 structures (FGFR1 and FGFR1-RE determined in this study and 3GQI, termed FGFR1-3P), with RMSD between 4 and 5.4 Å over residues 576 to 594) (www.pymol.org) (FIG. 4). For FGFR1-RE the asymmetric dimer discussed above, where one kinase domain presents itself as a substrate for a partner kinase domain, is not seen; in FGFR1-RE none of the four molecules in the asymmetric unit does the kinase insert region present itself to the catalytic cleft of another FGFR1 molecule. In all three structures, R576 maintains a similar orientation. However, in the FGFR1-RE structure the orientation of side-chain R577E is flipped approximately 180° compared to wild-type FGFR1 (FIG. 4D). This crystallographically-seen alteration in the conformation of residue 577 illustrates a change in structural space that this loop samples over time.

Upon receptor activation and initiation of trans phosphorylation there is a specific sequence of tyrosine phosphorylation that ensues. This means that dimerization surfaces between two kinase domains are sequentially utilized to allow phosphorylation in the correct order and implies that specific interactions between the two kinase molecules will play important roles in phosphorylation of each specific tyrosine. This is surprising, as the same enzymatic reaction occurs at each phosphorylation site within the protein, suggesting similar surface properties of the intermolecular interaction.

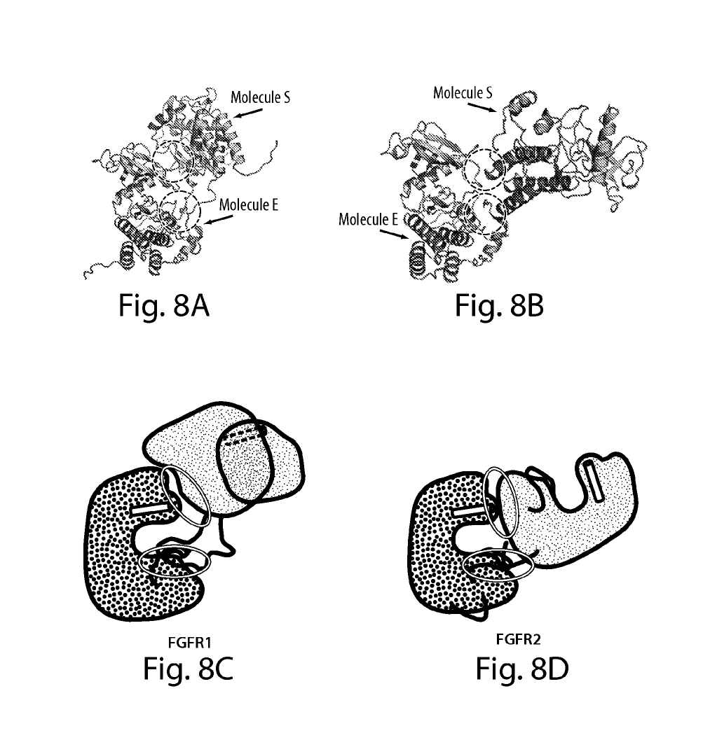

Recently, an asymmetric dimer was described for the kinase domain of FGFR2 (Chen et al. (2008) Proc. Natl. Acad. Sci. U.S.A., 105(50):19660-19665). In the FGFR2 crystal structure Y769 (equivalent to Y766 in FGFR1) is trapped in a position that seems poised to be a substrate for the other kinase domain. Y769 is located at the extreme C-terminus of the kinase domain. Comparison of these two structures (PDB IDs: 3CLY and 3GQI) vividly illustrates the relationship between two FGFR family kinase domains that act as either the enzyme (molecule E) or the substrate (molecule S) (FIG. 8). In both structures the buried surface area of the interface is in the range of 800-900 Å² (Laskowski et al. (1997) Trends Biochem. Sci., 22(12):488-490) and is comprised of a proximal and a distal binding site. The two structures show that at the proximal substrate-binding region there is high structural similarity in the C-lobe of molecule E. However, the distal substrate-binding region is significantly different between the two structures. The N-lobe residues of molecule E that comprise the distal substrate-binding surface are conformationally divergent (FIG. 2 and FIG. 8). In the structure of FGFR1 the distal binding site is formed by residues in the β3-αC loop and by amino acids from the nucleotide-binding loop that contributes only A488 and F489 to the interaction. However, although the conformation of the β3-αC loop is largely unchanged from FGFR1, the nucleotide-binding loop of FGFR2 is significantly altered in conformation and all residues from G488 to G493 contribute to binding. Therefore, structural differences in the kinase N-lobe alter the distal substrate-binding site and are likely important for the sequential nature of trans autophosphorylation.

Structure-based sequence alignment of FGFRs shows conservation of residues involved in the formation of interfaces found in structures of both active FGFR1 and FGFR2 (FIG. 9A) (Notredame et al. (2000): J. Mol. Biol., 302(1):205-217). Interestingly, the N-terminal tip of the helix αG and the adjacent region in the N-terminal loop of the helix αG in FGFR1 is involved in interface formation as a part of the substrate (molecule S) whereas the same region in FGFR2 structure is part of the enzyme (molecule E). In addition, the loop C-terminal to the helix αG is involved in interface formation as a part of the substrate in the FGFR2 structure. On both loops connecting the helix αG to the main body of the kinase several loss-of-function mutations have been clinically discovered (FIG. 9B) (Wilkie (2005) Cytokine Growth Factor Rev., 16(2):187-203). The loss of autophosphorylation activity of the receptors in vivo may come from the disruption of interface formation for trans phosphorylation.

### Conclusions for Examples 1-5

Receptor tyrosine kinases trans phosphorylate in response to ligand activation in a specific sequence, however, the molecular mechanism responsible for this sequential order of trans phosphorylation events is not understood. For FGFR1, there are two regions that mediate asymmetric dimer formation when Y583 is trans phosphorylated. Furthermore, the single point mutation of a residue intrinsic to this interface, R577E, drastically reduces autophosphorylation of FGFR1-RE in live cells. To confirm that this mutation did not alter the kinase fold and did not introduce significant conformational changes, FGFR1-RE was crystallized and no significant differences to wt-FGFR1 were identified (FIG. 4B and FIG. 4C). This confirms that the loss of ligand induced FGFR1 autophosphorylation in living cells is not caused by a conformational change in the kinase domain. The drastic reduction of autophosphorylation of FGFR1-RE mutant in live cells can be addressed by the steric constraints driven by the ligand-induced dimerization in vivo. In living cells autophosphorylation is mediated by FGF and heparan sulfate proteoglycan induced FGFR1 dimerization. Under these conditions interactions among kinase domains in two dimensions increase the steric constraints and decrease the probability of positions between kinase domains within a dimeric complex. That is, only a limited number of modes of interaction between kinase domains of receptor molecules in the cytoplasmic face of the cell membrane are allowed. In an in vitro environment on the other hand, the kinase domains is not subjected to steric constraints generated by receptor dimerization in the cell membrane allowing for freedom to move in three dimension enabling trans phosphorylation of Y583 and other tyrosine residues. However, ligand-induced dimeric FGFR1-RE in vivo cannot bypass the disrupted interface due to the steric constraints generated by dimerization resulting in the failure of trans phosphorylation.

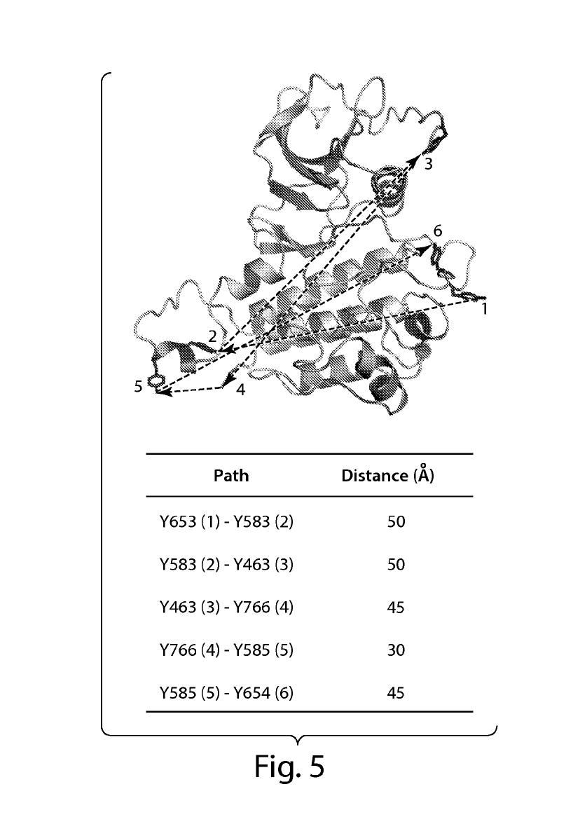

Trans phosphorylation of FGFR1 occurs in precisely ordered sequence (Furdui et al. (2006) and Lew et al. (2009)) and the phosphorylation of all tyrosine auto-phosphotyrosine sites is required for the full FGFR1 activation (Mohammadi et al. (1996) Mol. Cell. Biol., 16(3):977-989). The order of trans phosphorylation sites of FGFR1 is as follows: Y653, Y583, Y463, Y766, Y585, and Y654. Strikingly, the distance between each of these sequential tyrosine phosphorylation sites is between 35-50 Å (FIG. 5). The phosphorylation of Y583 comes second in the order after the phosphorylation of Y653 in the activation loop. Furthermore, the full activation of FGFR1 is achieved by the phosphorylation of Y654 in the activation loop, which is the last residue to be phosphorylated in sequence. The failure of the phosphorylation of Y583 may result in attenuation or termination of trans phosphorylation, resulting in strong inhibition of receptor autophosphorylation in living cells.

### Materials and Methods for Examples 1-5

### Protein Expression and Purification

Site-directed mutagenesis was performed to introduce the mutant (R577E), and transformed into E. coli strain BL21 (codon+). Cultures were grown in terrific broth (TB) media at 37° C. to an OD₆₀₀ of 0.8 and induced with 1 mM isopropyl-thiogalactopyranoside (IPTG) at 18° C. for 10 hours. Cells were harvested and resuspended in lysis buffer (20 mM Tris-HCl pH 8.0, 20 mM NaCl, and 2 mM phenylmethyl-sulphonyl fluoride (PMSF)) then lysed by French press followed by centrifugation to remove cellular debris. Expression and purification of WT and FGFR1 R577E mutant (aa 458-765) were performed as previously described (Furdui et al., 2006). Proteins were first isolated by affinity chromatography on Ni-NTA beads (GE Healthcare) and eluted with an imidazole gradient up to 250 mM. The eluted sample was subsequently subjected to size-exclusion chromatography using Superdex-200 (S200, GE Healthcare), and further purified by Mono-Q (GE Healthcare) ion-exchange chromatography. The purity and mass of the purified protein was verified by electrospray mass spectroscopy.

### Crystallization and structure determination

Proteins were concentrated to 12 mg/ml. The R577E mutant protein was transferred to the buffer containing 1 mM AMP-PCP, 6 mM MgCl₂, 2 mM tris[2-carboxyethyl]phosphine hydrochloride (TCEP-HCl), 20 mM Tris pH 8.0 and 100 mM NaCl, and subjected to screening and optimization. Crystals were grown at room temperature in 14 days using the hanging drop technique containing equal volumes of protein solution and reservoir buffer (15% [w/v] polyethylene glycol 3350, 200 mM lithium citrate). Crystals belonged to the centered monoclinic space group C2 with unit cell dimensions of α=186.8 Å, b=74.3 Å, c=135.8 Å, and β=97.4° with four molecules in the asymmetric unit. The solvent content of the complex was around 61%. Crystals were transferred into the cryoprotectant containing reservoir buffer with 15% glycerol then flash frozen in liquid nitrogen. Crystals of wt-FGFR1 were obtained as described (14). Wt-FGFR1 crystals belonged to C2 space group with unit cell dimensions of a=212.0 Å, b=49.8 Å, c=66.5 Å, and β=107.5°. Data were collected on beamline X29 at the National Synchrotron Light Source for R577E FGFR1 mutant and using the home source for the wild-type protein. Data were processed using HKL2000 (Minor et al. (2000) Structure, 8(5):R105-110). A molecular replacement solution for FGFR1 was found with Phaser (McCoy et al. (2007) J. Appl. Crystallogr., 40(Pt. 4):658-674) using the structures of the kinase domains of FGFR1 (Mohammadi et al. (1996) Cell, 86(4):577-587) (PDB code: 1FRK) and of FGFR-3P (Bae et al. (2009) Cell, 138(3):514-524) (PDB code: 3GQI). Model building and the refinement of wt- and mutant FGFR1 were carried out with Coot (Emsley and Cowtan (2004) Acta Crystallogr. D. Biol. Crystallogr., 60(Pt. 12, Pt. 1):2126-2132) and CNS (Brunger et al. (1998) Acta Crystallogr. D. Biol. Crystallogr., 54(Pt. 5):905-921) to a crystallographic R and R_{free} for FGFR1-RE of 22.2% and 26.3%, and for wt-FGFR1 of 20.3% and 25.4%, respectively. Figures were prepared using PYMOL (www.pymol.org). PDBsum was used to calculate inter molecular interfaces (Laskowski et al. (1997) Trends Biochem. Sci., 22(12):488-490). Data and refinement statistics are summarized in Table 1.

### In Vitro Trans Phosphorylation of wt-FGF1 and FGFR1-RE

1 μl of purified wt-FGFR1 (aa. 458-765) or FGFR1-RE (10 mg/ml) was mixed with 1 μl of each 25 mM ATP, 125 mM MgCl₂ (in 10 mM HEPES pH 7.5) and 10 mM HEPES pH 7.5, then quenched with the 1 μl of 250 mM EDTA in 10 mM HEPES pH 7.5 at every 10 minutes until 90 minutes at room temperature. Native gel electrophoresis was performed with reaction samples with 7% native gels.

### Cell Culture, Immunoprecipitation, and Immunoblotting Experiments

A retroviral vector, pBABE, containing a puromycin resistance gene was utilized for generation of stable cell lines expressing wt-FGFR1 or the R577E FGFR1 mutant in L6 myoblasts. Cells were grown in DMEM containing 10% FBS and penicillin/streptomycin. For experiments, cells were starved overnight in DMEM containing penicillin/streptomycin, and subsequently, stimulated for 10 min with 100 ng/ml FGF. Cell lysates were subjected to immunoprecipitation followed by immunoblotting with various antibodies. Anti-phosphotyrosine (4G10) antibodies were obtained from Upstate Biotechnology, and anti-FGFR1 antibodies were previously described (Furdui et al., 2006 and Lew et al., 2009).

### EQUIVALENTS

Those skilled in the art will recognize, or be able to ascertain using no more that routine experimentation, many equivalents to the specific embodiments of the invention described herein. Such equivalents are intended to be encompassed by the following claims.

1. A moiety that binds to an asymmetric contact interface of a receptor tyrosine kinase (RTK), wherein the moiety inhibits ligand-induced trans autophosphorylation of the RTK.

2. The moiety of claim 1, wherein the moiety does not bind to a nucleotide binding site of a catalytic domain of the RTK.

3. The moiety of claim 1, wherein the moiety binds to an asymmetric contact interface on the N-lobe of one monomer of the RTK.

4. The moiety of claim 1, wherein the moiety binds to an asymmetric contact interface on the C-lobe of one monomer of the RTK.

5. The moiety of claim 1, wherein the moiety does not cause the loss of intrinsic kinase activity.

6. The moiety of claim 1, wherein the moiety increases steric constraints between RTK monomers.

7. The moiety of claim 1, wherein the moiety does not prevent dimerization of the RTK.

8. The moiety of claim 1, wherein the moiety prevents dimerization of the cytoplasmic domains of the RTK.

9. The moiety of claim 1, wherein the RTK is a fibroblast growth factor receptor (FGFR).

10. The moiety of claim 9, wherein the fibroblast growth factor receptor is a) fibroblast growth factor receptor 1 (FGFR1); b) fibroblast growth factor receptor 2 (FGFR2); c) fibroblast growth factor receptor 3 (FGFR3); or d) fibroblast growth factor receptor 4 (FGFR4).

11-13. (canceled)

14. The moiety of claim 1, wherein the moiety binds to amino acid residue Arg577 of FGFR1, Asp519 of FGFR1, Arg579 of FGFR2 or Arg580 of FGFR2; b).

15. (canceled)

16. The moiety of claim 1, wherein the moiety binds to an amino acid residue selected from the group consisting of a) C488, F489, S518, T521, E522, D554, G555, P556, Q574, P587, P579, W691, T695, P702, G703 and P705 of FGFR1; or C491, F492, R577, P582, I590, P705, G706 and P708 of FGFR2.

17. The moiety of claim 1, wherein said moiety binds to at least two amino acid residues selected from the group consisting of a) R577, D519, C488, F489, S518, T521, E522, D554, G555, P556, Q574, P587, P579, W691, T695, P702, G703 and P705 of FGFR1; or b) C491, F492, R577, P582, I590, P705, G706 and P708 of FGFR2.

18-19. (canceled)

20. The moiety of claim 1, wherein said moiety binds to a region of the RTK selected from the group consisting of the β1-β2 loop of a monomer of the RTK, the β3-αC loop of a monomer of the RTK, the β4-B5 loop of a monomer of the RTK, the αD-αE loop of a monomer of the RTK, the αF helix of a monomer of the RTK and the αF-αG loop of a monomer of the RTK.

21. The moiety of claim 1, wherein the moiety binds to a conformational epitope on the RTK.

22. The moiety of claim 21, wherein said conformational epitope is composed of two or more residues in the asymmetric contact interface of the RTK.

23. The moiety of claim 21, wherein said conformational epitope comprises an amino acid residue selected from the group consisting of a) R577, D519, C488, F489, S518, T521, E522, D554, G555, P556, Q574, P587, P579, W691, T695, P702, G703 and P705 of FGFR1; or b) C491, F492, R577, P582, I590, P705, G706 and P708 of FGFR2.

24. (canceled)

25. The moiety of claim 1, wherein the moiety binds to a contiguous epitope on the RTK.

26. The moiety of claim 25, wherein the contiguous epitope is composed of two or more residues in the asymmetric contact interface of the RTK.

27. The moiety of claim 1, wherein the moiety is a small molecule.

28. The moiety of claim 27, wherein the small molecule a) binds to at least one of the amino acid residues selected from the group consisting of amino acid residue R577, D519, C488, F489, S518, T521, E522, D554, G555, P556, Q574, P587, P579, W691, T695, P702, G703 and P705 of FGFR1; b) binds to a region selected from the group consisting of the β1-β2 loop of a monomer of the RTK, the β3-αC loop of a monomer of the RTK, the β4-B5 loop of a monomer of the RTK, the αD-αE loop of a monomer of the RTK, the αF helix of a monomer of the RTK and the αF-αG loop of a monomer of the RTK; or c) is designed based on the asymmetric contact interface of a fibroblast growth factor receptor (FGFR).

29-30. (canceled)

31. The moiety of claim 1, wherein the moiety is a peptidic molecule.

32. The moiety of claim 31, wherein the peptidic molecule is designed based on the asymmetric contact interface of a fibroblast growth factor receptor (FGFR).

33. The moiety of claim 32, wherein the peptidic molecule a) binds to at least one of the amino acid residues selected from the group consisting of amino acid residue R577, D519, C488, F489, S518, T521, E522, D554, G555, P556, Q574, P587, P579, W691, T695, P702, G703 and P705 of FGFR1; b) binds to a region selected from the group consisting of the β1-β2 loop of a monomer of the RTK, the β3-αC loop of a monomer of the RTK, the β4-B5 loop of a monomer of the RTK, the αD-αE loop of a monomer of the RTK, the αF helix of a monomer of the RTK and the αF-αG loop of a monomer of the RTK; c) comprises a structure which is at least 80% identical to amino acid residues 576-594 of FGFR1; or d) comprises a structure which is at least 80% identical to amino acid residues 579-597 of FGFR2.

34-36. (canceled)

37. The moiety of claim 1, wherein the moiety is an isolated antibody, or an antigen-binding portion thereof.

38. The moiety of claim 37, wherein the isolated antibody, or antigen-binding portion thereof, a) is an intrabody; b) is selected from the group consisting of a human antibody, a humanized antibody, a bispecific antibody, and a chimeric antibody; c) is a single chain Fv fragment, an SMIP, an affibody, an avimer, a nanobody, and a single domain antibody; or d) binds to the asymmetric contact interface of a receptor tyrosine kinase with a KD selected from the group consisting of 1×10⁻⁷ M or less, more preferably 5×10⁻⁸ M or less, more preferably 1×10⁻⁸ M or less, more preferably 5×10⁻⁹ M or less.

39. (canceled)

40. The moiety of claim 38, wherein said antibody, or antigen-binding portion thereof, comprises a heavy chain constant region selected from the group consisting of IgG1, IgG2, IgG3, IgG4, IgM, IgA and IgE constant regions.

41-43. (canceled)

44. A hybridoma which produces the antibody, or antigen binding portion thereof, of claim 37.

45. A moiety that binds to a conformational epitope on an asymmetric contact interface of a fibroblast growth factor receptor (FGFR), wherein the moiety inhibits ligand induced trans autophosphorylation of the FGFR.

46. A moiety that binds to an amino acid residue selected from the group consisting of R577, D519, C488, F489, S518, T521, E522, D554, G555, P556, Q574, P587, P579, W691, T695, P702, G703 and P705 of FGFR1, or within 1-5 Å of said residue, thereby inhibiting ligand induced trans autophosphorylation of FGFR1; or b) C491, F492, R577, P582, I590, P705, G706 and P708 of FGFR2.

47. (canceled)

48. A moiety that binds to an asymmetric contact interface of a receptor tyrosine kinase (RTK), wherein a) the moiety disrupts the interface between the N-lobe of an RTK monomer which serves as an enzyme and the C-lobe of an RTK monomer which serves as a substrate; or b) the moiety inhibits reverse dephosphorylation of the RTK.

49. (canceled)

50. A pharmaceutical composition comprising the moiety of any one of claims 1, 45, 46 or 48 and a pharmaceutically acceptable carrier.

51. A method for treating or preventing an RTK associated disease in a subject, the method comprising administering to said subject an effective amount of the moiety of any one of claims 1, 45, 46 or 48, thereby treating or preventing said RTK associated disease in said subject.

52. The method of claim 51, wherein the RTK associated disease is selected from the group consisting of cancer and severe bone disorders.

53. The method of claim 52, wherein the severe bone disorder is a disorder selected from the group consisting of achondroplasia, Crouzon syndrome, and Saethre-Chotzen syndrome; and wherein the cancer is selected from the group consisting of glioblastoma, multiple myeloma, prostate cancer, pancreatic cancer, bladder cancer and breast cancer.

54. (canceled)

55. A method for identifying a moiety that binds to an asymmetric contact interface of a receptor tyrosine kinase (RTK) and inhibits ligand-induced trans autophosphorylation of the RTK, the method comprising:
contacting a RTK with a candidate moiety;
simultaneously or sequentially contacting said RTK with a ligand for the RTK;
determining whether said moiety affects the positioning, orientation and/or distance between the N-lobe of an RTK monomer which functions as an enzyme and the C-lobe of an RTK monomer which functions as a substrate,
thereby identifying a moiety that binds to an asymmetric contact interface of the RTK and inhibits ligand-induced trans autophosphorylation of the RTK.

56. The method of claim 55, wherein the moiety inhibits ligand induced trans autophosphorylation of the RTK; or b) does not cause the loss of intrinsic RTK kinase activity.

57. (canceled)

58. A small molecule that binds to an asymmetric contact interface of a receptor tyrosine kinase (RTK), wherein the small molecule inhibits trans autophosphorylation of the RTK.

59. The small molecule of claim 58, wherein the small molecule binds to a) an amino acid residue selected from the group consisting of R577, D519, C488, F489, S518, T521, E522, D554, G555, P556, Q574, P587, P579, W691, T695, P702, G703 and P705 of FGFR1, or within 1-5 Å of said residue; b) an amino acid residue selected from the group consisting of C491, F492, R577, P582, I590, P705, G706 and P708 of FGFR2; or c) a region selected from the group consisting of the β1-β2 loop of a monomer of the RTK, the β3-αC loop of a monomer of the RTK, the β4-B5 loop of a monomer of the RTK, the αD-αE loop of a monomer of the RTK, the αF helix of a monomer of the RTK and the αF-αG loop of a monomer of the RTK.

60-61. (canceled)
